(19) United States
(12) Patent Application Publication
(10) Pub. No.: US 2023/0095295 A1
(43) Pub. Date: Mar. 30, 2023

(54) PHI29 MUTANTS AND USE THEREOF

(71) Applicant: BioSkryb Genomics, Inc., Durham, NC (US)

(72) Inventors: Charles GAWAD, Durham, NC (US); Jay A.A. WEST, Durham, NC (US); Paul MCEWAN, Durham, NC (US)

(21) Appl. No.: 17/798,468

(22) PCT Filed: Feb. 9, 2021
(86) PCT No.: PCT/US2021/017247

§ 371 Date: Aug. 9, 2022

Related U.S. Application Data
(60) Provisional application No. 62/972,557, filed on Feb. 10, 2020.

(51) Int. Cl. C12Q 1/6883; C12N 15/10

(52) U.S. Cl. CPC C12Q 1/6883; C12N 15/1065; C12Q 1/6858

(57) ABSTRACT

Provided herein are compositions and methods using mutant Phi29 polymerases for nucleic acid amplification. Further provided herein are methods for accurate and scalable Primary Template-Directed Amplification (PTA) nucleic acid amplification and sequencing methods, and their applications for mutational analysis in research, diagnostics, and treatment using mutant Phi29 polymerases.

### CROSS-REFERENCE

This application claims the benefit of U.S. provisional patent application No. 62/972,557 filed on Feb. 10, 2020, which is incorporated by reference in its entirety.

### SEQUENCE LISTING

The instant application contains a Sequence Listing which has been submitted electronically in ASCII format and is hereby incorporated by reference in its entirety. Said ASCII copy, created on Jan. 28, 2021, is named 55461-704_601_SL.txt and is 33,771 bytes in size.

### BACKGROUND

Research methods that utilize nucleic amplification, e.g., Next Generation Sequencing, provide large amounts of information on complex samples, genomes, and other nucleic acid sources. However, there is a need for highly accurate, scalable, and efficient nucleic acid amplification and sequencing methods for research, diagnostics, and treatment involving small samples.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF SUMMARY

Provided herein are methods of nucleic acid amplification comprising: (a) providing a sample comprising at least one target nucleic acid molecule; (b) contacting the sample with at least one amplification primer, at least one polymerase, and a mixture of nucleotides, wherein the mixture of nucleotides comprises at least one terminator nucleotide which terminates nucleic acid replication by the polymerase, wherein the polymerase comprises at least three mutations relative to SEQ ID NO:1, wherein at least two mutations are at positions 370-395 relative to SEQ ID NO: 1, and wherein the polymerase has increased processivity, increased strand displacement activity, increased template or primer binding, decreased error rate, increased 3′->5′ exonuclease activity, increased nucleotide selectivity, or increased temperature stability relative to a polymerase comprising SEQ ID NO: 1 and (c) amplifying the at least one target nucleic acid molecule to generate a plurality of terminated amplification products. Further provided herein are methods wherein increased nucleotide selectivity comprises increased affinity for non-canonical nucleotides. Further provided herein are methods wherein the non-canonical nucleotides comprise dideoxynucleotides. Further provided herein are methods further comprising ligating the molecules obtained in step (c) to adaptors, thereby generating a library of amplification products. Further provided herein are methods wherein the method further comprises sequencing the library of amplification products. Further provided herein are methods wherein the method further comprises comparing the sequences of amplification products to at least one reference sequence to identify at least one mutation. Further provided herein are methods wherein the sample comprises genomic DNA. Further provided herein are methods wherein the sample is a single cell. Further provided herein are methods wherein the single cell is a mammalian cell. Further provided herein are methods wherein the single cell is a human cell. Further provided herein are methods wherein at least some of the amplification products comprise a barcode. Further provided herein are methods wherein at least some of the amplification products comprise at least two barcodes. Further provided herein are methods wherein the barcode comprises a cell barcode. Further provided herein are methods wherein the barcode comprises a sample barcode. Further provided herein are methods wherein at least some of the amplification primers comprise a unique molecular identifier (UMI). Further provided herein are methods wherein at least some of the amplification primers comprise at least two unique molecular identifiers (UMIs). Further provided herein are methods wherein the method further comprises an additional amplification step using PCR. Further provided herein are methods wherein the method further comprises removing at least one terminator nucleotide from the terminated amplification products prior to ligation to adapters. Further provided herein are methods wherein single cells are isolated from the population using a method comprising a microfluidic device. Further provided herein are methods wherein the at least one mutation occurs in no more than 1% of the amplification product sequences. Further provided herein are methods wherein the at least one mutation occurs in no more than 0.1% of the amplification product sequences. Further provided herein are methods wherein the at least one mutation occurs in no more than 0.01% of the amplification product sequences. Further provided herein are methods wherein the at least one mutation occurs in no more than 0.001% of the amplification product sequences. Further provided herein are methods wherein the at least one mutation occurs in no more than 0.0001% of the amplification product sequences. Further provided herein are methods wherein the at least one mutation is present in a region of a sequence correlated with a genetic disease or condition.

Provided herein are variant polymerases comprising SEQ ID NO: 1, wherein the polymerase comprises at least two mutations at positions 370-395 relative to SEQ ID NO: 1, and wherein the polymerase has increased processivity, increased strand displacement activity, increased template or primer binding, decreased error rate, increased 3′->5′ exonuclease activity, increased nucleotide selectivity, or increased temperature stability relative to a polymerase comprising SEQ ID NO: 1. Further provided herein are polymerases wherein the polymerase comprises at least three mutations at positions 370-395 relative to SEQ ID NO: 1. Further provided herein are polymerases wherein the polymerase comprises at least four mutations at positions 370-395 relative to SEQ ID NO: 1. Further provided herein are polymerases wherein at least one mutation is at positions 1-369 or 396-575 relative to SEQ ID NO: 1. Further provided herein are polymerases wherein the at least one mutation comprises a substitution, deletion, or addition. Further provided herein are polymerases wherein the at least one mutation is at positions A382, L386, M385, or E375. Further provided herein are polymerases wherein the at least one mutation comprises at least one substitution. Further provided herein are polymerases wherein the at least one substitution is at an alanine, glycine, leucine, methionine, glutamic acid, or cysteine position of SEQ ID NO: 1. Further provided herein are polymerases wherein the at least one substitution is from alanine, glycine, leucine, methionine, glutamic acid, or cysteine to phenylalanine, tyrosine, or tryptophan. Further provided herein are polymerases wherein the polymerase comprises a mutation at P300. Further provided herein are polymerases wherein the polymerase comprises a substitution at P300. Further provided herein are polymerases wherein the polymerase comprises a substitution at P300 to leucine, isoleucine, alanine, glycine, methionine, or cysteine. Further provided herein are polymerases wherein the polymerase comprises a mutation at K512. Further provided herein are polymerases wherein the polymerase comprises a substitution at K512. Further provided herein are polymerases wherein the polymerase comprises a substitution at K512 to alanine, aspartic acid, glutamic acid, tryptophan, tyrosine, phenylalanine, leucine, or histidine. Further provided herein are polymerases wherein the polymerase comprises at least one mutation at M8, V51, M97, L123, G197, K209, E221, E239, Q497, K512, E515, or F526. Further provided herein are polymerases wherein the at least one mutation at M8, V51, M97, L123, G197, K209, E221, E239, Q497, K512, E515, or F526 is at least one substitution. Further provided herein are polymerases wherein the at least one substitution is M8R, V51A, M97T, L123S, G197D, K209E, E221K, E239G, Q497P, K512E, E515A, or F526L. Further provided herein are polymerases wherein the polymerase comprises at least one mutation at M8, D12, N62, M97, M102, H116, K135, H149, K157, M188, I242, S252, Y254, G320, L328, I370, K371, T372, K373, S374, E375, T368, Y369, T372, T373, I378, K379, N387, Y390, Y405, E408, G413, D423, I442, Y449, D456, K478, L480, V509, D510, K512, V514, E515, M554. Further provided herein are polymerases wherein the at least one mutation is at least one substitution. Further provided herein are polymerases wherein the at least one substitution is D12A/E375W/T372D; D12A/E375W/T372E; D12A/E375W/T372R/K478D; D12A/E375W/T372R/K478E; D12A/E375W/T372K/K478D; D12A/E375W/T372K/D478E; D12A/E375W/K135D; D12A/E375W/K135E; D12A/E375W/K512D; D12A/E375W/K512E; D12A/E375W/E408K; D12A/E375W/E408R; D12A/E375W/T368D/L480K; D12A/E375W/T368E/L480K; D12A/D456N; N62D/D456N; D12A/D456A; N62D/D456A; D12A/D456S; N62D/D456S; N62D/E375M; N62D/E375L; N62D/E375I; N62D/E375F; N62D/E375D; D12A/K512W; N62D/K512W; D12A/K512Y; N62D/K512Y; D12A/K512F; N62D/K512F; D12A/E375W/K512L; N62D/E375W/K512L; D12A/E375W/K512Y; N52D/E375W/K512Y; D12A/E375W/K512F; N62D/E375W/K512F; D12A/E375Y/K512L; N62D/E375Y/K512L; D12A/E375Y/K512Y; N62D/E375Y/K512Y; D12A/E375Y/K512F; N62D/E375Y/K512F; D12A/E375W/K512H; N62D/E375W/K512H; D12A/E375Y/K512H; N62D/E375Y/K512H; D12A/D510F; N62D/D510F; D12A/D510Y; N62D/D510Y; D12A/D510W; N62D/D510W; D12A/E375W/D510F; N62D/E375W/D510F; D12A/E375W/D510Y; N62D/E375W/D510Y; D12A/E375W/D510W; N62D/E375W/D510W; D12A/E375W/D510W/K512L; N62D/E375W/D510W/K512L; D12A/E375W/D510W/K512F; N62D/E375W/D510W/K512F; D12A/E375W/D510H; N62D/E375W/D510H; D12A/E375W/D510H/K512H; N62D/E375W/D510H/K512H; D12A/E375W/D510H/K512F; N62D/E375W/D510H/K512F; D12A/V509Y; N62D/V509Y; D12A/V509W; N62D/V509W; D12A/V509F; N62D/V509F; D12A/V514Y; N62D/V514Y; D12A/V514W; N62D/V514W; D12A/V514F; N62D/V514F; D12S; D12N; D12Q; D12K; D12A/N62D/Y254F; N62D/Y254V; N62D/Y254A; N62D/Y390F; N62D/Y390A; N62D/S252A; N62D/N387A; N62D/K157E; N62D/I242H; N62D/Y259S; N62D/G320C; N62D/L328V; N62D/T368M; N62D/T368G; N62D/Y369R; N62D/Y369H; N62D/Y369E; N62D/I370V; N62D/I370K; N62D/K371Q; N62D/T372N; N62D/T372D; N62D/T372R; N62D/T372L; N62D/T373A; N62D/T373H; N62D/S374E; N62D/I378K; N62D/K379E; N62D/K379T; N62D/N387D; N62D/Y405V; N62D/L408D; N62D/G413D; N62D/D423V; N62D/I442V; N62D/Y449F; N62D/D456V; N62D/L480M; N62D/V509K; N62D/V509I; N62D/D510A; N62D/V514I; N62D/V514K; N62D/E515K; N62D/D523T; N62D/H149Y/E375W/M554S; M8S/N62D/M102S/H116Y/M188S/E375W; N62D/M97S/E375W; M8S/N62D/M97S/M102S/M188S/E375W/M554S; or M8AN62D/M97A/M102A/M188A/E375W/M554A.

Provided herein are variant polymerases, wherein the polymerase comprises a sequence having at least 70% identity to any one of SEQ ID NOS: 4-15. Further provided herein are polymerases wherein the polymerase comprises a sequence having at least 80% identity to any one of SEQ ID NOS: 4-15. Further provided herein are polymerases wherein the polymerase comprises a sequence having at least 90% identity to any one of SEQ ID NOS: 4-15. Further provided herein are polymerases wherein the polymerase comprises a sequence having at least 95% identity to any one of SEQ ID NOS: 4-15. Further provided herein are polymerases wherein the polymerase comprises a sequence having at least 97% identity to any one of SEQ ID NOS: 4-15.

Provided herein are variant polymerases, wherein the polymerase comprises a sequence of any one of SEQ ID NOS: 4-10.

Provided herein are variant polymerases, wherein the polymerase comprises a sequence of any one of SEQ ID NOS: 11-15.

Provided herein are variant polymerases comprising a polypeptide having the structure of Formula I: X¹X²X³X⁴X⁵X⁶X⁷X⁸X⁹X¹⁰X¹¹X¹²X¹³X¹⁴X¹⁵X¹⁶X¹⁷X¹⁸X¹⁹X²⁰X²¹X²²X²³X²⁴X²⁵X²⁶ Formula (I); wherein X¹, X⁷, X⁸, X⁹, X¹², X¹³, X¹⁵, X¹⁶, X¹⁷, X²⁰, X²¹, X²², X²⁴ and X²⁵ are each independently an aromatic or non-polar amino acid; X³, X⁴, X⁵, X¹¹, X¹⁸, X¹⁹, and X²⁶ are each independently polar amino acids; X², X¹⁰, X¹⁴, and X²³ are each independently positively charged amino acids; and X⁶ is an aromatic or negatively charged amino acid, and wherein the polymerase comprises increased processivity, increased strand displacement activity, increased template or primer binding, decreased error rate, increased 3′->5′ exonuclease activity, increased nucleotide selectivity, or increased temperature stability relative to a polymerase comprising SEQ ID NO: 1. Further provided herein are polymerases wherein X²¹ and X²⁴ are each independently a non-polar aromatic amino acid. Further provided herein are polymerases wherein at least one of X¹, X⁷, X⁸, X⁹, X¹², X¹³, X¹⁵, X¹⁶, X¹⁷, X²⁰, X²¹, X²⁵ are each independently an aromatic amino acid. Further provided herein are polymerases wherein at least one of X¹, X⁷, X⁸, X⁹, X¹², X¹³, X¹⁵, X¹⁶, X¹⁷, X²⁰, X²¹, X²⁵ are each independently tyrosine, phenylalanine, or tryptophan. Further provided herein are polymerases wherein at least one of X¹, X⁷, X⁸, X⁹, X¹², and X¹³ are each independently tyrosine, phenylalanine, or tryptophan. Further provided herein are polymerases wherein at least one of X¹⁵, X¹⁶, X¹⁷, X²⁰, X²¹, X²⁵ are each independently tyrosine, phenylalanine, or tryptophan. Further provided herein are polymerases wherein at least two of X¹, X⁷, X⁸, X⁹, X¹², X¹³, X¹⁵, X¹⁶, X¹⁷, X²⁰, X²¹, X²⁵ are each independently tyrosine, phenylalanine, or tryptophan. Further provided herein are polymerases wherein at least one of X¹, X⁶, X⁷, X⁸, X⁹, X¹², X¹³, X¹⁵, X¹⁶, X¹⁷, X²⁰, X²¹, X²⁵ are each independently tyrosine, phenylalanine, or tryptophan. Further provided herein are polymerases wherein at least one of X¹, X⁷, X⁸, X⁹, X¹², X¹³, X¹⁵, X¹⁶, X¹⁷, X²⁰, X²¹, X²⁵ are each independently valine or isoleucine. Further provided herein are polymerases wherein X¹⁶ is an aromatic amino acid. Further provided herein are polymerases wherein X¹⁶ is tyrosine, phenylalanine, or tryptophan. Further provided herein are polymerases wherein X¹⁷ is glycine or alanine. Further provided herein are polymerases wherein X⁶ is an aromatic amino acid. Further provided herein are polymerases wherein X⁶ is tyrosine, phenylalanine, or tryptophan.

Provided herein are kits for nucleic acid sequencing comprising: at least one amplification primer; at least one variant nucleic acid polymerase described herein; a mixture of at least two nucleotides, wherein the mixture of nucleotides comprises at least one terminator nucleotide which terminates nucleic acid replication by the polymerase; and instructions for use of the kit to perform nucleic acid sequencing. Further provided herein are kits wherein the at least one amplification primer is a random primer. Further provided herein are kits wherein the nucleic acid polymerase is a DNA polymerase. Further provided herein are kits wherein the DNA polymerase is a strand displacing DNA polymerase. Further provided herein are kits wherein the least one terminator nucleotide comprises modifications of the r group of the 3′ carbon of the deoxyribose. Further provided herein are kits wherein the at least one terminator nucleotide is selected from the group consisting of 3′ blocked reversible terminator containing nucleotides, 3′ unblocked reversible terminator containing nucleotides, terminators containing 2′ modifications of deoxynucleotides, terminators containing modifications to the nitrogenous base of deoxynucleotides, and combinations thereof. Further provided herein are kits wherein the at least one terminator nucleotide is selected from the group consisting of dideoxynucleotides, inverted dideoxynucleotides, 3′ biotinylated nucleotides, 3′ amino nucleotides, 3′-phosphorylated nucleotides, 3′-O-methyl nucleotides, 3′ carbon spacer nucleotides including 3′ C3 spacer nucleotides, 3′ C18 nucleotides, 3′ Hexanediol spacer nucleotides, acyclonucleotides, and combinations thereof. Further provided herein are kits wherein the at least one terminator nucleotide are selected from the group consisting of nucleotides with modification to the alpha group, C3 spacer nucleotides, locked nucleic acids (LNA), inverted nucleic acids, 2′ fluoro nucleotides, 3′ phosphorylated nucleotides, 2′-O-Methyl modified nucleotides, and trans nucleic acids. Further provided herein are kits wherein the nucleotides with modification to the alpha group are alpha-thio dideoxynucleotides. Further provided herein are kits wherein the amplification primers are 4 to 70 nucleotides in length. Further provided herein are kits wherein the at least one amplification primer is 4 to 20 nucleotides in length. Further provided herein are kits wherein the at least one amplification primer comprises a randomized region. Further provided herein are kits wherein the randomized region is 4 to 20 nucleotides in length. Further provided herein are kits wherein the randomized region is 8 to 15 nucleotides in length. Further provided herein are kits wherein the kit further comprises a library preparation kit. Further provided herein are kits wherein the library preparation kit comprises one or more of: at least one polynucleotide adapter; at least one high-fidelity polymerase; at least one ligase; a reagent for nucleic acid shearing; and at least one primer, wherein the primer is configured to bind to the adapter. Further provided herein are kits wherein the kit further comprises reagents configured for gene editing.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A illustrates a comparison of a prior multiple displacement amplification (MDA) method with one of the embodiments of the Primary Template-Directed Amplification (PTA) method, namely the PTA-Irreversible Terminator method.

FIG. 1B illustrates a comparison of the PTA-Irreversible Terminator method with a different embodiment, namely the PTA-Reversible Terminator method.

FIG. 1C illustrates a comparison of MDA and the PTA-Irreversible Terminator method as they relate to mutation propagation.

FIG. 1D illustrates the method steps performed after amplification, which include removing the terminator, repairing ends, and performing A-tailing prior to adapter ligation. The library of pooled cells can then undergo hybridization-mediated enrichment for all exons or other specific regions of interest prior to sequencing. The cell of origin of each read is identified by the cell barcode (shown as green and blue sequences).

FIG. 2A shows the size distribution of amplicons after undergoing PTA with addition of increasing concentrations of terminators (top gel). The bottom gel shows size distribution of amplicons after undergoing PTA with addition of increasing concentrations of reversible terminator, or addition of increasing concentrations of irreversible terminator.

FIG. 2B (GC) shows comparison of GC content of sequenced bases for MDA and PTA.

FIG. 2C shows map quality scores(e) (mapQ) mapping to human genome (p_mapped) after single cells underwent PTA or MDA.

FIG. 2D percent of reads mapping to human genome (p_mapped) after single cells underwent PTA or MDA.

FIG. 2E (PCR) shows the comparison of percent of reads that are PCR duplicates for 20 million subsampled reads after single cells underwent MDA and PTA.

FIG. 3A shows map quality scores(c) (mapQ2) mapping to human genome (p_mapped2) after single cells underwent PTA with reversible or irreversible terminators.

FIG. 3B shows percent of reads mapping to human genome (p_mapped2) after single cells underwent PTA with reversible or irreversible terminators.

FIG. 3C shows a series of box plots describing aligned reads for the mean percent reads overlapping with Alu elements using various methods. PTA had the highest number of reads aligned to the genome.

FIG. 3D shows a series of box plots describing PCR duplications for the mean percent reads overlapping with Alu elements using the various methods.

FIG. 3E shows a series of box plots describing GC content of reads for the mean percent reads overlapping with Alu elements using various methods.

FIG. 3F shows a series of box plots describing the mapping quality of mean percent reads overlapping with Alu elements using various methods. PTA had the highest mapping quality of methods tested.

FIG. 3G shows a comparison of SC mitochondrial genome coverage breadth with different WGA methods at a fixed 7.5× sequencing depth.

FIG. 4 shows mean coverage depth of 10 kilobase windows across chromosome 1 after selecting for a high-quality MDA cell (representative of ˜50% cells) compared to a random primer PTA-amplified cell after downsampling each cell to 40 million paired reads. The figure shows that MDA has less uniformity with many more windows that have more (box A) or less (box C) than twice the mean coverage depth. There is absence of coverage in both MDA and PTA at the centromere due to high GC content and low mapping quality of repetitive regions (box B).

FIG. 5 (Part A) shows beads with oligonucleotides attached with a cleavable linker, unique cell barcode, and a random primer. Part B shows a single cell and bead encapsulated in the same droplet, followed by lysis of the cell and cleavage of the primer. The droplet may then be fused with another droplet comprising the PTA amplification mix. Part C shows droplets are broken after amplification, and amplicons from all cells are pooled. The protocol according to the disclosure is then utilized for removing the terminator, end repair, and A-tailing prior to adapter ligation. The library of pooled cells then undergoes hybridization-mediated enrichment for exons of interest prior to sequencing. The cell of origin of each read is then identified using the cell barcode.

FIG. 6A demonstrates the incorporation of cellular barcodes and/or unique molecular identifiers into the PTA reactions using primers comprising cellular barcodes and/or or unique molecular identifiers.

FIG. 6B demonstrates the incorporation of cellular barcodes and/or unique molecular identifiers into the PTA reactions using hairpin primers comprising cellular barcodes and/or or unique molecular identifiers.

### DETAILED DESCRIPTION OF THE INVENTION

There is a need to develop new scalable, accurate and efficient methods for nucleic acid amplification (including single-cell and multi-cell genome amplification) and sequencing which would overcome limitations in the current methods by increasing sequence representation, uniformity and accuracy in a reproducible manner. Provided herein are compositions and methods for providing accurate and scalable Primary Template-Directed Amplification (PTA) and sequencing. Such methods and compositions facilitate highly accurate amplification of target (or “template”) nucleic acids, which increases accuracy and sensitivity of downstream applications, such as Next-Generation Sequencing. These amplifications are facilitated by polymerases, such as Phi29 polymerase or variants thereof. Further provided herein are methods of single nucleotide variant determination, copy number variation, structural variation, clonotyping, and measurement of environmental mutagenicity. Measurement of genome variation by PTA may be used for various applications, such as, environmental mutagenicity, predicting safety of gene editing techniques, measuring cancer treatment-mediated genomic changes, measuring carcinogenicity of compounds or radiation including genotoxicity studies for determining the safety of new foods or drugs, estimating ages, analysis of resistant bacteria, and identification of bacteria in the environment for industrial applications. Further, these methods may be used to detect selection of specific cellular populations after changes in environmental conditions, such as exposure to anti-cancer treatment, as well as to predict response to immunotherapy based on the mutation and neoantigen burden in single cancer cells.

Definitions

Unless defined otherwise, all technical and scientific terms used herein have the same meaning as is commonly understood by one of ordinary skill in the art to which these inventions belong.

Throughout this disclosure, numerical features are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the invention, subject to any specifically excluded limit in the stated range. Where the stated range includes one or both of the limits, ranges excluding either or both of those included limits are also included in the invention, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

The terms “subject” or “patient” or “individual”, as used herein, refer to animals, including mammals, such as, e.g., humans, veterinary animals (e.g., cats, dogs, cows, horses, sheep, pigs, etc.) and experimental animal models of diseases (e.g., mice, rats). In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A practical Approach, Volumes I and II (D. N. Glover ed. 1985); Oligonucleotide Synthesis (M J. Gait ed. 1984); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; B. Perbal, A practical Guide To Molecular Cloning (1984); F. M. Ausubel et al. (eds.), Current Protocols in Molecular Biology, John Wiley & Sons, Inc. (1994); among others.

The term “nucleic acid” encompasses multi-stranded, as well as single-stranded molecules. In double- or triple-stranded nucleic acids, the nucleic acid strands need not be coextensive (i.e., a double-stranded nucleic acid need not be double-stranded along the entire length of both strands). Nucleic acid templates described herein may be any size depending on the sample (from small cell-free DNA fragments to entire genomes), including but not limited to 50-300 bases, 100-2000 bases, 100-750 bases, 170-500 bases, 100-5000 bases, 50-10,000 bases, or 50-2000 bases in length. In some instances, templates are at least 50, 100, 200, 500, 1000, 2000, 5000, 10,000, 20,000 50,000, 100,000, 200,000, 500,000, 1,000,000 or more than 1,000,000 bases in length. Methods described herein provide for the amplification of nucleic acid acids, such as nucleic acid templates. Methods described herein additionally provide for the generation of isolated and at least partially purified nucleic acids and libraries of nucleic acids. Nucleic acids include but are not limited to those comprising DNA, RNA, circular RNA, mtDNA (mitochondrial DNA), cfDNA (cell free DNA), cfRNA (cell free RNA), siRNA (small interfering RNA), cffDNA (cell free fetal DNA), mRNA, tRNA, rRNA, miRNA (microRNA), synthetic polynucleotides, polynucleotide analogues, any other nucleic acid consistent with the specification, or any combinations thereof. The length of polynucleotides, when provided, are described as the number of bases and abbreviated, such as nt (nucleotides), bp (bases), kb (kilobases), or Gb (gigabases).

The term “droplet” as used herein refers to a volume of liquid on a droplet actuator. Droplets in some instances, for example, be aqueous or non-aqueous or may be mixtures or emulsions including aqueous and non-aqueous components. For non-limiting examples of droplet fluids that may be subjected to droplet operations, see, e.g., Int. Pat. Appl. Pub. No. WO2007/120241. Any suitable system for forming and manipulating droplets can be used in the embodiments presented herein. For example, in some instances a droplet actuator is used. For non-limiting examples of droplet actuators which can be used, see, e.g., U.S. Pat. Nos. 6,911,132, 6,977,033, 6,773,566, 6,565,727, 7,163,612, 7,052,244, 7,328,979, 7,547,380, 7,641,779, U.S. Pat. Appl. Pub. Nos. US20060194331, US20030205632, US20060164490, US20070023292, US20060039823, US20080124252, US20090283407, US20090192044, US20050179746, US20090321262, US20100096266, US20110048951, Int. Pat. Appl. Pub. No. WO2007/120241. In some instances, beads are provided in a droplet, in a droplet operations gap, or on a droplet operations surface. In some instances, beads are provided in a reservoir that is external to a droplet operations gap or situated apart from a droplet operations surface, and the reservoir may be associated with a flow path that permits a droplet including the beads to be brought into a droplet operations gap or into contact with a droplet operations surface. Non-limiting examples of droplet actuator techniques for immobilizing magnetically responsive beads and/or non-magnetically responsive beads and/or conducting droplet operations protocols using beads are described in U.S. Pat. Appl. Pub. No. US20080053205, Int. Pat. Appl. Pub. No. WO2008/098236, WO2008/134153, WO2008/116221, WO2007/120241. Bead characteristics may be employed in the multiplexing embodiments of the methods described herein. Examples of beads having characteristics suitable for multiplexing, as well as methods of detecting and analyzing signals emitted from such beads, may be found in U.S. Pat. Appl. Pub. No. US20080305481, US20080151240, US20070207513, US20070064990, US20060159962, US20050277197, US20050118574.

As used herein, the term “unique molecular identifier (UMI)” refers to a unique nucleic acid sequence that is attached to each of a plurality of nucleic acid molecules. When incorporated into a nucleic acid molecule, an UMI in some instances is used to correct for subsequent amplification bias by directly counting UMIs that are sequenced after amplification. The design, incorporation and application of UMIs is described, for example, in Int. Pat. Appl. Pub. No. WO 2012/142213, Islam et al. Nat. Methods (2014) 11:163-166, and Kivioja, T. et al. Nat. Methods (2012) 9: 72-74.

As used herein, the term “barcode” refers to a nucleic acid tag that can be used to identify a sample or source of the nucleic acid material. Thus, where nucleic acid samples are derived from multiple sources, the nucleic acids in each nucleic acid sample are in some instances tagged with different nucleic acid tags such that the source of the sample can be identified. Barcodes, also commonly referred to indexes, tags, and the like, are well known to those of skill in the art. Any suitable barcode or set of barcodes can be used. See, e.g., non-limiting examples provided in U.S. Pat. No. 8,053,192 and Int. Pat. Appl. Pub. No. WO2005/068656. Barcoding of single cells can be performed as described, for example, in U.S. Pat. Appl. Pub. No. 2013/0274117.

The terms “solid surface,” “solid support” and other grammatical equivalents herein refer to any material that is appropriate for or can be modified to be appropriate for the attachment of the primers, barcodes and sequences described herein. Exemplary substrates include, but are not limited to, glass and modified or functionalized glass, plastics (including acrylics, polystyrene and copolymers of styrene and other materials, polypropylene, polyethylene, polybutylene, polyurethanes, Teflon™, etc.), polysaccharides, nylon, nitrocellulose, ceramics, resins, silica, silica-based materials (e.g., silicon or modified silicon), carbon, metals, inorganic glasses, plastics, optical fiber bundles, and a variety of other polymers. In some embodiments, the solid support comprises a patterned surface suitable for immobilization of primers, barcodes and sequences in an ordered pattern.

As used herein, the term “biological sample” includes, but is not limited to, tissues, cells, biological fluids and isolates thereof. Cells or other samples used in the methods described herein are in some instances isolated from human patients, animals, plants, soil or other samples comprising microbes such as bacteria, fungi, protozoa, etc. In some instances, the biological sample is of human origin. In some instances, the biological is of non-human origin. The cells in some instances undergo PTA methods described herein and sequencing. Variants detected throughout the genome or at specific locations can be compared with all other cells isolated from that subject to trace the history of a cell lineage for research or diagnostic purposes.

The term “identity” or “homology” refer to the percentage of amino acid residues in the candidate sequence that are identical with the residue of a corresponding sequence to which it is compared, after aligning the sequences and introducing gaps, if necessary to achieve the maximum percent identity for the entire sequence, and not considering any conservative substitutions as part of the sequence identity. Conservative substitutions in some instances involve substitution of one amino acid of similar shape (e.g., tyrosine for phenylalanine) or charge (glutamic acid for aspartic acid) for another. A polynucleotide or polynucleotide region (or a peptide or peptide region) comprises a certain percentage (for example, 80%, 85%, 90%, or 95%) of “sequence identity” or “homology” to another sequence means that, when aligned, that percentage of bases (or amino acids) are the same in comparing the two sequences. Neither N- or C-terminal extensions nor insertions shall be construed as reducing identity or homology. Alignment and the percent homology or sequence identity in some instances are determined using software programs known by those skilled the art. In some instances, default parameters are used for alignment. An exemplary alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Similarity, or percent similarity in some instances of two sequences is the sum of both identical and similar matches (residues that have undergone conservative substitution). In some instances, similarity is measured using the program BLAST “Positives.”

Polypeptides described herein (e.g., Phi29 polymerase variants) comprise amino acids. Such polypeptides may differ from another peptide by one or more amino acid or nucleic acid deletions, additions, substitutions or side-chain modifications, yet retains one or more specific functions or biological activities of the molecule. Amino acid substitutions include alterations in which an amino acid is replaced with a different amino acid residue. Such substitutions in some instances are classified as conservative, in which case an amino acid residue contained in a peptide or peptide is replaced with another naturally occurring amino acid of similar character either in relation to polarity, side chain functionality or size. Such conservative substitutions are well known in the art. Substitutions encompassed by the present disclosure may also be non-conservative, in which an amino acid residue which is present in a peptide is substituted with an amino acid having different properties, such as an amino acid from a different group (e.g., substituting a charged or hydrophobic amino; acid with alanine). In some instances, amino acid substitutions are conservative. Also encompassed within the term variant when used with reference to a polynucleotide or peptide, refers to a polynucleotide or peptide that can vary in primary, secondary, or tertiary structure, as compared to a reference polynucleotide or peptide, respectively (e.g., as compared to a wild-type polynucleotide or peptide).

Phi29 polymerase variants described herein may comprise insertions, deletions, or substitutions. In some instances, insertions and deletions are in the range of about 1 to 5 amino acids. The variation allowed in some instances is experimentally determined by producing the peptide synthetically while systematically making insertions, deletions, or substitutions of nucleotides in the sequence using recombinant DNA techniques. In some instances, substitution comprises a change in an amino acid for a different entity, for example another amino acid or amino-acid moiety. Substitutions can be conservative or non-conservative substitutions. In some instances, the peptide is a variant comprising at least one amino acid substitution, deletion, or insertion relative to the amino acid sequence of any one of SEQ ID NOS: 1-15. Variants can include conservative or non-conservative amino acid changes, as described below. In some instances, a variant does not comprise a naturally-occurring protein sequence, such as Phi29 polymerase (SEQ ID NO: 1). Polynucleotide changes can result in amino acid substitutions, additions, deletions, fusions and truncations in the peptide encoded by the reference sequence. The term conservative substitution, when describing a peptide, refers to a change in the amino acid composition of the peptide that does not substantially alter the peptide's activity. For example, a conservative substitution refers to substituting an amino acid residue for a different amino acid residue that has similar chemical properties. Conservative amino acid substitutions include replacement of a leucine with an isoleucine or valine, an aspartate with a glutamate, or a threonine with a serine. Conservative amino acid substitutions result from replacing one amino acid with another having similar structural and/or chemical properties, such as the replacement of a leucine with an isoleucine or valine, an aspartate with a glutamate, or a threonine with a serine. Thus, a conservative substitution of a particular amino acid sequence refers to substitution of those amino acids that are not critical for peptide activity or substitution of amino acids with other amino acids having similar properties (e.g., acidic, basic, positively or negatively charged, polar or non-polar) such that the substitution of even critical amino acids does not reduce the activity of the peptide. Conservative substitution tables providing functionally similar amino acids are well known in the art. For example, the following six groups each contain amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W). Groups of amino acids are categorized in some instances based on polarity or charge of their respective side chains. In some instances, non-polar amino acids include but are not limited to Glycine, Alanine, Valine, Leucine, Isoleucine, Methionine, Phenylalanine, Tryptophan, or Proline. In some instances polar amino acids include but are not limited to Serine, Threonine, Cysteine, Tryptophan, Asparagine, or Glutamine. In some instances positively charged amino acids include but are not limited to Lysine, Arginine, or Histidine. In some instances negatively charged amino acid include but are not limited to Aspartic acid or Glutamic acid. In some instances, an amino acid is a negatively charged amino acid. In some instances, negatively charged amino acids comprise side-chain functional groups which are negatively charged under aqueous physiological conditions (e.g., pH˜7), such as carboxylic acids.

In some instances, an amino acid is a positively charged amino acid. In some instances, positively charged amino acids comprise side-chain functional groups which are positively charged under aqueous physiological conditions (e.g., pH˜7). In some instances, positively charged amino acids comprise basic functional group side chains. In some instances, basic functional groups include but are not limited to amines (substituted or unsubstituted), pyrrolidines, or other basic functional group.

In some instances, individual substitutions, deletions or additions that alter, add or delete a single amino acid or a small percentage of amino acids can also be considered conservative substitutions if the change does not significantly reduce the activity of the peptide. Insertions or deletions are typically in the range of about 1 to 5 amino acids. The choice of conservative amino acids in some instances is selected based on the location of the amino acid to be substituted in the peptide, for example if the amino acid is on the exterior of the peptide and expose to solvents, or on the interior and not exposed to solvents. In some instances, one can select the amino acid which will substitute an existing amino acid based on the location of the existing amino acid, i.e. its exposure to solvents (i.e. if the amino acid is exposed to solvents or is present on the outer surface of the peptide or peptide as compared to internally localized amino acids not exposed to solvents). Selection of such conservative amino acid substitutions are well known in the art. Accordingly, one can select conservative amino acid substitutions suitable for amino acids on the exterior of a protein or peptide (i.e. amino acids exposed to a solvent). For example, but not limited to, the following substitutions can be used: substitution of Y with F, T with S or K, P with A, E with D or Q, N with D or G, R with K, G with N or A, T with S or K, D with N or E, I with L or V, F with Y, S with Tor A, R with K, G with N or A, K with R, A with S, K or P. In some instances a conservative amino acid substitution is suitable for amino acids on the interior of a protein or peptide, for example suitable conservative substitutions for amino acids in some instances are on the interior of a protein or peptide (i.e. the amino acids are not exposed to a solvent). For example but not limited to, one can use the following conservative substitutions: where Y is substituted with F, T with A or S, I with L or V, W with Y, M with L, N with D, G with A, T with A or S, D with N, I with L or V, F with Y or L, S with A or T and A with S, G, Tor V. In some instances, nonconservative amino acid substitutions are also encompassed within the term of variants.

In some aspects, the peptides or peptides disclosed herein are derivatives of the SEQ ID NOS:1-15. The term derivative in some instances comprises peptides which have been chemically modified, for example but not limited to by techniques such as ubiquitination, labeling, pegylation (i.e., derivatization with polyethylene glycol), lipidation, glycosylation, or addition of other molecules. A molecule is also in some instances a derivative of another molecule when it contains additional chemical moieties not normally a part of the molecule. Such moieties can improve the molecule's potency, solubility, absorption, biological half-life, etc. In some instances, a peptide described herein comprises a half-life extending moiety (e.g., water soluble polymer, lipid, protein, or peptide). The moieties can alternatively decrease the toxicity of the molecule, eliminate or attenuate any undesirable side effect of the molecule, increase antibiotic spectrum, or have other effects.

Amino acid substitutions may be made in a polypeptide (e.g., Phi29 polymerase) at one or more positions wherein the substitution is for an amino acid having a similar hydrophilicity. The importance of the hydropathic amino acid index in conferring interactive biologic function on a protein is generally understood in the art. In some instances, the relative hydropathic character of the amino acid contributes to the secondary structure of the resultant protein, which in turn defines the interaction of the protein with other molecules, for example, enzymes, substrates, receptors, DNA, antibodies, antigens, and the like. Thus such conservative substitution can be made in a polypeptide and will likely only have minor effects on their activity. For example, the following hydrophilicity values may be assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (0.5); histidine −0.5); cysteine (−1.0); methionine (−1.3); valine (−1.5); leucine (−1.8); isoleucine (−1.8); tyrosine (−2.3); phenylalanine (−2.5); tryptophan (−3.4). These values can be used as a guide and thus substitution of amino acids whose hydrophilicity values are within ±2 are preferred, those that are within ±1 are particularly preferred, and those within ±0.5 are even more particularly preferred. Thus, any of the peptides or peptides described herein in some instances are modified by the substitution of an amino acid, for a different, but homologous amino acid with a similar hydrophilicity value. Amino acids with hydrophilicities within +/−1.0, or +/−0.5 points are considered homologous. The Phi29 polymerase variants described herein may comprise additional modifications. In some instances, a modification comprises a co-translational and/or post-translational (C-terminal peptide cleavage) modification. In some instances, a modification includes but is not limited to a disulfide bond formation, backbone cyclization, glycosylation, acetylation, phosphorylation, and proteolytic cleavage (e.g., cleavage by furins or metalloproteases).

Mutant Phi29 Polymerases

Described herein are polymerases for amplification of polynucleotide templates. Further described herein are variant Phi29 polymerases. In some instances, polymerases described herein comprise one or more mutations from a wild-type sequence. In some instances, such mutations result in higher fidelity, rate of amplification, increased processivity, improved strand displacement, stronger template or primer binding, increased 3′->5′ exonuclease activity, altered affinity for specific nucleotides, and greater temperature stability. In some instances, polymerases described herein have increased affinity for unnatural nucleotides. In some instances, polymerases described herein have increased affinity for dideoxynucleotides. In some instances, polymerases described herein comprise a 3′-5′ exonuclease strand displacement domain. In some instances, polymerases described herein comprise a protein-primed initiation and DNA polymerization domain. In some instances, polymerases described herein comprise TPR1 and TPR2 domains. In some instances, polymerases described herein comprise a palm, thumb, and finger structural domains. In some instances, a polymerase described herein comprises a mutation found in the conserved region 370-395 (SEQ ID NO: 2). In some instances, a polymerase comprises a mutation at a residue in SEQ ID NO:2 of a Phi29 polymerase which analogous to a residue found in the conserved region of a Pfu polymerase 471-500 (SEQ ID NO: 3). In some instances, polymerases described herein (e.g., Phi29) control the kinetics of amplification from a sample template. In some instances, polymerases described herein (e.g., Phi29) control the length of amplicons from a sample template.

Described herein are variants of polymerase Phi29, wherein one or more residues in the peptide chain are added, deleted, or substituted with a different amino acid. In some instances, a polymerase variant described herein comprises a polypeptide having the structure of Formula I:

X¹X²X³X⁴X⁵X⁶X⁷X⁸X⁹X¹⁰X¹¹X¹²X¹³X¹⁴X¹⁵X¹⁶X¹⁷X¹⁸X¹⁹X²⁰X²¹X²²X²³X²⁴X²⁵X²⁶ Formula (I);

wherein X¹-X²⁶ are independently any amino acid. In some instances, a polymerase variant described herein comprises SEQ ID NO: 1, wherein residues 370-395 are replaced with the structure of a polypeptide of Formula I. In some instances, a polymerase variant described herein comprises a polypeptide having the structure of Formula I, wherein the variant has at least 99% sequence identity to SEQ ID NO: 1. In some instances, a polymerase variant described herein comprises a polypeptide having the structure of Formula I, wherein the variant has at least 98% sequence identity to SEQ ID NO: 1. In some instances, a polymerase variant described herein comprises a polypeptide having the structure of Formula I, wherein the variant has at least 97% sequence identity to SEQ ID NO: 1. In some instances, a polymerase variant described herein comprises a polypeptide having the structure of Formula I, wherein the variant has at least 95% sequence identity to SEQ ID NO: 1. In some instances, a polymerase variant described herein comprises a polypeptide having the structure of Formula I, wherein the variant has at least 90% sequence identity to SEQ ID NO: 1.

In some instances, a polymerase variant described herein comprises a polypeptide having the structure of Formula I:

X¹X²X³X⁴X⁵X⁶X⁷X⁸X⁹X¹⁰X¹¹X¹²X¹³X¹⁴X¹⁵X¹⁶X¹⁷X¹⁸X¹⁹X²⁰X²¹X²²X²³X²⁴X²⁵X²⁶ Formula (I);

wherein
X¹, X⁷, X⁸, X⁹, X¹², X¹³, X¹⁵, X¹⁶, X¹⁷, X²⁰, X²¹, X²², X²⁴, and X²⁵ are each independently an aromatic or non-polar amino acid;
X³, X⁴, X⁵, X¹¹, X¹⁸, X¹⁹, and X²⁶ are each independently polar amino acids;
X², X¹⁰, X¹⁴, and X²³ are each independently positively charged amino acids; and
X⁶ is an aromatic or negatively charged amino acid.

In some instances of a polypeptide of Formula I, X²¹ and X²⁴ are each independently a non-polar aromatic amino acid. In some instances of a polypeptide of Formula I, at least one of X¹, X⁷, X⁸, X⁹, X¹², X¹³, X¹⁵, X¹⁶, X¹⁷, X²⁰, X²¹, X²⁵ are each independently an aromatic amino acid. In some instances of a polypeptide of Formula I, at least one of X¹, X⁷, X⁸, X⁹, X¹², X¹³, X¹⁵, X¹⁶, X¹⁷, X²⁰, X²¹, X²⁵ are each independently tyrosine, phenylalanine, or tryptophan. In some instances of a polypeptide of Formula I, at least one of X¹, X⁷, X⁸, X⁹, X¹², and X¹³ are each independently tyrosine, phenylalanine, or tryptophan. In some instances of a polypeptide of Formula I, at least one of X¹⁵, ^{X16,} X¹⁷, X²⁰, X²¹, X²⁵ are each independently tyrosine, phenylalanine, or tryptophan.

In some instances of a polypeptide of Formula I, at least two of X¹, X⁷, X⁸, X⁹, X¹², X¹³, X¹⁵, X¹⁶, X¹⁷, X²⁰, X²¹, X²⁵ are each independently tyrosine, phenylalanine, or tryptophan. In some instances of a polypeptide of Formula I, at least one of X¹, X⁶, X⁷, X⁸, X⁹, X¹², X¹³, X¹⁵, X¹⁶, X¹⁷, X²⁰, X²¹, X²⁵ are each independently tyrosine, phenylalanine, or tryptophan. In some instances of a polypeptide of Formula I, at least one of X¹, X⁷, X⁸, X⁹, X¹², X¹³, X¹⁵, X¹⁶, X¹⁷, X²⁰, X²¹, X²⁵ are each independently valine or isoleucine. In some instances of a polypeptide of Formula I, X¹⁶ is tyrosine, phenylalanine, or tryptophan. In some instances of a polypeptide of Formula I, X¹⁷ is glycine or alanine. In some instances of a polypeptide of Formula I, X⁶ is an aromatic amino acid. In some instances of a polypeptide of Formula I, X⁶ is tyrosine, phenylalanine, or tryptophan. In some instances, X¹ is isoleucine, valine, alanine, glycine, cysteine, methionine, or leucine. In some instances, X⁷ is isoleucine, valine, alanine, glycine, cysteine, methionine, or leucine. In some instances, X⁸ is isoleucine, valine, alanine, glycine, cysteine, methionine, or leucine. In some instances, X⁹ is isoleucine, valine, alanine, glycine, cysteine, methionine, or leucine. In some instances, X¹² is isoleucine, valine, alanine, glycine, cysteine, methionine, or leucine. In some instances, X¹³ is isoleucine, valine, alanine, glycine, cysteine, methionine, or leucine. In some instances, X¹⁵ is isoleucine, valine, alanine, glycine, cysteine, methionine, or leucine. In some instances, X¹⁶ is isoleucine, valine, alanine, glycine, cysteine, methionine, or leucine. In some instances, X¹⁷ is isoleucine, valine, alanine, glycine, cysteine, methionine, or leucine. In some instances, X²⁰ is isoleucine, valine, alanine, glycine, cysteine, methionine, or leucine. In some instances, X²¹ is isoleucine, valine, alanine, glycine, cysteine, methionine, or leucine. In some instances, X²⁵ is isoleucine, valine, alanine, glycine, cysteine, methionine, or leucine. In some instances, X² is lysine, histidine, or arginine. In some instances, X¹⁰ is lysine, histidine, or arginine. In some instances, X¹⁴ is lysine, histidine, or arginine. In some instances, X²³ is lysine, histidine, or arginine. In some instances, X³ is threonine, serine, glutamine, or asparagine. In some instances, X⁴ is threonine, serine, glutamine, or asparagine. In some instances, X⁵ is threonine, serine, glutamine, or asparagine. In some instances, X¹¹ is threonine, serine, glutamine, or asparagine. In some instances, X¹⁸ is threonine, serine, glutamine, or asparagine. In some instances, X¹⁹ is threonine, serine, glutamine, or asparagine. In some instances, X²⁶ is threonine, serine, glutamine, or asparagine.

In some instances, a polymerase variant described herein comprises SEQ ID NO: 1, wherein residues 370-395 (SEQ ID NO: 3) are replaced with the structure of a polypeptide of Formula I. In some instances, a polymerase variant described herein comprises SEQ ID NO: 1, wherein residues 370-395 are replaced with the structure of a polypeptide of Formula I, and comprise at least one additional mutation. In some instances, a polymerase variant described herein comprises SEQ ID NO: 1, wherein residues 370-395 are replaced with the structure of a polypeptide of Formula I, and comprise at least one additional substitution. In some instances, a polymerase variant described herein comprises SEQ ID NO: 1, wherein residues 370-395 are replaced with the structure of a polypeptide of Formula I, and comprise at least one additional deletion. In some instances, a polymerase variant described herein comprises SEQ ID NO: 1, wherein residues 370-395 are replaced with the structure of a polypeptide of Formula I, and comprise at least one additional addition. In some instances, a polymerase variant described herein comprises SEQ ID NO: 1, wherein residues 370-395 are replaced with the structure of a polypeptide of Formula I, and a mutation at P300. In some instances, a polymerase variant described herein comprises SEQ ID NO: 1, wherein residues 370-395 are replaced with the structure of a polypeptide of Formula I, and a mutation at P300, wherein the mutation is leucine, methionine. isoleucine, or alanine.

Described herein are variants of polymerase Phi29, wherein one or more residues in the peptide chain are added, deleted, or substituted with a different amino acid. In some instances, a variant described herein is shown in Table 1.

TABLE 1

| SEQ | | |
|---|---|---|
| ID | | |
| NO | Name | Sequence |
| 1 | Native_Phi29 | MKHMPRKMYSCDFETTTKVEDCRVWAY |
| | | GYMNIEDHSEYKIGNSLDEFMAWVLKV |
| | | QADLYFHNLKFDGAFIINWLERNGFKW |
| | | SADGLPNTYNTIISRMGQWYMIDICLG |
| | | YKGKRKIHTVIYDSLKKLPFPVKKIAK |
| | | DFKLTVLKGDIDYHKERPVGYKITPEE |
| | | YAYIKNDIQIIAEALLIQFKQGLDRMT |
| | | AGSDSLKGFKDIITTKKFKKVFPTLSL |
| | | GLDKEVRYAYRGGFTWLNDRFKEKEIG |
| | | EGMVFDVNSLYPAQMYSRLLPYGEPIV |
| | | FEGKYVWDEDYPLHIQHIRCEFELKEG |
| | | YIPTIQIKRSRFYKGNEYLKSSGGEIA |
| | | DLWLSNVDLELMKEHYDLYNVEYISGL |
| | | KFKATTGLFKDFIDKWTYIKTTSEGAI |
| | | KQLAKLMLNSLYGKFASNPDVTGKVPY |
| | | LKENGALGFRLGEEETKDPVYTPMGVF |
| | | ITAWARYTTITAAQACYDRIIYCDTDS |
| | | IHLTGTEIPDVIKDIVDPKKLGYWAHE |
| | | STFKRAKYLRQKTYIQDIYMKEVDGKL |
| | | VEGSPDDYTDIKFSVKCAGMTDKIKKE |
| | | VTFENFKVGFSRKMKPKPVQVPGGVVL |
| | | VDDTFTIK |
| 2 | Pfu_471-500 | TQDPIEKILLDYRQKAIKLLANSFYGY |
| | | YGYAK |
| 3 | Phi29_370-395 | IKTTSEGAIKQLAKLMLNSLYGKFAS |
| 4 | Phi29_370- | IKTTSEGAIKQLYKLMLNSLYGKFAS |
| | 395_A382Y | |
| 5 | Phi29_370- | IKTTSEGAIKQLWKLMLNSLYGKFAS |
| | 395_A382W | |
| 6 | Phi29_370- | IKTTSEGAIKQLAKLMYNSLYGKFAS |
| | 395_L386Y | |
| 7 | Phi29_370- | IKTTSEGAIKQLAKLMWNSLYGKFAS |
| | 395_L386W | |
| 8 | Phi29_370- | IKTTSEGAIKQLAKLWANSLYGKFAS |
| | 395_M385W/ | |
| | L386A | |
| 9 | Phi29_370- | IKTTSEGAIKQLAKLMLYSLYGKFAS |
| | 395_N387Y | |
| 10 | Phi29_370- | IKTTSEGAIKQLAKLMLWSLYGKFAS |
| | 395_N387W | |
| 11 | Phi29_M385W | MKHMPRKMYSCDFETTTKVEDCRVWAY |
| | | GYMNIEDHSEYKIGNSLDEFMAWVLKV |
| | | QADLYFHNLKFDGAFIINWLERNGFKW |
| | | SADGLPNTYNTIISRMGQWYMIDICLG |
| | | YKGKRKIHTVIYDSLKKLPFPVKKIAK |
| | | DFKLTVLKGDIDYHKERPVGYKITPEE |
| | | YAYIKNDIQIIAEALLIQFKQGLDRMT |
| | | AGSDSLKGFKDIITTKKFKKVFPTLSL |
| | | GLDKEVRYAYRGGFTWLNDRFKEKEIG |
| | | EGMVFDVNSLYPAQMYSRLLPYGEPIV |
| | | FEGKYVWDEDYPLHIQHIRCEFELKEG |
| | | YIPTIQIKRSRFYKGNEYLKSSGGEIA |
| | | DLWLSNVDLELMKEHYDLYNVEYISGL |
| | | KFKATTGLFKDFIDKWTYIKTTSEGAI |
| | | KQLAKLWLNSLYGKFASNPDVTGKVPY |
| | | LKENGALGFRLGEEETKDPVYTPMGVF |
| | | ITAWARYTTITAAQACYDRIIYCDTDS |
| | | IHLTGTEIPDVIKDIVDPKKLGYWAHE |
| | | STFKRAKYLRQKTYIQDIYMKEVDGKL |
| | | VEGSPDDYTDIKFSVKCAGMTDKIKKE |
| | | VTFENFKVGFSRKMKPKPVQVPGGVVL |
| | | VDDTFTIK |
| 12 | Phi29_L386A | MKHMPRKMYSCDFETTTKVEDCRVWAY |
| | | GYMNIEDHSEYKIGNSLDEFMAWVLKV |
| | | QADLYFHNLKFDGAFIINWLERNGFKW |
| | | SADGLPNTYNTIISRMGQWYMIDICLG |
| | | YKGKRKIHTVIYDSLKKLPFPVKKIAK |
| | | DFKLTVLKGDIDYHKERPVGYKITPEE |
| | | YAYIKNDIQIIAEALLIQFKQGLDRMT |
| | | AGSDSLKGFKDIITTKKFKKVFPTLSL |
| | | GLDKEVRYAYRGGFTWLNDRFKEKEIG |
| | | EGMVFDVNSLYPAQMYSRLLPYGEPIV |
| | | FEGKYVWDEDYPLHIQHIRCEFELKEG |
| | | YIPTIQIKRSRFYKGNEYLKSSGGEIA |
| | | DLWLSNVDLELMKEHYDLYNVEYISGL |
| | | KFKATTGLFKDFIDKWTYIKTTSEGAI |
| | | KQLAKLMANSLYGKFASNPDVTGKVPY |
| | | LKENGALGFRLGEEETKDPVYTPMGVF |
| | | ITAWARYTTITAAQACYDRIIYCDTDS |
| | | IHLTGTEIPDVIKDIVDPKKLGYWAHE |
| | | STFKRAKYLRQKTYIQDIYMKEVDGKL |
| | | VEGSPDDYTDIKFSVKCAGMTDKIKKE |
| | | VTFENFKVGFSRKMKPKPVQVPGGVVL |
| | | VDDTFTIK |
| 13 | Phi29_P300L | MKHMPRKMYSCDFETTTKVEDCRVWAY |
| | | GYMNIEDHSEYKIGNSLDEFMAWVLKV |
| | | QADLYFHNLKFDGAFIINWLERNGFKW |
| | | SADGLPNTYNTIISRMGQWYMIDICLG |
| | | YKGKRKIHTVIYDSLKKLPFPVKKIAK |
| | | DFKLTVLKGDIDYHKERPVGYKITPEE |
| | | YAYIKNDIQIIAEALLIQFKQGLDRMT |
| | | AGSDSLKGFKDIITTKKFKKVFPTLSL |
| | | GLDKEVRYAYRGGFTWLNDRFKEKEIG |
| | | EGMVFDVNSLYPAQMYSRLLPYGEPIV |
| | | FEGKYVWDEDYPLHIQHIRCEFELKEG |
| | | YIPTIQIKRSRFYKGNEYLKSSGGEIA |
| | | DLWLSNVDLELMKEHYDLYNVEYISGL |
| | | KFKATTGLFKDFIDKWTYIKTTSEGAI |
| | | KQLAKLWLNSLYGKFASNPDVTGKVPY |
| | | LKENGALGFRLGEEETKDPVYTPMGVF |
| | | ITAWARYTTITAAQACYDRIIYCDTDS |
| | | IHLTGTEIPDVIKDIVDPKKLGYWAHE |
| | | STFKRAKYLRQKTYIQDIYMKEVDGKL |
| | | VEGSPDDYTDIKFSVKCAGMTDKIKKE |
| | | VTFENFKVGFSRKMKPKPVQVPGGVVL |
| | | VDDTFTIK |
| 14 | Phi29_E375W | MKHMPRKMYSCDFETTTKVEDCRVWAY |
| | | GYMNIEDHSEYKIGNSLDEFMAWVLKV |
| | | QADLYFHNLKFDGAFIINWLERNGFKW |
| | | SADGLPNTYNTIISRMGQWYMIDICLG |
| | | YKGKRKIHTVIYDSLKKLPFPVKKIAK |
| | | DFKLTVLKGDIDYHKERPVGYKITPEE |
| | | YAYIKNDIQIIAEALLIQFKQGLDRMT |
| | | AGSDSLKGFKDIITTKKFKKVFPTLSL |
| | | GLDKEVRYAYRGGFTWLNDRFKEKEIG |
| | | EGMVFDVNSLYPAQMYSRLLPYGEPIV |
| | | FEGKYVWDEDYPLHIQHIRCEFELKEG |
| | | YIPTIQIKRSRFYKGNEYLKSSGGEIA |
| | | DLWLSNVDLELMKEHYDLYNVEYISGL |
| | | KFKATTGLFKDFIDKWTYIKTTSWGAI |
| | | KQLAKLMLNSLYGKFASNPDVTGKVPY |
| | | LKENGALGFRLGEEETKDPVYTPMGVF |
| | | ITAWARYTTITAAQACYDRIIYCDTDS |
| | | IHLTGTEIPDVIKDIVDPKKLGYWAHE |
| | | STFKRAKYLRQKTYIQDIYMKEVDGKL |
| | | VEGSPDDYTDIKFSVKCAGMTDKIKKE |
| | | VTFENFKVGFSRKMKPKPVQVPGGVVL |
| | | VDDTFTIK |
| 15 | Phi29_P300L/ | MKHMPRKMYSCDFETTTKVEDCRVWAY |
| | L386A/E375W | GYMNIEDHSEYKIGNSLDEFMAWVLKV |
| | | QADLYFHNLKFDGAFIINWLERNGFKW |
| | | SADGLPNTYNTIISRMGQWYMIDICLG |
| | | YKGKRKIHTVIYDSLKKLPFPVKKIAK |
| | | DFKLTVLKGDIDYHKERPVGYKITPEE |
| | | YAYIKNDIQIIAEALLIQFKQGLDRMT |
| | | AGSDSLKGFKDIITTKKFKKVFPTLSL |
| | | GLDKEVRYAYRGGFTWLNDRFKEKEIG |
| | | EGMVFDVNSLYPAQMYSRLLPYGEPIV |
| | | FEGKYVWDEDYPLHIQHIRCEFELKEG |
| | | YILTIQIKRSRFYKGNEYLKSSGGEIA |
| | | DLWLSNVDLELMKEHYDLYNVEYISGL |
| | | KFKATTGLFKDFIDKWTYIKTTSWGAI |
| | | KQLAKLMANSLYGKFASNPDVTGKVPY |
| | | LKENGALGFRLGEEETKDPVYTPMGVF |
| | | ITAWARYTTITAAQACYDRIIYCDTDS |
| | | IHLTGTEIPDVIKDIVDPKKLGYWAHE |
| | | STFKRAKYLRQKTYIQDIYMKEVDGKL |
| | | VEGSPDDYTDIKFSVKCAGMTDKIKKE |
| | | VTFENFKVGFSRKMKPKPVQVPGGVVL |
| | | VDDTFTIK |

In some instances, a polymerase (e.g., Phi29) comprises a sequence of Table 1. In some instances, a polymerase comprises any one of SEQ ID NOs: 4-10. In some instances, a polymerase comprises any one of SEQ ID NOs: 4-10, and at least one mutation. In some instances, a polymerase comprises any one of SEQ ID NOs: 4-10, and at least one substitution. In some instances, a polymerase comprises any one of SEQ ID NOs: 4-10, and at least one addition. In some instances, a polymerase comprises any one of SEQ ID NOs: 4-10, and at least one deletion. In some instances, a polymerase comprises any one of SEQ ID NOs: 4-10 and a substitution at P300. In some instances, a polymerase comprises any one of SEQ ID NOs: 4-10 and substitution P300L. In some instances, a polymerase comprises any one of SEQ ID NOs: 4-10 and a substitution at K512. In some instances, a polymerase comprises any one of SEQ ID NOs: 4-10 and substitution K512A, K512D, K512E, K512W, K512Y, K512F, K512L, or K512H. In some instances, a polymerase comprises any one of SEQ ID NOs: 4-10 and substitution M8R, V51A, M97T, L123S, G197D, K209E, E221K, E239G, Q497P, K512E, E515A, or F526L. In some instances, a polymerase comprises any one of SEQ ID NOs: 4-10 and a mutation or combination of mutations selected from any one of: D12A/E375W/T372D; D12A/E375W/T372E; D12A/E375W/T372R/K478D; D12A/E375W/T372R/K478E; D12A/E375W/T372K/K478D; D12A/E375W/T372K/D478E; D12A/E375W/K135D; D12A/E375W/K135E; D12A/E375W/K512D; D12A/E375W/K512E; D12A/E375W/E408K; D12A/E375W/E408R; D12A/E375W/T368D/L480K; D12A/E375W/T368E/L480K; D12A/D456N; N62D/D456N; D12A/D456A; N62D/D456A; D12A/D456S; N62D/D456S; N62D/E375M; N62D/E375L; N62D/E375I; N62D/E375F; N62D/E375D; D12A/K512W; N62D/K512W; D12A/K512Y; N62D/K512Y; D12A/K512F; N62D/K512F; D12A/E375W/K512L; N62D/E375W/K512L; D12A/E375W/K512Y; N52D/E375W/K512Y; D12A/E375W/K512F; N62D/E375W/K512F; D12A/E375Y/K512L; N62D/E375Y/K512L; D12A/E375Y/K512Y; N62D/E375Y/K512Y; D12A/E375Y/K512F; N62D/E375Y/K512F; D12A/E375W/K512H; N62D/E375W/K512H; D12A/E375Y/K512H; N62D/E375Y/K512H; D12A/D510F; N62D/D510F; D12A/D510Y; N62D/D510Y; D12A/D510W; N62D/D510W; D12A/E375W/D510F; N62D/E375W/D510F; D12A/E375W/D510Y; N62D/E375W/D510Y; D12A/E375W/D510W; N62D/E375W/D510W; D12A/E375W/D510W/K512L; N62D/E375W/D510W/K512L; D12A/E375W/D510W/K512F; N62D/E375W/D510W/K512F; D12A/E375W/D510H; N62D/E375W/D510H; D12A/E375W/D510H/K512H; N62D/E375W/D510H/K512H; D12A/E375W/D510H/K512F; N62D/E375W/D510H/K512F; D12A/V509Y; N62D/V509Y; D12A/V509W; N62D/V509W; D12A/V509F; N62D/V509F; D12A/V514Y; N62D/V514Y; D12A/V514W; N62D/V514W; D12A/V514F; N62D/V514F; D12S; D12N; D12Q; D12K; D12A/N62D/Y254F; N62D/Y254V; N62D/Y254A; N62D/Y390F; N62D/Y390A; N62D/S252A; N62D/N387A; N62D/K157E; N62D/I242H; N62D/Y259S; N62D/G320C; N62D/L328V; N62D/T368M; N62D/T368G; N62D/Y369R; N62D/Y369H; N62D/Y369E; N62D/I370V; N62D/I370K; N62D/K371Q; N62D/T372N; N62D/T372D; N62D/T372R; N62D/T372L; N62D/T373A; N62D/T373H; N62D/S374E; N62D/I378K; N62D/K379E; N62D/K379T; N62D/N387D; N62D/Y405V; N62D/L408D; N62D/G413D; N62D/D423V; N62D/I442V; N62D/Y449F; N62D/D456V; N62D/L480M; N62D/V509K; N62D/V509I; N62D/D510A; N62D/V514I; N62D/V514K; N62D/E515K; N62D/D523T; N62D/H149Y/E375W/M554S; M8S/N62D/M102S/H116Y/M188S/E375W; N62D/M97S/E375W; M8S/N62D/M97S/M102S/M188S/E375W/M554S; and M8A/N62D/M97A/M102A/M188A/E375W/M554A. In some instances, a polymerase comprises any one of SEQ ID NOs: 4-10 and a mutation or combination of mutations selected from any one of: K135D, K135E, K512D, K512E, T372D, T372E, L480K, L480R, T368D/L480K, T368E/L480K, T372D/K478R, T372E/K478R, T372R/K478D, T372R/K478E, T372K/K478D, and T372K/K478E. In some instances, a polymerase comprises any one of SEQ ID NOs: 4-10 and a mutation or combination of mutations selected from: M246L, F248L, W367S, Y369V, Y482V, W483S, W483F, W483L, W483V, W483I, W483P, W483Q, H485G, H485N, H485K, H485R, H485A, H485E, H485S, H485I, H485P, H485Q, H485T, H485F, H485L, Y505V, M506L, Y521V, and F526L). In some instances, a polymerase comprises any one of SEQ ID NOs: 4-10 and a mutation or combination of mutations selected from any one of: V250A/E375Y, V250A/E375A/Q380A, V250A/E375C, V250A/E375Y, V250I/E375A/Q380A, V250I/E375C, V250A, V250I, E375A, E375C, E375Y, E375A/Q380A, Q380A, D456N, D456E, D456S, D458N, V250A/E375A/Q380A/D456E, E375Y/V250L, E375Y/V250P, E375Y/V250Q, E375Y/V250R, E375Y/V250Y, E375Y/V250F, E375Y/V250S, E375Y/V250C, E375Y/V250T, E375Y/V250K, E375Y/V250H, E375Y/V250N, E375Y/V250D, E375Y/V250G, E375Y/V250W, E375Y/S388G, E375Y/K512A, E375Y/K525A, Y254V/E375Y, K132A, K383A, K383R, K383P, K371A, K371T, Y254F, Y254V, Y254S, Y254V, Y254S, K379A, K525A, K135A, P255S, S388G, K512A, L384R, E486A, E486D, K478A, E375W, N387A, N387Y, V250A/E375W, D456N/D458N/L351P, Y254V/A377E, D456N/D458N, D169A, D12A/D66A/D169A, T15I, N62D, C22S, C290S, C448S, C530S, C290S/C448S/C530S, C22S/C448S/C530S, C22S/C290S/C530S and C22S/C290S/C448S. In some instances, a polymerase comprises any one of SEQ ID NOs: 4-10 and a mutation or combination of mutations at sites: L253, T368, E375, A484, or K512; E375 or K512; L253, T368 or A484; D193; S215; E420; P477; D66R K135R; K138R; L253T; Y369G; Y369L; L384M; K422A; 1504R; E508K; E508R; D510K; T368/E375 or T368/K512.

In some instances, a polymerase (e.g., Phi29) comprises a sequence of Table 1. In some instances, a polymerase comprises any one of SEQ ID NOs: 11-15. In some instances, a polymerase comprises any one of SEQ ID NOs: 11-15, and at least one mutation. In some instances, a polymerase comprises any one of SEQ ID NOs: 11-15, and at least one substitution. In some instances, a polymerase comprises any one of SEQ ID NOs: 11-15, and at least one addition. In some instances, a polymerase comprises any one of SEQ ID NOs: 11-15, and at least one deletion. In some instances, a polymerase comprises any one of SEQ ID NOs: 11-15 and a substitution at P300. In some instances, a polymerase comprises any one of SEQ ID NOs: 11-15 and substitution P300L. In some instances, a polymerase comprises any one of SEQ ID NOs: 11-15 and a substitution at K512. In some instances, a polymerase comprises any one of SEQ ID NOs: 11-15 and substitution K512A, K512D, K512E, K512W, K512Y, K512F, K512L, or K512H. In some instances, a polymerase comprises any one of SEQ ID NOs: 11-15 and substitution M8R, V51A, M97T, L123S, G197D, K209E, E221K, E239G, Q497P, K512E, E515A, or F526L. In some instances, a polymerase comprises any one of SEQ ID NOs: 11-15 and a mutation or combination of mutations selected from any one of: D12A/E375W/T372D; D12A/E375W/T372E; D12A/E375W/T372R/K478D; D12A/E375W/T372R/K478E; D12A/E375W/T372K/K478D; D12A/E375W/T372K/D478E; D12A/E375W/K135D; D12A/E375W/K135E; D12A/E375W/K512D; D12A/E375W/K512E; D12A/E375W/E408K; D12A/E375W/E408R; D12A/E375W/T368D/L480K; D12A/E375W/T368E/L480K; D12A/D456N; N62D/D456N; D12A/D456A; N62D/D456A; D12A/D456S; N62D/D456S; N62D/E375M; N62D/E375L; N62D/E375I; N62D/E375F; N62D/E375D; D12A/K512W; N62D/K512W; D12A/K512Y; N62D/K512Y; D12A/K512F; N62D/K512F; D12A/E375W/K512L; N62D/E375W/K512L; D12A/E375W/K512Y; N52D/E375W/K512Y; D12A/E375W/K512F; N62D/E375W/K512F; D12A/E375Y/K512L; N62D/E375Y/K512L; D12A/E375Y/K512Y; N62D/E375Y/K512Y; D12A/E375Y/K512F; N62D/E375Y/K512F; D12A/E375W/K512H; N62D/E375W/K512H; D12A/E375Y/K512H; N62D/E375Y/K512H; D12A/D510F; N62D/D510F; D12A/D510Y; N62D/D510Y; D12A/D510W; N62D/D510W; D12A/E375W/D510F; N62D/E375W/D510F; D12A/E375W/D510Y; N62D/E375W/D510Y; D12A/E375W/D510W; N62D/E375W/D510W; D12A/E375W/D510W/K512L; N62D/E375W/D510W/K512L; D12A/E375W/D510W/K512F; N62D/E375W/D510W/K512F; D12A/E375W/D510H; N62D/E375W/D510H; D12A/E375W/D510H/K512H; N62D/E375W/D510H/K512H; D12A/E375W/D510H/K512F; N62D/E375W/D510H/K512F; D12A/V509Y; N62D/V509Y; D12A/V509W; N62D/V509W; D12A/V509F; N62D/V509F; D12A/V514Y; N62D/V514Y; D12A/V514W; N62D/V514W; D12A/V514F; N62D/V514F; D12S; D12N; D12Q; D12K; D12A/N62D/Y254F; N62D/Y254V; N62D/Y254A; N62D/Y390F; N62D/Y390A; N62D/S252A; N62D/N387A; N62D/K157E; N62D/I242H; N62D/Y259S; N62D/G320C; N62D/L328V; N62D/T368M; N62D/T368G; N62D/Y369R; N62D/Y369H; N62D/Y369E; N62D/I370V; N62D/I370K; N62D/K371Q; N62D/T372N; N62D/T372D; N62D/T372R; N62D/T372L; N62D/T373A; N62D/T373H; N62D/S374E; N62D/I378K; N62D/K379E; N62D/K379T; N62D/N387D; N62D/Y405V; N62D/L408D; N62D/G413D; N62D/D423V; N62D/I442V; N62D/Y449F; N62D/D456V; N62D/L480M; N62D/V509K; N62D/V509I; N62D/D510A; N62D/V514I; N62D/V514K; N62D/E515K; N62D/D523T; N62D/H149Y/E375W/M554S; M8S/N62D/M102S/H116Y/M188S/E375W; N62D/M97S/E375W; M8S/N62D/M97S/M102S/M188S/E375W/M554S; and M8A/N62D/M97A/M102A/M188A/E375W/M554A. In some instances, a polymerase comprises any one of SEQ ID NOs: 11-15 and a mutation or combination of mutations selected from any one of: K135D, K135E, K512D, K512E, T372D, T372E, L480K, L480R, T368D/L480K, T368E/L480K, T372D/K478R, T372E/K478R, T372R/K478D, T372R/K478E, T372K/K478D, and T372K/K478E. In some instances, a polymerase comprises any one of SEQ ID NOs: 11-15 and a mutation or combination of mutations selected from: M246L, F248L, W367S, Y369V, Y482V, W483S, W483F, W483L, W483V, W4831, W483P, W483Q, H485G, H485N, H485K, H485R, H485A, H485E, H485S, H485I, H485P, H485Q, H485T, H485F, H485L, Y505V, M506L, Y521V, and F526L). In some instances, a polymerase comprises any one of SEQ ID NOs: 11-15 and a mutation or combination of mutations selected from any one of: V250A/E375Y, V250A/E375A/Q380A, V250A/E375C, V250A/E375Y, V250I/E375A/Q380A, V250I/E375C, V250A, V250I, E375A, E375C, E375Y, E375A/Q380A, Q380A, D456N, D456E, D456S, D458N, V250A/E375A/Q380A/D456E, E375Y/V250L, E375Y/V250P, E375Y/V250Q, E375Y/V250R, E375Y/V250Y, E375Y/V250F, E375Y/V250S, E375Y/V250C, E375Y/V250T, E375Y/V250K, E375Y/V250H, E375Y/V250N, E375Y/V250D, E375Y/V250G, E375Y/V250W, E375Y/S388G, E375Y/K512A, E375Y/K525A, Y254V/E375Y, K132A, K383A, K383R, K383P, K371A, K371T, Y254F, Y254V, Y254S, Y254V, Y254S, K379A, K525A, K135A, P255S, S388G, K512A, L384R, E486A, E486D, K478A, E375W, N387A, N387Y, V250A/E375W, D456N/D458N/L351P, Y254V/A377E, D456N/D458N, D169A, D12A/D66A/D169A, T15I, N62D, C22S, C290S, C448S, C530S, C290S/C448S/C530S, C22S/C448S/C530S, C22S/C290S/C530S and C22S/C290S/C448S. In some instances, a polymerase comprises any one of SEQ ID NOs: 11-15 and a mutation or combination of mutations at sites: L253, T368, E375, A484, or K512; E375 or K512; L253, T368 or A484; D193; S215; E420; P477; D66R K135R; K138R; L253T; Y369G; Y369L; L384M; K422A; I504R; E508K; E508R; D510K; T368/E375 or T368/K512. In some instances, a polymerase comprises at least 90% sequence identity with at least 20 consecutive bases of any one of SEQ ID NOs: 11-15. In some instances, a polymerase comprises at least 80% sequence identity with at least 20 consecutive bases of any one of SEQ ID NOs: 11-15. In some instances, a polymerase comprises at least 70% sequence identity with at least 20 consecutive bases of any one of SEQ ID NOs: 11-15. In some instances, a polymerase comprises at least 90% sequence identity with at least 15 consecutive bases of any one of SEQ ID NOs: 11-15. In some instances, a polymerase comprises at least 80% sequence identity with at least 15 consecutive bases of any one of SEQ ID NOs: 11-15. In some instances, a polymerase comprises at least 70% sequence identity with at least 15 consecutive bases of any one of SEQ ID NOs: 11-15. In some instances, a polymerase comprises at least 90% sequence identity with at least 10 consecutive bases of any one of SEQ ID NOs: 2-10. In some instances, a polymerase comprises at least 80% sequence identity with at least 10 consecutive bases of any one of SEQ ID NOs: 2-10. In some instances, a polymerase comprises at least 70% sequence identity with at least 10 consecutive bases of any one of SEQ ID NOs: 2-10. In some instances, a polymerase comprises at least 80% sequence identity with at least 5 consecutive bases of any one of SEQ ID NOs: 2-10. In some instances, a polymerase comprises at least 80% sequence identity with at least 7 consecutive bases of any one of SEQ ID NOs: 2-10. In some instances, a polymerase comprises at least 90% sequence identity with at least 15 consecutive bases of any one of SEQ ID NOs: 2-10. In some instances, a polymerase comprises at least 80% sequence identity with at least 15 consecutive bases of any one of SEQ ID NOs: 2-10.

Polymerase variants described herein may possess increased processivity relative to a polymerase of SEQ ID NO: 1. In some instances, this is described as a number of bases (nt) per minute. In some instances, a polymerase described herein incorporates at least 2000 nt/min at 30 degrees C. using a single-stranded M13 template. In some instances, a polymerase described herein incorporates at least 2000 nt/min, 2200 nt/min, 2500 nt/min, 2700 nt/min or at least 3000 nt/min at 30 degrees C. using a single-stranded M13 template. In some instances, a polymerase described herein incorporates at least 1500 nt/min, 2000 nt/min, 2200 nt/min, 2500 nt/min, 2700 nt/min or at least 3000 nt/min at 30 degrees C. using a single-stranded M13 template, in the presence of nucleotides comprising at least 1% dideoxynucleotides. In some instances, a polymerase described herein incorporates at least 1500 nt/min, 2000 nt/min, 2200 nt/min, 2500 nt/min, 2700 nt/min or at least 3000 nt/min at 30 degrees C. using a single-stranded M13 template, in the presence of nucleotides comprising at least 5% dideoxynucleotides. In some instances, a polymerase described herein incorporates at least 1500 nt/min, 2000 nt/min, 2200 nt/min, 2500 nt/min, 2700 nt/min or at least 3000 nt/min at 30 degrees C. using a single-stranded M13 template, in the presence of nucleotides comprising at least 10% dideoxynucleotides.

Polymerase variants described herein may possess increased strand displacement activity relative to a polymerase of SEQ ID NO: 1. In some instances, strand displacement activity is measured using a replication slippage assay (Canceill, et al. J. Biol. Chem. 1999, 27481). In some instances, polymerases described herein comprise 5%, 10%, 15%, 20%, 30%, 40%, 50%, 60%, 70%, 80%, or 90% less replication slippage than a polymerase of SEQ ID NO: 1. In some instances, polymerases described herein comprise 5-90%, 10-90%, 25-90%, 50-95%, 50-99%, 5-25%, or 5-50% less replication slippage than a polymerase of SEQ ID NO: 1. In some instances, polymerases described herein comprise 5%, 10%, 15%, 20%, 30%, 40%, 50%, 60%, 70%, 80%, or 90% less replication slippage than a polymerase of SEQ ID NO: 1 in the presence of nucleotides comprising at least 10% dideoxynucleotides. In some instances, polymerases described herein comprise 5-90%, 10-90%, 25-90%, 50-95%, 50-99%, 5-25%, or 5-50% less replication slippage than a polymerase of SEQ ID NO: 1 in the presence of nucleotides comprising 5-20% dideoxynucleotides. In some instances, polymerases described herein comprise 5%, 10%, 15%, 20%, 30%, 40%, 50%, 60%, 70%, 80%, or 90% less replication slippage than a polymerase of SEQ ID NO: 1 in the presence of nucleotides comprising at least 5% dideoxynucleotides. In some instances, polymerases described herein comprise 5%, 10%, 15%, 20%, 30%, 40%, 50%, 60%, 70%, 80%, or 90% less replication slippage than a polymerase of SEQ ID NO: 1 in the presence of nucleotides comprising at least 1% dideoxynucleotides.

Polymerase variants described herein may possess increased template binding relative to a polymerase of SEQ ID NO: 1. In some instances, polymerases described herein comprise at least 5%, 10%, 20%, 30%, 40%, 50%, 80%, 90%, 100%, 200%, or 500% increase in K_{D} value for a template relative to a polymerase of SEQ ID NO: 1. In some instances, polymerases described herein comprise a 50-400%, 10-90%, 25-90%, 50-100%, 50-200%, 50-250%, or 50-500% increase in K_{D} value for a template relative to a polymerase of SEQ ID NO: 1.

Polymerase variants described herein may possess increased primer binding relative to a polymerase of SEQ ID NO: 1. In some instances, polymerases described herein comprise at least 5%, 10%, 20%, 30%, 40%, 50%, 80%, 90%, 100%, 200%, or 500% increase in K_{D} value for a primer relative to a polymerase of SEQ ID NO: 1. In some instances, polymerases described herein comprise a 50-400%, 10-90%, 25-90%, 50-100%, 50-200%, 50-250%, or 50-500% increase in K_{D} value for a primer relative to a polymerase of SEQ ID NO: 1.

Polymerase variants described herein may possess a decreased error rate relative to a polymerase of SEQ ID NO: 1. In some instances, a polymerase described herein comprises an error rate of less than 1×10⁻⁶, 2×10⁻⁶, 5×10⁻⁶, 8×10⁻⁶, 1×10⁻⁷, 2×10⁻⁷, 5×10⁻⁷, 8×10⁻⁷, 1×10⁻⁸, 2×10⁻⁸, 5×10⁻⁸, or less than 8×10⁻⁸. In some instances, a polymerase described herein comprises an error rate of 1×10⁻⁶ to 8×10⁻⁸, 2×10⁻⁶ to 8×10⁻⁷, 5×10⁻⁶to 5×10⁻⁷, 1×10⁻⁶ to 8×10⁻⁷, or 5×10⁻⁶ to 8×10⁻⁸. Polymerase variants described herein may possess increased 3′->5′ exonuclease activity relative to a polymerase of SEQ ID NO: 1. In some instances, polymerases described herein comprise at least 5%, 10%, 20%, 30%, 40%, 50%, 80%, 90%, 100%, 200%, or 500% increase in exonuclease activity relative to a polymerase of SEQ ID NO: 1. In some instances, polymerases described herein comprise a 50-400%, 10-90%, 25-90%, 50-100%, 50-200%, 50-250%, or 50-500% increase in exonuclease activity relative to a polymerase of SEQ ID NO: 1.

Polymerase variants described herein may possess altered affinity (selectivity) for thymine/alanine vs. guanidine/cytosine nucleotides. In some instances, polymerases described herein comprise at least 5%, 10%, 20%, 30%, 40%, 50%, 80%, 90%, 100%, 200%, or 500% increase in TA:GC affinity relative to a polymerase of SEQ ID NO: 1. In some instances, polymerases described herein comprise at least 5%, 10%, 20%, 30%, 40%, 50%, 80%, 90%, 100%, 200%, or 500% increase in GC:TA affinity relative to a polymerase of SEQ ID NO: 1. In some instances, polymerases described herein comprise a 50-400%, 10-90%, 25-90%, 50-100%, 50-200%, 50-250%, or 50-500% increase in GC:TA affinity relative to a polymerase of SEQ ID NO: 1.

Polymerase variants described herein may possess altered affinity (selectivity) for dideoxynucleotides. In some instances, polymerases described herein comprise at least 5%, 10%, 20%, 30%, 40%, 50%, 80%, 90%, 100%, 200%, or 500% increase in dideoxynucleotide affinity relative to a polymerase of SEQ ID NO: 1. In some instances, polymerases described herein comprise a 50-400%, 10-90%, 25-90%, 50-100%, 50-200%, 50-250%, or 50-500% increase in dideoxynucleotide affinity relative to a polymerase of SEQ ID NO: 1. Polymerases described herein, e.g., variant polymerases, may incorporate dideoxynucleotides more efficiently, which results in shorter amplification products relative to a wild-type polymerase (e.g., Phi29 polymerase). In some instances, polymerases described herein generate amplification products at least 1%, 2%, 5%, 10%, 15%, 20%, 30%, 50%, 75%, 90%, 150%, 300%, or at least 500% smaller in length than a wild-type polymerase, in the presence of nucleotides comprising at least 1% dideoxynucleotides. In some instances, polymerases described herein generate amplification products at least 1%, 2%, 5%, 10%, 15%, 20%, 30%, 50%, 75%, 90%, 150%, 300%, or at least 500% smaller in length than a wild-type polymerase, in the presence of nucleotides comprising at least 5% dideoxynucleotides. In some instances, polymerases described herein generate amplification products at least 1%, 2%, 5%, 10%, 15%, 20%, 30%, 50%, 75%, 90%, 150%, 300%, or at least 500% smaller in length than a wild-type polymerase, in the presence of nucleotides comprising at least 10% dideoxynucleotides. In some instances, polymerases described herein generate amplification products at least 1%, 2%, 5%, 10%, 15%, 20%, 30%, 50%, 75%, 90%, 150%, 300%, or at least 500% smaller in length than a wild-type polymerase, in the presence of nucleotides comprising 1-10% dideoxynucleotides. In some instances, polymerases described herein generate amplification products at least 1%, 2%, 5%, 10%, 15%, 20%, 30%, 50%, 75%, 90%, 150%, 300%, or at least 500% smaller in length than a wild-type polymerase, in the presence of nucleotides comprising 5-20% dideoxynucleotides.

Polymerase variants described herein may possess increased temperature stability. In some instances, a polymerase variant maintains at least 99% activity after exposure to 65 degrees C. for 10 minutes. In some instances, a polymerase variant maintains 90-99% activity after exposure to 65 degrees C. for 10 minutes. In some instances, a polymerase variant maintains 80-99% activity after exposure to 65 degrees C. for 10 minutes. In some instances, a polymerase variant maintains 50-99% activity after exposure to 65 degrees C. for 10 minutes. In some instances, a polymerase variant maintains at least 99% activity after exposure to 65 degrees C. for 10 minutes. In some instances, a polymerase variant maintains at least 90% activity after exposure to 65 degrees C. for 10 minutes. In some instances, a polymerase variant maintains at least 80% activity after exposure to 65 degrees C. for 10 minutes. In some instances, a polymerase variant maintains at least 50% activity after exposure to 65 degrees C. for 10 minutes. In some instances, a polymerase variant maintains at least 30% activity after exposure to 65 degrees C. for 10 minutes.

Methods and Applications

Described herein are methods of identifying mutations in cells with the methods of PTA. Use of the PTA method in some instances results in improvements over known methods, for example, MDA. PTA in some instances has lower false positive and false negative variant calling rates than the MDA method. Genomes, such as NA12878 platinum genomes, are in some instances used to determine if the greater genome coverage and uniformity of PTA would result in lower false negative variant calling rate. Without being bound by theory, it may be determined that the lack of error propagation in PTA decreases the false positive variant call rate. The amplification balance between alleles with the two methods is in some cases estimated by comparing the allele frequencies of the heterozygous mutation calls at known positive loci. In some instances, amplicon libraries generated using PTA are further amplified by PCR. In some instances, the PTA method identifies mutations present in single cells of a population, wherein a mutation detected by PTA occurs in less than 2%, 1%, 0.5%, 0.2%, 0.1%, 0.05%, 0.02%, 0.01%, 0.001%, 0.0001%, or less than 0.00001% of the cells in the population. In some instances, the PTA method identifies mutations in less than 2%, 1%, 0.5%, 0.2%, 0.1%, 0.05%, 0.02%, 0.01%, 0.001%, 0.0001%, or less than 0.00001% of the sequencing reads for a given base or region.

Gene Editing Safety

The continued development of genome editing tools shows great promise for improving human health; from correcting genes that result in or contribute to the formation of disease (such as sickle cell anemia, and many other diseases) to the eradication of infectious diseases that are currently incurable. However, the safety of these interventions remain unclear as a result of our incomplete understanding of how these tools interact with and permanently alter other locations in the genomes of edited cells. Methods have been developed to estimate the off-target rates of genome editing strategies, but tools that have been developed to date interrogate groups of cells together, resulting in the inability to measure the per cell off-target rates and variance in off-target activity between cells, as well as to detect rare editing events that occur in a small number of cells. These suboptimal strategies for measuring genome editing fidelity have resulted in a limited capacity to determine the sensitivity and specificity of a given genome editing approach.

Gene therapy methods may comprise modification of a mutated, disease causing gene, knockout of a disease causing gene, or introduction of a new gene in cells. Such approaches in some instances comprise modification of genomic DNA. In other instances, viral or other delivery systems are configured such that they do not integrate or modify genomic DNA in cells. However, such systems may nevertheless produce unwanted or unexpected modifications to somatic or germline DNA. Taking advantage of the improved variant calling sensitivity and specificity of PTA in single cells, quantitative measurements of unintended insertion rates of gene therapy approaches with high sensitivity in single cells in some instances is conducted. The method is some cases detects the insertion of specific sequences in a non-desired location by detecting the surrounding sequence to determine if the gene therapy approach is causes insertion or modification of the host genome.

Described herein are methods of identifying mutations and structural modifications (i.e. translocation, insertions and deletions) in animal, plant or microbial cells that have undergone genome editing (e.g., CRISPR (Clustered regularly interspaced short palindromic repeats), TALEN (Transcription activator-like effector nucleases), ZFN (Zinc finger nucleases), recombinase, meganucleases, or other genome editing technologies). In some instances, genome editing comprises site-specific or targeted genome editing. Such cells in some instances can be isolated and subjected to PTA and sequencing to determine mutation burden, mutation combination and structural variation in each cell. The per-cell mutation rate and locations of mutations that result from a genome editing protocol are in some instances used to assess the safety and/or efficiency of a given genome editing method. Identification of mutations in some instances comprises comparing sequencing data obtained using the PTA method with a reference sequence. In some instances, the reference sequence is a genome. In some instances, at least one mutation is identified by PTA after a gene editing process. In some instances, the reference sequence is a specificity-determining sequence which promotes introduction of a mutation into a target sequence of a nucleic acid. In some instances, at least one mutation is identified by PTA after a gene editing process, wherein the mutation is located in the target sequence. In some instances, off-target mutation rates are analyzed by identifying at least one mutation not in the target sequence. Although some areas of a nucleic acid may be predicted to suffer off-target mutation based on sequence homology to target sequences, regions with lower homology may also have off-target mutations. In some instances, the PTA method identifies a mutation in an off-target region of a sequence comprising at least 0, 1, 2, 3, 4, 5, 6, 7, or 8 base mismatches with the target sequence or reverse complement thereof. In some instances, single cells are analyzed with PTA. In some instances, populations of cells are analyzed with PTA.

Many current methods of mutational analysis obtain sequencing data on bulk cell populations. However, such approaches provide limited information regarding the actual frequency of mutations in the population, Single cell analysis using PTA in some instances provides much higher resolution of the off target rate of insertion, strand breaks (resulting in mutation), and translocation as the number of cells (i.e. a single cell) is known. PTA, which has a known rate of variation detection, in a known number of single cells, allows the method in some instances to accurately determine the per cell frequency and combinations of alterations in a population of cells. In some instances, at least 10, 100, 1000, 10,000, 100,000, or more than 100,000 single cells are analyzed with PTA to establish a rate of variation. In some instances, no more than 10, 100, 1000, 10,000, 100,000, or no more than 100,000 single cells are analyzed with PTA to establish a rate of variation. In some instances, 10-1000, 50-5000, 100-100,000, 1000-100,000, 100-1,000,000, or 100-10,000 single cells are analyzed with PTA to establish a rate of variation. In some instances, mutations identified by analysis of one or more single cells are not identified or detected from bulk sequencing of the population of cells.

CRISPR may be used to introduce mutations into one or more cells, such as mammalian cells which are then analyzed by PTA. In some instances, the specificity-determining sequence is present in a CRISPR RNA (crRNA) or single guide RNA (sgRNA). In some instances, the mammalian cells are human cells. In some instances, the cells originate from liver, skin, kidney, blood, or lung. In some instances, the cells are primary cells. In some instances, the cells are stem cells. Previously reported methods of identifying off-target mutations generated from CRISPR have included pulldown of sequences binding to catalytically active Cas9, however this may lead to false positives as mutations are not introduced at all Cas9 binding sites. In some instances, the PTA method identifies at least one mutation present in a region of a sequence which binds to catalytically active Cas9. In some instances, the PTA method results in fewer false positives for at least one mutation present in a region of a sequence which binds to catalytically active Cas9.

Described herein are methods of identifying mutations in animal, plant or microbial cells that have undergone genome editing (e.g., CRISPR, TALEN, ZFN, recombinase, meganucleases, or other technologies), wherein the method comprises amplification of a genomic or fragment thereof in the presence of at least one terminator nucleotide. In some instances, amplification with the terminator takes place in solution. In some instances, one of either at least one primer or at least one genomic fragment is attached to a surface. In some instances, at least one primer is attached to a first solid support, and at least one genomic fragment is attached to a second solid support, wherein the first solid support and the second solid support are not connected. In some instances, at least one primer is attached to a first solid support, and at least one genomic fragment is attached to a second solid support, wherein the first solid support and the second solid support are not the same solid support. In some instances, the method comprises amplification of a genomic or fragment thereof in the presence of at least one terminator nucleotide, wherein the number of amplification cycles is less than 12, 10, 9, 8, 7, 6, 5, 4, or less than 3 cycles. In some instances, the average length of amplification products is 100-1000, 200-500, 200-700, 300-700, 400-1000, or 500-1200 bases in length. In some instances, the method comprises amplification of a genomic or fragment thereof in the presence of at least one terminator nucleotide, wherein the number of amplification cycles is no more than 6 cycles. In some instances, the at least one terminator nucleotide does comprise a detectable label or tag. In some instances, the amplification comprises 2, 3, or 4 terminator nucleotides. In some instances, at least two of the terminator nucleotides comprise a different base. In some instances, at least three of the terminator nucleotides comprise a different base. In some instances, four terminator nucleotides each comprise a different base. The number of direct copies may be controlled in some instances by the number of amplification cycles. In some instances, no more than 30, 25, 20, 15, 13, 11, 10, 9, 8, 7, 6, 5, 4, or 3 cycles are used to generate copies of the target nucleic acid molecule. In some instances, about 30, 25, 20, 15, 13, 11, 10, 9, 8, 7, 6, 5, 4, or about 3 cycles are used to generate copies of the target nucleic acid molecule. In some instances, 3, 4, 5, 6, 7, or 8 cycles are used to generate copies of the target nucleic acid molecule. In some instances, 2-4, 2-5, 2-7, 2-8, 2-10, 2-15, 3-5, 3-10, 3-15, 4-10, 4-15, 5-10 or 5-15 cycles are used to generate copies of the target nucleic acid molecule. Amplicon libraries generated using the methods described herein are in some instances subjected to additional steps, such as adapter ligation and further amplification. In some instances, such additional steps precede a sequencing step. In some instances, the cycles are PCR cycles. In some instances, the cycles represent annealing, extension, and denaturation. In some instances, the cycles represent annealing, extension, and denaturation which occur under isothermal or essentially isothermal conditions.

Described herein are methods for determining the safety of gene therapies. In some instances, the functions of a cell are modified through a gene editing or other expression method. In some instances, viral delivery systems to change cellular functions are configured such that they do not integrate into the genome of the cell. In some instances the PTA method is used to identify unexpected or unwanted changes to cell genomes. In some instances, PTA is used to identify mutations to somatic or germline DNA that result from gene therapy.

Clonal Analysis of Tumor Cells

Cells analyzed using the methods described herein in some instances comprise tumor cells. For example, circulating tumor cells can be isolated from a fluid taken from patients, such as but not limited to, blood, bone marrow, urine, saliva, cerebrospinal fluid, pleural fluid, pericardial fluid, ascites, or aqueous humor. The cells are then subjected to the methods described herein (e.g. PTA) and sequencing to determine mutation burden and mutation combination in each cell. These data are in some instances used for the diagnosis of a specific disease or as tools to predict treatment response. Similarly, in some instances cells of unknown malignant potential in some instances are isolated from fluid taken from patients, such as but not limited to, blood, bone marrow, urine, saliva, cerebrospinal fluid, pleural fluid, pericardial fluid, ascites, or aqueous humor. After utilizing the methods described herein and sequencing, such methods are further used to determine mutation burden and mutation combination in each cell. These data are in some instances used for the diagnosis of a specific disease or as tools to predict progression of a premalignant state to overt malignancy. In some instances, cells can be isolated from primary tumor samples. The cells can then undergo PTA and sequencing to determine mutation burden and mutation combination in each cell. These data can be used for the diagnosis of a specific disease or are as tools to predict the probability that a patient's malignancy is resistant to available anti-cancer drugs. By exposing samples to different chemotherapy agents, it has been found that the major and minor clones have differential sensitivity to specific drugs that does not necessarily correlate with the presence of a known “driver mutation,” suggesting that combinations of mutations within a clonal population determine its sensitivities to specific chemotherapy drugs. Without being bound by theory, these findings suggest that a malignancy may be easier to eradicate if premalignant lesions that have not yet expanded are and evolved into clones are detected whose increased number of genome modification may make them more likely to be resistant to treatment. See, Ma et al., 2018, “Pan-cancer genome and transcriptome analyses of 1,699 pediatric leukemias and solid tumors.” A single-cell genomics protocol is in some instances used to detect the combinations of somatic genetic variants in a single cancer cell, or clonotype, within a mixture of normal and malignant cells that are isolated from patient samples. This technology is in some instances further utilized to identify clonotypes that undergo positive selection after exposure to drugs, both in vitro and/or in patients. By comparing the surviving clones exposed to chemotherapy compared to the clones identified at diagnosis, a catalog of cancer clonotypes can be created that documents their resistance to specific drugs. PTA methods in some instances detect the sensitivity of specific clones in a sample composed of multiple clonotypes to existing or novel drugs, as well as combinations thereof, where the method can detect the sensitivity of specific clones to the drug. This approach in some instances shows efficacy of a drug for a specific clone that may not be detected with current drug sensitivity measurements that consider the sensitivity of all cancer clones together in one measurement. When the PTA described herein are applied to patient samples collected at the time of diagnosis in order to detect the cancer clonotypes in a given patient's cancer, a catalog of drug sensitivities may then be used to look up those clones and thereby inform oncologists as to which drug or combination of drugs will not work and which drug or combination of drugs is most likely to be efficacious against that patient's cancer.

Clinical and Environmental Mutagenesis

Described herein are methods of measuring the mutagenicity of an environmental factor. For example, cells (single or a population) are exposed to a potential environmental condition. For example, cells such originating from organs (liver, pancreas, lung, colon, thyroid, or other organ), tissues (skin, or other tissue), blood, or other biological source are in some instances used with the method. In some instances, an environmental condition comprises heat, light (e.g. ultraviolet), radiation, a chemical substance, or any combination thereof. After an amount of exposure to the environmental condition, in some instances minutes, hours, days, or longer, single cells are isolated and subjected to the PTA method. In some instances, molecular barcodes and unique molecular identifiers are used to tag the sample. The sample is sequenced and then analyzed to identify mutations resulting from exposure to the environmental condition. In some instances, such mutations are compared with a control environmental condition, such as a known non-mutagenic substance, vehicle/solvent, or lack of an environmental condition. Such analysis in some instances not only provides the total number of mutations caused by the environmental condition, but also the locations and nature of such mutations. Patterns are in some instances identified from the data, and may be used for diagnosis of diseases or conditions. In some instances, patterns are used to predict future disease states or conditions. In some instances, the methods described herein measure the mutation burden, locations, and patterns in a cell after exposure to an environmental agent, such as, e.g., a potential mutagen or teratogen. This approach in some instances is used to evaluate the safety of a given agent, including its potential to induce mutations that can contribute to the development of a disease. For example, the method could be used to predict the carcinogenicity or teratogenicity of an agent to specific cell types after exposure to a specific concentration of the specific agent. In some instances, the agent is a medicine or drug. In some instances, the agent is a food. In some instances, the agent is a genetically modified food. In some instances, the agent is a pesticide or other agricultural chemical. In some instances, the location and rate of mutations is used to predict the age of an organism. Such methods are in some instances performed on samples that are hundreds, thousands, or tens of thousands of years old. Mutational patterns are in some cases compared with other data methods such as carbon dating to generate standard curves. In some instances the age of a human is determined by comparison of mutational numbers and patterns from a sample.

Described herein are methods of determining mutations in cells that are used for cellular therapy, such as but not limited to the transplantation of induced pluripotent stem cells, transplantation of hematopoietic or other cells that have not be manipulated, or transplantation of hematopoietic or other cells that have undergone genome edits. The cells can then undergo PTA and sequencing to determine mutation burden and mutation combination in each cell. The per-cell mutation rate and locations of mutations in the cellular therapy product can be used to assess the safety and potential efficacy of the product, including measurement of neoantigen burden.

Microbial Samples

Described herein are methods of analyzing microbial samples. In another embodiment, microbial cells (e.g., bacteria, fungi, protozoa) can be isolated from plants or animals (e.g., from microbiota samples [e.g., GI microbiota, skin microbiota, etc.] or from bodily fluids such as, e.g., blood, bone marrow, urine, saliva, cerebrospinal fluid, pleural fluid, pericardial fluid, ascites, or aqueous humor). In addition, microbial cells may be isolated from indwelling medical devices, such as but not limited to, intravenous catheters, urethral catheters, cerebrospinal shunts, prosthetic valves, artificial joints, or endotracheal tubes. The cells can then undergo PTA and sequencing to determine the identity of a specific microbe, as well as to detect the presence of microbial genetic variants that predict response (or resistance) to specific antimicrobial agents. These data can be used for the diagnosis of a specific infectious disease and/or as tools to predict treatment response. In some instances, single microbial cells are analyzed for mutations. In one embodiment, PTA is used to identify microorganisms with high value for industrial applications, such as production of biofuels or environmental restoration (oil spill cleanup, CO₂ sequestration/removal). In some instances, microbial samples are obtained from extreme environments, such as deep sea vents, ocean, mines, streams, lakes, meteorites, glaciers, or volcanoes. In some instances, microbial samples comprise strains of microbes that are “unculturable” in the laboratory under standard conditions.

Fetal Cells

In a further embodiment, cells can be isolated from blastomeres that are created by in vitro fertilization. The cells can then undergo PTA and sequencing to determine the burden and combination of potentially disease predisposing genetic variants in each cell. The mutation profile of the cell can then be used to extrapolate the genetic predisposition of the blastomere to specific diseases prior to implantation.

In some instances, the methods (e.g., PTA) described herein result in higher detection sensitivity and/or lower rates of false positives for the detection of mutations. In some instances, PTA results in higher detection sensitivity and/or lower rates of false positives for the detection of mutations when compared to methods such as in-silico prediction, ChIP-seq, GUIDE-seq, circle-seq, HTGTS (High-Throughput Genome-Wide Translocation Sequencing), DLV (integration-deficient lentivirus), Digenome-seq, FISH (fluorescence in situ hybridization), or DISCOVER-seq.

Single Cell Analysis

Described herein are methods and compositions for analysis of single cells. Analysis of cells in bulk provides general information about the cell population, but often is unable to detect low-frequency mutants over the background. Such mutants may comprise important properties such as drug resistance or mutations associated with cancer. In some instances, DNA, RNA, and/or proteins from the same single cell are analyzed in parallel. The analysis may include identification of epigenetic post-translational (e.g., glycosylation, phosphorylation, acetylation, ubiquination, histone modification) and/or post-transcriptional (e.g., methylation, hydroxymethylation) modifications. Such methods may comprise “Primary Template-Directed Amplification” (PTA) to obtain libraries of nucleic acids for sequencing. In some instances PTA is combined with additional steps or methods such as RT-PCR or proteome/protein quantification techniques (e.g., mass spectrometry, antibody staining, etc.). In some instances, various components of a cell are physically or spatially separated from each other during individual analysis steps. For example, a workflow in some instances comprises the general steps of labeling proteins, generating mRNA, generating RT-PCR libraries, isolating genomic DNA, subjecting the genomic DNA to PTA, generating a gDNA library, and sequencing the two libraries. Proteins are first labeled with antibodies and sorted based on fluorescent markers. After RT-PCR, first strand mRNA products are generated and then removed for analysis. Libraries are then generated from RT-PCR products and barcodes present on protein-specific antibodies, which are subsequently sequenced. In parallel, genomic DNA from the same cell is subjected to PTA, a library generated, and sequenced. Sequencing results from the genome, proteome, and transcriptome are in some instances pooled using bioinformatics methods. Methods described herein in some instances comprise any combination of labeling, cell sorting, affinity separation/purification, lysing of specific cell components (e.g., outer membrane, nucleus, etc.), RNA amplification, DNA amplification (e.g., PTA), or other step associated with protein, RNA, or DNA isolation or analysis.

Described herein is a first method of single cell analysis comprising analysis of RNA and DNA from a single cell. In some instances, the method comprises isolation of single cells, lysis of single cells, and reverse transcription (RT). In some instances, reverse transcription is carried out with template switching oligonucleotides (TSOs). In some instances, TSOs comprise a molecular TAG such as biotin, which allows subsequent pull-down of cDNA RT products, and PCR amplification of RT products to generate a cDNA library. Alternatively or in combination, centrifugation is used to separate RNA in the supernatant from cDNA in the cell pellet. Remaining cDNA is in some instances fragmented and removed with UDG (uracil DNA glycosylase), and alkaline lysis is used to degrade RNA and denature the genome. After neutralization, addition of primers and PTA, amplification products are in some instances purified on SPRI (solid phase reversible immobilization) beads, and ligated to adapters to generate a gDNA library.

Described herein is a second method of single cell analysis comprising analysis of RNA and DNA from a single cell. In some instances, the method comprises isolation of single cells, lysis of single cells, and reverse transcription (RT). In some instances, reverse transcription is carried out with template switching oligonucleotides (TSOs). In some instances, TSOs comprise a molecular TAG such as biotin, which allows subsequent pull-down of cDNA RT products, and PCR amplification of RT products to generate a cDNA library. In some instances, alkaline lysis is then used to degrade RNA and denature the genome. After neutralization, addition of random primers and PTA, amplification products are in some instances purified on SPRI (solid phase reversible immobilization) beads, and ligated to adapters to generate a gDNA library. RT products are in some instances isolated by pulldown, such as a pulldown with streptavidin beads.

Described herein is a third method of single cell analysis comprising analysis of RNA and DNA from a single cell. In some instances, the method comprises isolation of single cells, lysis of single cells, and reverse transcription (RT). In some instances, reverse transcription is carried out with template switching oligonucleotides (TSOs) in the presence of terminator nucleotides. In some instances, TSOs comprise a molecular TAG such as biotin, which allows subsequent pull-down of cDNA RT products, and PCR amplification of RT products to generate a cDNA library. In some instances, alkaline lysis is then used to degrade RNA and denature the genome. After neutralization, addition of random primers and PTA, amplification products are in some instances purified on SPRI (solid phase reversible immobilization) beads, and ligated to adapters to generate a DNA library. RT products are in some instances isolated by pulldown, such as a pulldown with streptavidin beads.

Described herein is a fourth method of single cell analysis comprising analysis of RNA and DNA from a single cell. In some instances, the method comprises isolation of single cells, lysis of single cells, and reverse transcription (RT). In some instances, reverse transcription is carried out with template switching oligonucleotides (TSOs). In some instances, TSOs comprise a molecular TAG such as biotin, which allows subsequent pull-down of cDNA RT products, and PCR amplification of RT products to generate a cDNA library. In some instances, alkaline lysis is then used to degrade RNA and denature the genome. After neutralization, addition of random primers and PTA, amplification products are in some instances subjected to RNase and cDNA amplification using blocked and labeled primers. gDNA is purified on SPRI (solid phase reversible immobilization) beads, and ligated to adapters to generate a gDNA library. RT products are in some instances are isolated by pulldown, such as a pulldown with streptavidin beads.

Described herein is a fifth method of single cell analysis comprising analysis of RNA and DNA from a single cell. A population of cells is contacted with an antibody library, wherein antibodies are labeled. In some instances, antibodies are labeled with either fluorescent labels, nucleic acid barcodes, or both. Labeled antibodies bind to at least one cell in the population, and such cells are sorted, placing one cell per container (e.g., a tube, vial, microwell, etc.). In some instances, the container comprises a solvent. In some instances, a region of a surface of a container is coated with a capture moiety. In some instances, the capture moiety is a small molecule, an antibody, a protein, or other agent capable of binding to one or more cells, organelles, or other cell component. In some instances, at least one cell, or a single cell, or component thereof, binds to a region of the container surface. In some instances, a nucleus binds to the region of the container. In some instances, the outer membrane of the cell is lysed, releasing mRNA into a solution in the container. In some instances, the nucleus of the cell containing genomic DNA is bound to a region of the container surface. Next, RT is often performed using the mRNA in solution as a template to generate cDNA. In some instances, template switching primers comprise from 5′ to 3′ a TSS region (transcription start site), an anchor region, a RNA BC region, and a poly dT tail. In some instances, the poly dT tail binds to poly A tail of one or more mRNAs. In some instances, template switching primers comprise from 3′ to 5′ a TSS region, an anchor region, and a poly G region. In some instances, the poly G region comprises riboG. In some instances the poly G region binds to a poly C region on an mRNA transcript. In some instances, riboG was added to the mRNA transcripts by a terminal transferase. After removal of RT PCR products for subsequent sequencing, any remaining RNA in the cell is removed by UNG. The nucleus is then lysed, and the released genomic DNA is subjected to the PTA method using random primers with an isothermal polymerase. In some instances, primers are 6-9 bases in length. In some instances, PTA generates genomic amplicons of 250-1500 bases in length. In some instances, the methods described herein generate a short fragment cDNA pool with about 500, about 750, about 1000, about 5000, or about 10,000 fold amplification. In some instances, the methods described herein generate a short fragment cDNA pool with 500-5000, 750-1500, or 250-10,000 fold amplification. PTA products are optionally subjected to additional amplification and sequenced.

Sample Preparation and Isolation of Single Cells

Methods described herein may require isolation of single cells for analysis. Any method of single cell isolation may be used with PTA, such as mouth pipetting, micro pipetting, flow cytometry/FACS, microfluidics, methods of sorting nuclei (tetraploid or other), or manual dilution. Such methods are aided by additional reagents and steps, for example, antibody-based enrichment (e.g., circulating tumor cells), other small-molecule or protein-based enrichment methods, or fluorescent labeling. In some instances, a method of multiomic analysis described herein comprises mechanical or enzymatic dissociate of cells from larger tissues.

Preparation and Analysis of Cell Components

Methods of multiomic analysis comprising PTA described herein may comprise one or more methods of processing cell components such as DNA, RNA, and/or proteins. In some instances, the nucleus (comprising genomic DNA) is physically separated from the cytosol (comprising mRNA), followed by a membrane-selective lysis buffer to dissolve the membrane but keep the nucleus intact. The cytosol is then separated from the nucleus using methods including micro pipetting, centrifugation, or anti-body conjugated magnetic microbeads. In another instance, an oligo-dT primer coated magnetic bead binds polyadenylated mRNA for separation from DNA. In another instance, DNA and RNA are preamplified simultaneously, and then separated for analysis. In another instance, a single cell is split into two equal pieces, with mRNA from one half processed, and genomic DNA from the other half processed.

Multiomics

Methods described herein (e.g., PTA) may be used as a replacement for any number of other known methods in the art which are used for single cell sequencing (multiomics or the like). PTA may substitute genomic DNA sequencing methods such as MDA, PicoPlex, DOP-PCR, MALBAC, or target-specific amplifications. In some instances, PTA replaces the standard genomic DNA sequencing method in a multiomics method including DR-seq (Dey et al., 2015), G&T seq (MacAulay et al., 2015), scMT-seq (Hu et al., 2016), sc-GEM (Cheow et al., 2016), scTrio-seq (Hou et al., 2016), simultaneous multiplexed measurement of RNA and proteins (Darmanis et al., 2016), scCOOL-seq (Guo et al., 2017), CITE-seq (Stoeckius et al., 2017), REAP-seq (Peterson et al., 2017), scNMT-seq (Clark et al., 2018), or SIDR-seq (Han et al., 2018). In some instances, a method described herein comprises PTA and a method of polyadenylated mRNA transcripts. In some instances, a method described herein comprises PTA and a method of non-polyadenylated mRNA transcripts. In some instances, a method described herein comprises PTA and a method of total (polyadenylated and non-polyadenylated) mRNA transcripts.

In some instances, PTA is combined with a standard RNA sequencing method to obtain genome and transcriptome data. In some instances, a multiomics method described herein comprises PTA and one of the following: Drop-seq (Macosko, et al. 2015), mRNA-seq (Tang et al., 2009), InDrop (Klein et al., 2015), MARS-seq (Jaitin et al., 2014), Smart-seq2 (Hashimshony, et al., 2012; Fish et al., 2016), CEL-seq (Jaitin et al., 2014), STRT-seq (Islam, et al., 2011), Quartz-seq (Sasagawa et al., 2013), CEL-seq2 (Hashimshony, et al. 2016), cytoSeq (Fan et al., 2015), SuPeR-seq (Fan et al., 2011), RamDA-seq (Hayashi, et al. 2018), MATQ-seq (Sheng et al., 2017), or SMARTer (Verboom et al., 2019).

Various reaction conditions and mixes may be used for generating cDNA libraries for transcriptome analysis. In some instances, an RT reaction mix is used to generate a cDNA library. In some instances, the RT reaction mixture comprises a crowding reagent, at least one primer, a template switching oligonucleotide (TSO), a reverse transcriptase, and a dNTP mix. In some instances, an RT reaction mix comprises an RNAse inhibitor. In some instances an RT reaction mix comprises one or more surfactants. In some instances an RT reaction mix comprises Tween-20 and/or Triton-X. In some instances an RT reaction mix comprises Betaine. In some instances an RT reaction mix comprises one or more salts. In some instances an RT reaction mix comprises a magnesium salt (e.g., magnesium chloride) and/or tetramethylammonium chloride. In some instances an RT reaction mix comprises gelatin. In some instances an RT reaction mix comprises PEG (PEG1000, PEG2000, PEG4000, PEG6000, PEG8000, or PEG of other length).

Methylome Analysis

Described herein are methods comprising PTA, wherein sites of methylated DNA in single cells are determined using the PTA method. In some instances, these methods further comprise parallel analysis of the transcriptome and/or proteome of the same cell. Methods of detecting methylated genomic bases include selective restriction with methylation-sensitive endonucleases, followed by processing with the PTA method. Sites cut by such enzymes are determined from sequencing, and methylated bases are identified. In another instance, bisulfite treatment of genomic DNA libraries converts unmethylated cytosines to uracil. Libraries are then in some instances amplified with methylation-specific primers which selectively anneal to methylated sequences. Alternatively, non-methylation-specific PCR is conducted, followed by one or more methods to discriminate between bisulfite-reacted bases, including direct pyrosequencing, MS-SnuPE, HRM, COBRA, MS-SSCA, or base-specific cleavage/MALDI-TOF. In some instances, genomic DNA samples are split for parallel analysis of the genome (or an enriched portion thereof) and methylome analysis. In some instances, analysis of the genome and methylome comprises enrichment of genomic fragments (e.g., exome, or other targets) or whole genome sequencing.

Bioinformatics

The data obtained from single-cell analysis methods utilizing PTA described herein may be compiled into a database. Described herein are methods and systems of bioinformatic data integration. Data from the proteome, genome, transcriptome, methylome or other data is in some instances combined/integrated into a database and analyzed. Bioinformatic data integration methods and systems in some instances comprise one or more of protein detection (FACS and/or NGS), mRNA detection, and/or genome variance detection. In some instances, this data is correlated with a disease state or condition. In some instances, data from a plurality of single cells is compiled to describe properties of a larger cell population, such as cells from a specific sample, region, organism, or tissue. In some instances, protein data is acquired from fluorescently labeled antibodies which selectively bind to proteins on a cell. In some instances, a method of protein detection comprises grouping cells based on fluorescent markers and reporting sample location post-sorting. In some instances, a method of protein detection comprises detecting sample barcodes, detecting protein barcodes, comparing to designed sequences, and grouping cells based on barcode and copy number. In some instances, protein data is acquired from barcoded antibodies which selectively bind to proteins on a cell. In some instances, transcriptome data is acquired from sample and RNA specific barcodes. In some instances, a method of mRNA detection comprises detecting sample and RNA specific barcodes, aligning to genome, aligning to RefSeq/Encode, reporting Exon/Intro/Intergenic sequences, analyzing exon-exon junctions, grouping cells based on barcode and expression variance and clustering analysis of variance and top variable genes. In some instances, genomic data is acquired from sample and DNA specific barcodes. In some instances, a method of genome variance detection comprises detecting sample and DNA specific barcodes, aligning to the genome, determine genome recovery and SNV mapping rate, filtering reads on exon-exon junctions, generating variant call file (VCF), and clustering analysis of variance and top variable mutations.

Primary Template-Directed Amplification

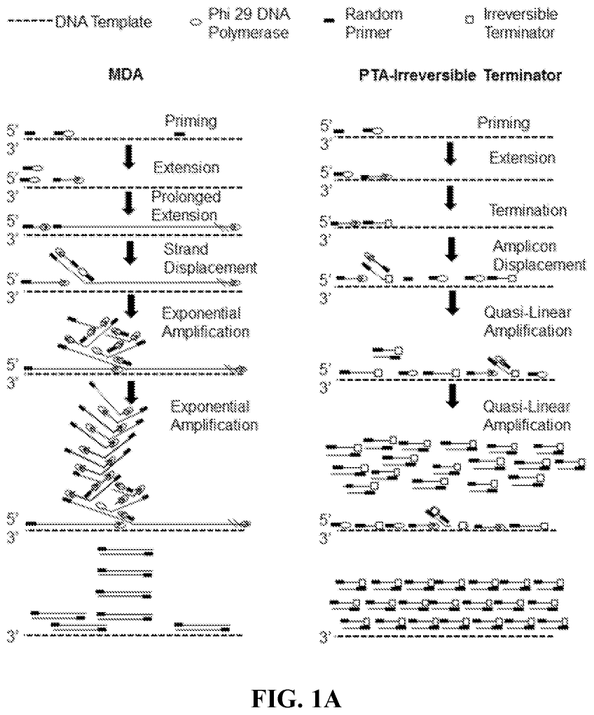

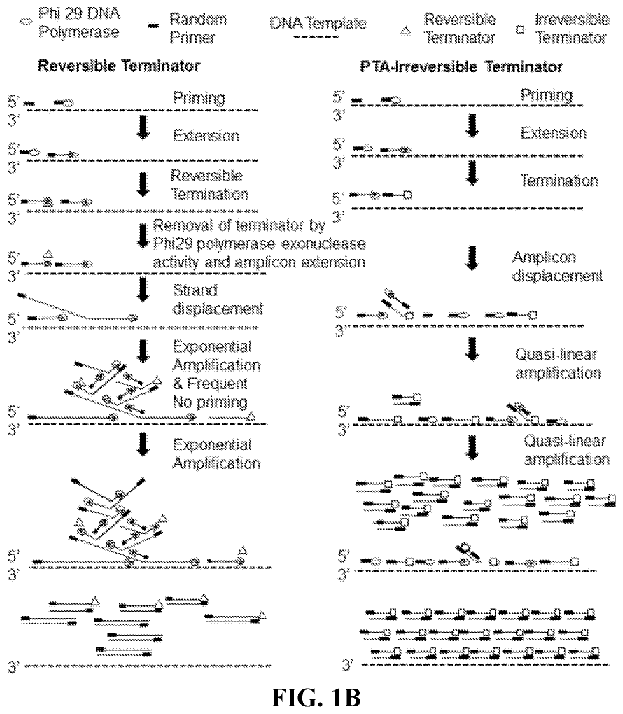

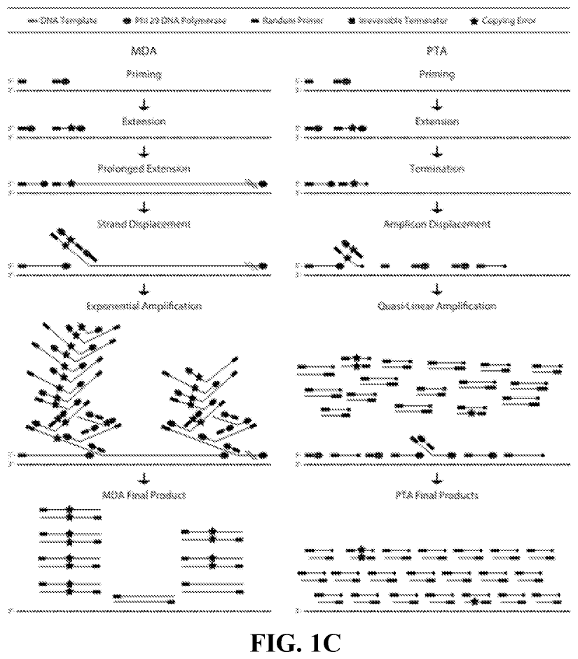

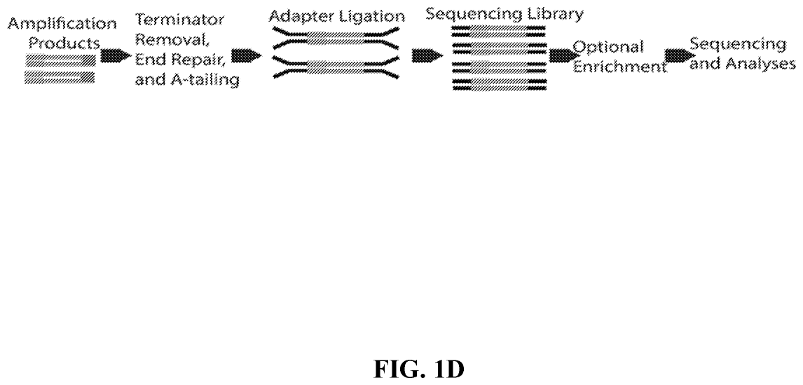

Described herein are nucleic acid amplification methods, such as “Primary Template-Directed Amplification (PTA).” For example, the PTA methods described herein are schematically represented in FIGS. 1A-1D. With the PTA method, amplicons are preferentially generated from the primary template (“direct copies”) using a polymerase (e.g., a strand displacing polymerase). Consequently, errors are propagated at a lower rate from daughter amplicons during subsequent amplifications compared to MDA. The result is an easily executed method that, unlike existing WGA protocols, can amplify low DNA input including the genomes of single cells with high coverage breadth and uniformity in an accurate and reproducible manner. Moreover, the terminated amplification products can undergo direction ligation after removal of the terminators, allowing for the attachment of a cell barcode to the amplification primers so that products from all cells can be pooled after undergoing parallel amplification reactions (FIG. 1D). In some instances, terminator removal is not required prior to amplification and/or adapter ligation.

Described herein are methods employing nucleic acid polymerases with strand displacement activity for amplification. In some instances, such polymerases comprise strand displacement activity and low error rate. In some instances, such polymerases comprise strand displacement activity and proofreading exonuclease activity, such as 3′->5′ proofreading activity. In some instances, nucleic acid polymerases are used in conjunction with other components such as reversible or irreversible terminators, or additional strand displacement factors. In some instances, the polymerase has strand displacement activity, but does not have exonuclease proofreading activity. For example, in some instances such polymerases include bacteriophage phi29 (Φ29) polymerase, which also has very low error rate that is the result of the 3′->5′ proofreading exonuclease activity (see, e.g., U.S. Pat. Nos. 5,198,543 and 5,001,050). In some instances, non-limiting examples of strand displacing nucleic acid polymerases include, e.g., genetically modified phi29 (Φ29) DNA polymerase, Klenow Fragment of DNA polymerase I (Jacobsen et al., Eur. J. Biochem. 45:623-627 (1974)), phage M2 DNA polymerase (Matsumoto et al., Gene 84:247 (1989)), phage phiPRD1 DNA polymerase (Jung et al., Proc. Natl. Acad. Sci. USA 84:8287 (1987); Zhu and Ito, Biochim. Biophys. Acta. 1219:267-276 (1994)), Bst DNA polymerase (e.g., Bst large fragment DNA polymerase (Exo(−) Bst; Aliotta et al., Genet. Anal. (Netherlands) 12:185-195 (1996)), exo(−)Bca DNA polymerase (Walker and Linn, Clinical Chemistry 42:1604-1608 (1996)), Bsu DNA polymerase, Vent_{R} DNA polymerase including Vent_{R} (exo−) DNA polymerase (Kong et al., J. Biol. Chem. 268:1965-1975 (1993)), Deep Vent DNA polymerase including Deep Vent (exo−) DNA polymerase, IsoPol DNA polymerase, DNA polymerase I, Therminator DNA polymerase, T5 DNA polymerase (Chatterjee et al., Gene 97:13-19 (1991)), Sequenase (U.S. Biochemicals), T7 DNA polymerase, T7-Sequenase, T7 gp5 DNA polymerase, PRDI DNA polymerase, T4 DNA polymerase (Kaboord and Benkovic, Curr. Biol. 5:149-157 (1995)). Additional strand displacing nucleic acid polymerases are also compatible with the methods described herein. The ability of a given polymerase to carry out strand displacement replication can be determined, for example, by using the polymerase in a strand displacement replication assay (e.g., as disclosed in U.S. Pat. No. 6,977,148). Such assays in some instances are performed at a temperature suitable for optimal activity for the enzyme being used, for example, 32° C. for phi29 DNA polymerase, from 46° C. to 64° C. for exo(−) Bst DNA polymerase, or from about 60° C. to 70° C. for an enzyme from a hyperthermophylic organism. Another useful assay for selecting a polymerase is the primer-block assay described in Kong et al., J. Biol. Chem. 268:1965-1975 (1993). The assay consists of a primer extension assay using an M13 ssDNA template in the presence or absence of an oligonucleotide that is hybridized upstream of the extending primer to block its progress. Other enzymes capable of displacement the blocking primer in this assay are in some instances useful for the disclosed method. In some instances, polymerases incorporate dNTPs and terminators at approximately equal rates. In some instances, the ratio of rates of incorporation for dNTPs and terminators for a polymerase described herein are about 1:1, about 1.5:1, about 2:1, about 3:1 about 4:1 about 5:1, about 10:1, about 20:1 about 50:1, about 100:1, about 200:1, about 500:1, or about 1000:1. In some instances, the ratio of rates of incorporation for dNTPs and terminators fora polymerase described herein are 1:1 to 1000:1, 2:1 to 500:1, 5:1 to 100:1, 10:1 to 1000:1, 100:1 to 1000:1, 500:1 to 2000:1, 50:1 to 1500:1, or 25:1 to 1000:1.

Described herein are methods of amplification wherein strand displacement can be facilitated through the use of a strand displacement factor, such as, e.g., helicase. Such factors are in some instances used in conjunction with additional amplification components, such as polymerases, terminators, or other component. In some instances, a strand displacement factor is used with a polymerase that does not have strand displacement activity. In some instances, a strand displacement factor is used with a polymerase having strand displacement activity. Without being bound by theory, strand displacement factors may increase the rate that smaller, double stranded amplicons are reprimed. In some instances, any DNA polymerase that can perform strand displacement replication in the presence of a strand displacement factor is suitable for use in the PTA method, even if the DNA polymerase does not perform strand displacement replication in the absence of such a factor. Strand displacement factors useful in strand displacement replication in some instances include (but are not limited to) BMRF1 polymerase accessory subunit (Tsurumi et al., J. Virology 67(12):7648-7653 (1993)), adenovirus DNA-binding protein (Zijderveld and van der Vliet, J. Virology 68(2): 1158-1164 (1994)), herpes simplex viral protein ICP8 (Boehmer and Lehman, J. Virology 67(2):711-715 (1993); Skaliter and Lehman, Proc. Natl. Acad. Sci. USA 91(22):10665-10669 (1994)); single-stranded DNA binding proteins (SSB; Rigler and Romano, J. Biol. Chem. 270:8910-8919 (1995)); phage T4 gene 32 protein (Villemain and Giedroc, Biochemistry 35:14395-14404 (1996);T7 helicase-primase; T7 gp2.5 SSB protein; Tte-UvrD (from Thermoanaerobacter tengcongensis), calf thymus helicase (Siegel et al., J. Biol. Chem. 267:13629-13635 (1992)); bacterial SSB (e.g., E. coli SSB), Replication Protein A (RPA) in eukaryotes, human mitochondrial SSB (mtSSB), and recombinases, (e.g., Recombinase A (RecA) family proteins, T4 UvsX, Sak4 of Phage HK620, Rad51, Dmc1, or Radb). Combinations of factors that facilitate strand displacement and priming are also consistent with the methods described herein. For example, a helicase is used in conjunction with a polymerase. In some instances, the PTA method comprises use of a single-strand DNA binding protein (SSB, T4 gp32, or other single stranded DNA binding protein), a helicase, and a polymerase (e.g., SauDNA polymerase, Bsu polymerase, Bst2.0, GspM, GspM2.0, GspSSD, or other suitable polymerase). In some instances, reverse transcriptases are used in conjunction with the strand displacement factors described herein.

Described herein are amplification methods comprising use of terminator nucleotides, polymerases, and additional factors or conditions. For example, such factors are used in some instances to fragment the nucleic acid template(s) or amplicons during amplification. In some instances, such factors comprise endonucleases. In some instances, factors comprise transposases. In some instances, mechanical shearing is used to fragment nucleic acids during amplification. In some instances, nucleotides are added during amplification that may be fragmented through the addition of additional proteins or conditions. For example, uracil is incorporated into amplicons; treatment with uracil D-glycosylase fragments nucleic acids at uracil-containing positions. Additional systems for selective nucleic acid fragmentation are also in some instances employed, for example an engineered DNA glycosylase that cleaves modified cytosine-pyrene base pairs. (Kwon, et al. Chem Biol. 2003, 10(4), 351).

Described herein are amplification methods comprising use of terminator nucleotides, which terminate nucleic acid replication thus decreasing the size of the amplification products. Such terminators are in some instances used in conjunction with polymerases, strand displacement factors, or other amplification components described herein. In some instances, terminator nucleotides reduce or lower the efficiency of nucleic acid replication. Such terminators in some instances reduce extension rates by at least 99.9%, 99%, 98%, 95%, 90%, 85%, 80%, 75%, 70%, or at least 65%. Such terminators in some instances reduce extension rates by 50%-90%, 60%-80%, 65%-90%, 70%-85%, 60%-90%, 70%-99%, 80%-99%, or 50%-80%. In some instances terminators reduce the average amplicon product length by at least 99.9%, 99%, 98%, 95%, 90%, 85%, 80%, 75%, 70%, or at least 65%. Terminators in some instances reduce the average amplicon length by 50%-90%, 60%-80%, 65%-90%, 70%-85%, 60%-90%, 70%-99%, 80%-99%, or 50%-80%. In some instances, amplicons comprising terminator nucleotides form loops or hairpins which reduce a polymerase's ability to use such amplicons as templates. Use of terminators in some instances slows the rate of amplification at initial amplification sites through the incorporation of terminator nucleotides (e.g., dideoxynucleotides that have been modified to make them exonuclease-resistant to terminate DNA extension), resulting in smaller amplification products. By producing smaller amplification products than the currently used methods (e.g., average length of 50-2000 nucleotides in length for PTA methods as compared to an average product length of >10,000 nucleotides for MDA methods) PTA amplification products in some instances undergo direct ligation of adapters without the need for fragmentation, allowing for efficient incorporation of cell barcodes and unique molecular identifiers (UMI) (see FIGS. 1D, 2B-3E, 5, 6A, and 6B).

Terminator nucleotides are present at various concentrations depending on factors such as polymerase, template, or other factors. For example, the amount of terminator nucleotides in some instances is expressed as a ratio of non-terminator nucleotides to terminator nucleotides in a method described herein. Such concentrations in some instances allow control of amplicon lengths. In some instances, the ratio of non-terminator to terminator nucleotides is about 2:1, 5:1, 7:1, 10:1, 20:1, 50:1, 100:1, 200:1, 500:1, 1000:1, 2000:1, or 5000:1. In some instances the ratio of non-terminator to terminator nucleotides is 2:1-10:1, 5:1-20:1, 10:1-100:1, 20:1-200:1, 50:1-1000:1, 50:1-500:1, 75:1-150:1, or 100:1-500:1. In some instances, at least one of the nucleotides present during amplification using a method described herein is a terminator nucleotide. Each terminator need not be present at approximately the same concentration; in some instances, ratios of each terminator present in a method described herein are optimized for a particular set of reaction conditions, sample type, or polymerase. Without being bound by theory, each terminator may possess a different efficiency for incorporation into the growing polynucleotide chain of an amplicon, in response to pairing with the corresponding nucleotide on the template strand. For example, in some instances a terminator pairing with cytosine is present at about 3%, 5%, 10%, 15%, 20%, 25%, or 50% higher concentration than the average terminator concentration. In some instances a terminator pairing with thymine is present at about 3%, 5%, 10%, 15%, 20%, 25%, or 50% higher concentration than the average terminator concentration. In some instances a terminator pairing with guanine is present at about 3%, 5%, 10%, 15%, 20%, 25%, or 50% higher concentration than the average terminator concentration. In some instances a terminator pairing with adenine is present at about 3%, 5%, 10%, 15%, 20%, 25%, or 50% higher concentration than the average terminator concentration. In some instances a terminator pairing with uracil is present at about 3%, 5%, 10%, 15%, 20%, 25%, or 50% higher concentration than the average terminator concentration. Any nucleotide capable of terminating nucleic acid extension by a nucleic acid polymerase in some instances is used as a terminator nucleotide in the methods described herein. In some instances, a reversible terminator is used to terminate nucleic acid replication. In some instances, a non-reversible terminator is used to terminate nucleic acid replication. In some instances, non-limited examples of terminators include reversible and non-reversible nucleic acids and nucleic acid analogs, such as, e.g., 3′ blocked reversible terminator comprising nucleotides, 3′ unblocked reversible terminator comprising nucleotides, terminators comprising 2′ modifications of deoxynucleotides, terminators comprising modifications to the nitrogenous base of deoxynucleotides, or any combination thereof. In one embodiment, terminator nucleotides are dideoxynucleotides. Other nucleotide modifications that terminate nucleic acid replication and may be suitable for practicing the invention include, without limitation, any modifications of the r group of the 3′ carbon of the deoxyribose such as inverted dideoxynucleotides, 3′ biotinylated nucleotides, 3′ amino nucleotides, 3′-phosphorylated nucleotides, 3′-O-methyl nucleotides, 3′ carbon spacer nucleotides including 3′ C3 spacer nucleotides, 3′ C18 nucleotides, 3′ Hexanediol spacer nucleotides, acyclonucleotides, and combinations thereof. In some instances, terminators are polynucleotides comprising 1, 2, 3, 4, or more bases in length. In some instances, terminators do not comprise a detectable moiety or tag (e.g., mass tag, fluorescent tag, dye, radioactive atom, or other detectable moiety). In some instances, terminators do not comprise a chemical moiety allowing for attachment of a detectable moiety or tag (e.g., “click” azide/alkyne, conjugate addition partner, or other chemical handle for attachment of a tag). In some instances, all terminator nucleotides comprise the same modification that reduces amplification to at region (e.g., the sugar moiety, base moiety, or phosphate moiety) of the nucleotide. In some instances, at least one terminator has a different modification that reduces amplification. In some instances, all terminators have a substantially similar fluorescent excitation or emission wavelengths. In some instances, terminators without modification to the phosphate group are used with polymerases that do not have exonuclease proofreading activity. Terminators, when used with polymerases which have 3′->5′ proofreading exonuclease activity (such as, e.g., phi29) that can remove the terminator nucleotide, are in some instances further modified to make them exonuclease-resistant. For example, dideoxynucleotides are modified with an alpha-thio group that creates a phosphorothioate linkage which makes these nucleotides resistant to the 3′->5′ proofreading exonuclease activity of nucleic acid polymerases. Such modifications in some instances reduce the exonuclease proofreading activity of polymerases by at least 99.5%, 99%, 98%, 95%, 90%, or at least 85%. Non-limiting examples of other terminator nucleotide modifications providing resistance to the 3′->5′ exonuclease activity include in some instances: nucleotides with modification to the alpha group, such as alpha-thio dideoxynucleotides creating a phosphorothioate bond, C3 spacer nucleotides, locked nucleic acids (LNA), inverted nucleic acids, 2′ Fluoro bases, 3′ phosphorylation, 2′-O-Methyl modifications (or other 2′-O-alkyl modification), propyne-modified bases (e.g., deoxycytosine, deoxyuridine), L-DNA nucleotides, L-RNA nucleotides, nucleotides with inverted linkages (e.g., 5′-5′ or 3′-3′), 5′ inverted bases (e.g., 5′ inverted 2′,3′-dideoxy dT), methylphosphonate backbones, and trans nucleic acids. In some instances, nucleotides with modification include base-modified nucleic acids comprising free 3′ OH groups (e.g., 2-nitrobenzyl alkylated HOMedU triphosphates, bases comprising modification with large chemical groups, such as solid supports or other large moiety). In some instances, a polymerase with strand displacement activity but without 3′->5′exonuclease proofreading activity is used with terminator nucleotides with or without modifications to make them exonuclease resistant. Such nucleic acid polymerases include, without limitation, Bst DNA polymerase, Bsu DNA polymerase, Deep Vent (exo−) DNA polymerase, Klenow Fragment (exo−) DNA polymerase, Therminator DNA polymerase, and VentR (exo−).

Primers and Amplicon Libraries

Described herein are amplicon libraries resulting from amplification of at least one target nucleic acid molecule. Such libraries are in some instances generated using the methods described herein, such as those using terminators. Such methods comprise use of strand displacement polymerases or factors, terminator nucleotides (reversible or irreversible), or other features and embodiments described herein. In some instances, amplicon libraries generated by use of terminators described herein are further amplified in a subsequent amplification reaction (e.g., PCR). In some instances, subsequent amplification reactions do not comprise terminators. In some instances, amplicon libraries comprise polynucleotides, wherein at least 50%, 60%, 70%, 80%, 90%, 95%, or at least 98% of the polynucleotides comprise at least one terminator nucleotide. In some instances, the amplicon library comprises the target nucleic acid molecule from which the amplicon library was derived. The amplicon library comprises a plurality of polynucleotides, wherein at least some of the polynucleotides are direct copies (e.g., replicated directly from a target nucleic acid molecule, such as genomic DNA, RNA, or other target nucleic acid). For example, at least 5%, 10%, 20%, 30%, 40%, 50%, 60%, 70%, 80%, 90%, 95% or more than 95% of the amplicon polynucleotides are direct copies of the at least one target nucleic acid molecule. In some instances, at least 5% of the amplicon polynucleotides are direct copies of the at least one target nucleic acid molecule. In some instances, at least 10% of the amplicon polynucleotides are direct copies of the at least one target nucleic acid molecule. In some instances, at least 15% of the amplicon polynucleotides are direct copies of the at least one target nucleic acid molecule. In some instances, at least 20% of the amplicon polynucleotides are direct copies of the at least one target nucleic acid molecule. In some instances, at least 50% of the amplicon polynucleotides are direct copies of the at least one target nucleic acid molecule. In some instances, 3%-5%, 3-10%, 5%-10%, 10%-20%, 20%-30%, 30%-40%, 5%-30%, 10%-50%, or 15%-75% of the amplicon polynucleotides are direct copies of the at least one target nucleic acid molecule. In some instances, at least some of the polynucleotides are direct copies of the target nucleic acid molecule, or daughter (a first copy of the target nucleic acid) progeny. For example, at least 5%, 10%, 20%, 30%, 40%, 50%, 60%, 70%, 80%, 90%, 95% or more than 95% of the amplicon polynucleotides are direct copies of the at least one target nucleic acid molecule or daughter progeny. In some instances, at least 5% of the amplicon polynucleotides are direct copies of the at least one target nucleic acid molecule or daughter progeny. In some instances, at least 10% of the amplicon polynucleotides are direct copies of the at least one target nucleic acid molecule or daughter progeny. In some instances, at least 20% of the amplicon polynucleotides are direct copies of the at least one target nucleic acid molecule or daughter progeny. In some instances, at least 30% of the amplicon polynucleotides are direct copies of the at least one target nucleic acid molecule or daughter progeny. In some instances, 3%-5%, 3%-10%, 5%-10%, 10%-20%, 20%-30%, 30%-40%, 5%-30%, 10%-50%, or 15%-75% of the amplicon polynucleotides are direct copies of the at least one target nucleic acid molecule or daughter progeny. In some instances, direct copies of the target nucleic acid are 50-2500, 75-2000, 50-2000, 25-1000, 50-1000, 500-2000, or 50-2000 bases in length. In some instances, daughter progeny are 1000-5000, 2000-5000, 1000-10,000, 2000-5000, 1500-5000, 3000-7000, or 2000-7000 bases in length. In some instances, the average length of PTA amplification products is 25-3000 nucleotides in length, 50-2500, 75-2000, 50-2000, 25-1000, 50-1000, 500-2000, or 50-2000 bases in length. In some instance, amplicons generated from PTA are no more than 5000, 4000, 3000, 2000, 1700, 1500, 1200, 1000, 700, 500, or no more than 300 bases in length. In some instance, amplicons generated from PTA are 1000-5000, 1000-3000, 200-2000, 200-4000, 500-2000, 750-2500, or 1000-2000 bases in length. Amplicon libraries generated using the methods described herein in some instances comprise at least 1000, 2000, 5000, 10,000, 100,000, 200,000, 500,000 or more than 500,000 amplicons comprising unique sequences. In some instances, the library comprises at least 100, 200, 300, 400, 500, 600, 700, 800, 900, 1000, 1100, 1200, 1300, 1400, 1500, 2000, 2500, 3000, or at least 3500 amplicons. In some instances, at least 5%, 10%, 15%, 20%, 25%, 30% or more than 30% of amplicon polynucleotides having a length of less than 1000 bases are direct copies of the at least one target nucleic acid molecule. In some instances, at least 5%, 10%, 15%, 20%, 25%, 30% or more than 30% of amplicon polynucleotides having a length of no more than 2000 bases are direct copies of the at least one target nucleic acid molecule. In some instances, at least 5%, 10%, 15%, 20%, 25%, 30% or more than 30% of amplicon polynucleotides having a length of 3000-5000 bases are direct copies of the at least one target nucleic acid molecule. In some instances, the ratio of direct copy amplicons to target nucleic acid molecules is at least 10:1, 100:1, 1000:1, 10,000:1, 100,000:1, 1,000,000:1, 10,000,000:1, or more than 10,000,000:1. In some instances, the ratio of direct copy amplicons to target nucleic acid molecules is at least 10:1, 100:1, 1000:1, 10,000:1, 100,000:1, 1,000,000:1, 10,000,000:1, or more than 10,000,000:1, wherein the direct copy amplicons are no more than 700-1200 bases in length. In some instances, the ratio of direct copy amplicons and daughter amplicons to target nucleic acid molecules is at least 10:1, 100:1, 1000:1, 10,000:1, 100,000:1, 1,000,000:1, 10,000,000:1, or more than 10,000,000:1. In some instances, the ratio of direct copy amplicons and daughter amplicons to target nucleic acid molecules is at least 10:1, 100:1, 1000:1, 10,000:1, 100,000:1, 1,000,000:1, 10,000,000:1, or more than 10,000,000:1, wherein the direct copy amplicons are 700-1200 bases in length, and the daughter amplicons are 2500-6000 bases in length. In some instances, the library comprises about 50-10,000, about 50-5,000, about 50-2500, about 50-1000, about 150-2000, about 250-3000, about 50-2000, about 500-2000, or about 500-1500 amplicons which are direct copies of the target nucleic acid molecule. In some instances, the library comprises about 50-10,000, about 50-5,000, about 50-2500, about 50-1000, about 150-2000, about 250-3000, about 50-2000, about 500-2000, or about 500-1500 amplicons which are direct copies of the target nucleic acid molecule or daughter amplicons. The number of direct copies may be controlled in some instances by the number of PCR amplification cycles. In some instances, no more than 30, 25, 20, 15, 13, 11, 10, 9, 8, 7, 6, 5, 4, or 3 are used to generate copies of the target nucleic acid molecule. In some instances, about 30, 25, 20, 15, 13, 11, 10, 9, 8, 7, 6, 5, 4, or about 3 PCR cycles are used to generate copies of the target nucleic acid molecule. In some instances, 3, 4, 5, 6, 7, or 8 PCR cycles are used to generate copies of the target nucleic acid molecule. In some instances, 2-4, 2-5, 2-7, 2-8, 2-10, 2-15, 3-5, 3-10, 3-15, 4-10, 4-15, 5-10 or 5-15 PCR cycles are used to generate copies of the target nucleic acid molecule. Amplicon libraries generated using the methods described herein are in some instances subjected to additional steps, such as adapter ligation and further PCR amplification. In some instances, such additional steps precede a sequencing step. In some instances, no more than 30, 25, 20, 15, 13, 11, 10, 9, 8, 7, 6, 5, 4, or 3 cycles are used to generate copies of the target nucleic acid molecule. In some instances, about 30, 25, 20, 15, 13, 11, 10, 9, 8, 7, 6, 5, 4, or about 3 cycles are used to generate copies of the target nucleic acid molecule. In some instances, 3, 4, 5, 6, 7, or 8 cycles are used to generate copies of the target nucleic acid molecule. In some instances, 2-4, 2-5, 2-7, 2-8, 2-10, 2-15, 3-5, 3-10, 3-15, 4-10, 4-15, 5-10 or 5-15 cycles are used to generate copies of the target nucleic acid molecule. Amplicon libraries generated using the methods described herein are in some instances subjected to additional steps, such as adapter ligation and further amplification. In some instances, such additional steps precede a sequencing step. In some instances, the cycles are PCR cycles. In some instances, the cycles represent annealing, extension, and denaturation. In some instances, the cycles represent annealing, extension, and denaturation which occur under isothermal or essentially isothermal conditions.

Amplicon libraries of polynucleotides generated from the PTA methods and compositions (terminators, polymerases, etc.) described herein in some instances have increased uniformity. Uniformity, in some instances, is described using a Lorenz curve or other such method. Such increases in some instances lead to lower sequencing reads needed for the desired coverage of a target nucleic acid molecule (e.g., genomic DNA, RNA, or other target nucleic acid molecule). For example, no more than 50% of a cumulative fraction of polynucleotides comprises sequences of at least 80% of a cumulative fraction of sequences of the target nucleic acid molecule. In some instances, no more than 50% of a cumulative fraction of polynucleotides comprises sequences of at least 60% of a cumulative fraction of sequences of the target nucleic acid molecule. In some instances, no more than 50% of a cumulative fraction of polynucleotides comprises sequences of at least 70% of a cumulative fraction of sequences of the target nucleic acid molecule. In some instances, no more than 50% of a cumulative fraction of polynucleotides comprises sequences of at least 90% of a cumulative fraction of sequences of the target nucleic acid molecule. In some instances, uniformity is described using a Gini index (wherein an index of 0 represents perfect equality of the library and an index of 1 represents perfect inequality). In some instances, amplicon libraries described herein have a Gini index of no more than 0.55, 0.50, 0.45, 0.40, or 0.30. In some instances, amplicon libraries described herein have a Gini index of no more than 0.50. In some instances, amplicon libraries described herein have a Gini index of no more than 0.40. Such uniformity metrics in some instances are dependent on the number of reads obtained. For example no more than 100 million, 200 million, 300 million, 400 million, or no more than 500 million reads are obtained. In some instances, the read length is about 50,75, 100, 125, 150, 175, 200, 225, or about 250 bases in length. In some instances, uniformity metrics are dependent on the depth of coverage of a target nucleic acid. For example, the average depth of coverage is about 10×, 15×, 20×, 25×, or about 30×. In some instances, the average depth of coverage is 10-30×, 20-50×, 5-40×, 20-60×, 5-20×, or 10-20×. In some instances, amplicon libraries described herein have a Gini index of no more than 0.55, wherein about 300 million reads was obtained. In some instances, amplicon libraries described herein have a Gini index of no more than 0.50, wherein about 300 million reads was obtained. In some instances, amplicon libraries described herein have a Gini index of no more than 0.45, wherein about 300 million reads was obtained. In some instances, amplicon libraries described herein have a Gini index of no more than 0.55, wherein no more than 300 million reads was obtained. In some instances, amplicon libraries described herein have a Gini index of no more than 0.50, wherein no more than 300 million reads was obtained. In some instances, amplicon libraries described herein have a Gini index of no more than 0.45, wherein no more than 300 million reads was obtained. In some instances, amplicon libraries described herein have a Gini index of no more than 0.55, wherein the average depth of sequencing coverage is about 15×. In some instances, amplicon libraries described herein have a Gini index of no more than 0.50, wherein the average depth of sequencing coverage is about 15×. In some instances, amplicon libraries described herein have a Gini index of no more than 0.45, wherein the average depth of sequencing coverage is about 15×. In some instances, amplicon libraries described herein have a Gini index of no more than 0.55, wherein the average depth of sequencing coverage is at least 15×. In some instances, amplicon libraries described herein have a Gini index of no more than 0.50, wherein the average depth of sequencing coverage is at least 15×. In some instances, amplicon libraries described herein have a Gini index of no more than 0.45, wherein the average depth of sequencing coverage is at least 15×. In some instances, amplicon libraries described herein have a Gini index of no more than 0.55, wherein the average depth of sequencing coverage is no more than 15×. In some instances, amplicon libraries described herein have a Gini index of no more than 0.50, wherein the average depth of sequencing coverage is no more than 15×. In some instances, amplicon libraries described herein have a Gini index of no more than 0.45, wherein the average depth of sequencing coverage is no more than 15×. Uniform amplicon libraries generated using the methods described herein are in some instances subjected to additional steps, such as adapter ligation and further PCR amplification. In some instances, such additional steps precede a sequencing step.

Primers comprise nucleic acids used for priming the amplification reactions described herein. Such primers in some instances include, without limitation, random deoxynucleotides of any length with or without modifications to make them exonuclease resistant, random ribonucleotides of any length with or without modifications to make them exonuclease resistant, modified nucleic acids such as locked nucleic acids, DNA or RNA primers that are targeted to a specific genomic region, and reactions that are primed with enzymes such as primase. In the case of whole genome PTA, it is preferred that a set of primers having random or partially random nucleotide sequences be used. In a nucleic acid sample of significant complexity, specific nucleic acid sequences present in the sample need not be known and the primers need not be designed to be complementary to any particular sequence. Rather, the complexity of the nucleic acid sample results in a large number of different hybridization target sequences in the sample, which will be complementary to various primers of random or partially random sequence. The complementary portion of primers for use in PTA are in some instances fully randomized, comprise only a portion that is randomized, or be otherwise selectively randomized. The number of random base positions in the complementary portion of primers in some instances, for example, is from 20% to 100% of the total number of nucleotides in the complementary portion of the primers. In some instances, the number of random base positions in the complementary portion of primers is 10% to 90%, 15-95%, 20%-100%, 30%-100%, 50%-100%, 75-100% or 90-95% of the total number of nucleotides in the complementary portion of the primers. In some instances, the number of random base positions in the complementary portion of primers is at least 10%, 20%, 30%, 40%, 50%, 60%, 70%, 80%, or at least 90% of the total number of nucleotides in the complementary portion of the primers. Sets of primers having random or partially random sequences are in some instances synthesized using standard techniques by allowing the addition of any nucleotide at each position to be randomized. In some instances, sets of primers are composed of primers of similar length and/or hybridization characteristics. In some instances, the term “random primer” refers to a primer which can exhibit four-fold degeneracy at each position. In some instances, the term “random primer” refers to a primer which can exhibit three-fold degeneracy at each position. Random primers used in the methods described herein in some instances comprise a random sequence that is 3, 4, 5, 6, 7, 8, 10, 11, 12, 13, 14, 15, 16, 17, 18, 19, 20, or more bases in length. In some instances, primers comprise random sequences that are 3-20, 5-15, 5-20, 6-12, or 4-10 bases in length. Primers may also comprise non-extendable elements that limit subsequent amplification of amplicons generated thereof. For example, primers with non-extendable elements in some instances comprise terminators. In some instances, primers comprise terminator nucleotides, such as 1, 2, 3, 4, 5, 10, or more than 10 terminator nucleotides. Primers need not be limited to components which are added externally to an amplification reaction. In some instances, primers are generated in-situ through the addition of nucleotides and proteins which promote priming. For example, primase-like enzymes in combination with nucleotides is in some instances used to generate random primers for the methods described herein. Primase-like enzymes in some instances are members of the DnaG or AEP enzyme superfamily. In some instances, a primase-like enzyme is TthPrimPol. In some instances, a primase-like enzyme is T7 gp4 helicase-primase. Such primases are in some instances used with the polymerases or strand displacement factors described herein. In some instances, primases initiate priming with deoxyribonucleotides. In some instances, primases initiate priming with ribonucleotides.

The PTA amplification can be followed by selection for a specific subset of amplicons. Such selections are in some instances dependent on size, affinity, activity, hybridization to probes, or other known selection factor in the art. In some instances, selections precede or follow additional steps described herein, such as adapter ligation and/or library amplification. In some instances, selections are based on size (length) of the amplicons. In some instances, smaller amplicons are selected that are less likely to have undergone exponential amplification, which enriches for products that were derived from the primary template while further converting the amplification from an exponential into a quasi-linear amplification process (FIG. 1A). In some instances, amplicons comprising 50-2000, 25-5000, 40-3000, 50-1000, 200-1000, 300-1000, 400-1000, 400-600, 600-2000, or 800-1000 bases in length are selected. Size selection in some instances occurs with the use of protocols, e.g., utilizing solid-phase reversible immobilization (SPRI) on carboxylated paramagnetic beads to enrich for nucleic acid fragments of specific sizes, or other protocol known by those skilled in the art. Optionally or in combination, selection occurs through preferential amplification of smaller fragments during PCR while preparing sequencing libraries, as well as a result of the preferential formation of clusters from smaller sequencing library fragments during Illumina sequencing. Other strategies to select for smaller fragments are also consistent with the methods described herein and include, without limitation, isolating nucleic acid fragments of specific sizes after gel electrophoresis, the use of silica columns that bind nucleic acid fragments of specific sizes, and the use of other PCR strategies that more strongly enrich for smaller fragments. Any number of library preparation protocols may be used with the PTA methods described herein. Amplicons generated by PTA are in some instances ligated to adapters (optionally with removal of terminator nucleotides). In some instances, amplicons generated by PTA comprise regions of homology generated from transposase-based fragmentation which are used as priming sites.

The non-complementary portion of a primer used in PTA can include sequences which can be used to further manipulate and/or analyze amplified sequences. An example of such a sequence is a “detection tag”. Detection tags have sequences complementary to detection probes and are detected using their cognate detection probes. There may be one, two, three, four, or more than four detection tags on a primer. There is no fundamental limit to the number of detection tags that can be present on a primer except the size of the primer. In some instances, there is a single detection tag on a primer. In some instances, there are two detection tags on a primer. When there are multiple detection tags, they may have the same sequence or they may have different sequences, with each different sequence complementary to a different detection probe. In some instances, multiple detection tags have the same sequence. In some instances, multiple detection tags have a different sequence.

Another example of a sequence that can be included in the non-complementary portion of a primer is an “address tag” that can encode other details of the amplicons, such as the location in a tissue section. In some instances, a cell barcode comprises an address tag. An address tag has a sequence complementary to an address probe. Address tags become incorporated at the ends of amplified strands. If present, there may be one, or more than one, address tag on a primer. There is no fundamental limit to the number of address tags that can be present on a primer except the size of the primer. When there are multiple address tags, they may have the same sequence or they may have different sequences, with each different sequence complementary to a different address probe. The address tag portion can be any length that supports specific and stable hybridization between the address tag and the address probe. In some instances, nucleic acids from more than one source can incorporate a variable tag sequence. This tag sequence can be up to 100 nucleotides in length, preferably 1 to 10 nucleotides in length, most preferably 4, 5 or 6 nucleotides in length and comprises combinations of nucleotides. In some instances, a tag sequence is 1-20, 2-15, 3-13, 4-12, 5-12, or 1-10 nucleotides in length For example, if six base-pairs are chosen to form the tag and a permutation of four different nucleotides is used, then a total of 4096 nucleic acid anchors (e.g. hairpins), each with a unique 6 base tag can be made.

Primers described herein may be present in solution or immobilized on a solid support. In some instances, primers bearing sample barcodes and/or UMI sequences can be immobilized on a solid support. The solid support can be, for example, one or more beads. In some instances, individual cells are contacted with one or more beads having a unique set of sample barcodes and/or UMI sequences in order to identify the individual cell. In some instances, lysates from individual cells are contacted with one or more beads having a unique set of sample barcodes and/or UMI sequences in order to identify the individual cell lysates. In some instances, purified nucleic acid from individual cells are contacted with one or more beads having a unique set of sample barcodes and/or UMI sequences in order to identify the purified nucleic acid from the individual cell. The beads can be manipulated in any suitable manner as is known in the art, for example, using droplet actuators as described herein. The beads may be any suitable size, including for example, microbeads, microparticles, nanobeads and nanoparticles. In some embodiments, beads are magnetically responsive; in other embodiments beads are not significantly magnetically responsive. Non-limiting examples of suitable beads include flow cytometry microbeads, polystyrene microparticles and nanoparticles, functionalized polystyrene microparticles and nanoparticles, coated polystyrene microparticles and nanoparticles, silica microbeads, fluorescent microspheres and nanospheres, functionalized fluorescent microspheres and nanospheres, coated fluorescent microspheres and nanospheres, color dyed microparticles and nanoparticles, magnetic microparticles and nanoparticles, superparamagnetic microparticles and nanoparticles (e.g., DYNABEADS® available from Invitrogen Group, Carlsbad, Calif.), fluorescent microparticles and nanoparticles, coated magnetic microparticles and nanoparticles, ferromagnetic microparticles and nanoparticles, coated ferromagnetic microparticles and nanoparticles, and those described in U.S. Pat. Appl. Pub. No. US20050260686, US20030132538, US20050118574, 20050277197, 20060159962. Beads may be pre-coupled with an antibody, protein or antigen, DNA/RNA probe or any other molecule with an affinity for a desired target. In some embodiments, primers bearing sample barcodes and/or UMI sequences can be in solution. In certain embodiments, a plurality of droplets can be presented, wherein each droplet in the plurality bears a sample barcode which is unique to a droplet and the UMI which is unique to a molecule such that the UMI are repeated many times within a collection of droplets. In some embodiments, individual cells are contacted with a droplet having a unique set of sample barcodes and/or UMI sequences in order to identify the individual cell. In some embodiments, lysates from individual cells are contacted with a droplet having a unique set of sample barcodes and/or UMI sequences in order to identify the individual cell lysates. In some embodiments, purified nucleic acid from individual cells are contacted with a droplet having a unique set of sample barcodes and/or UMI sequences in order to identify the purified nucleic acid from the individual cell. Various microfluidics platforms may be used for analysis of single cells. Cells in some instances are manipulated through hydrodynamics (droplet microfluidics, inertial microfluidics, vortexing, microvalves, microstructures (e.g., microwells, microtraps)), electrical methods (dielectrophoresis (DEP), electroosmosis), optical methods (optical tweezers, optically induced dielectrophoresis (ODEP), opto-thermocapillary), acoustic methods, or magnetic methods. In some instances, the microfluidics platform comprises microwells. In some instances, the microfluidics platform comprises a PDMS (Polydimethylsiloxane)-based device. Non-limited examples of single cell analysis platforms compatible with the methods described herein are: ddSEQ Single-Cell Isolator, (Bio-Rad, Hercules, Calif., USA, and Illumina, San Diego, Calif., USA)); Chromium (10× Genomics, Pleasanton, Calif., USA)); Rhapsody Single-Cell Analysis System (BD, Franklin Lakes, N.J., USA); Tapestri Platform (MissionBio, San Francisco, Calif., USA)), Nadia Innovate (Dolomite Bio, Royston, UK); C1 and Polaris (Fluidigm, South San Francisco, Calif.f, USA); ICELL8 Single-Cell System (Takara); MSND (Wafergen); Puncher platform (Vycap); CellRaft AIR System (CellMicrosystems); DEPArray NxT and DEPArray System (Menarini Silicon Biosystems); AVISO CellCelector (ALS); and InDrop System (1CellBio).

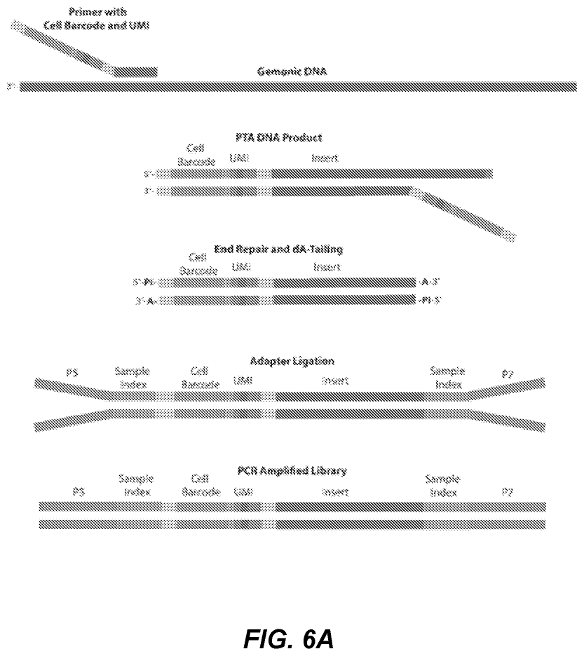

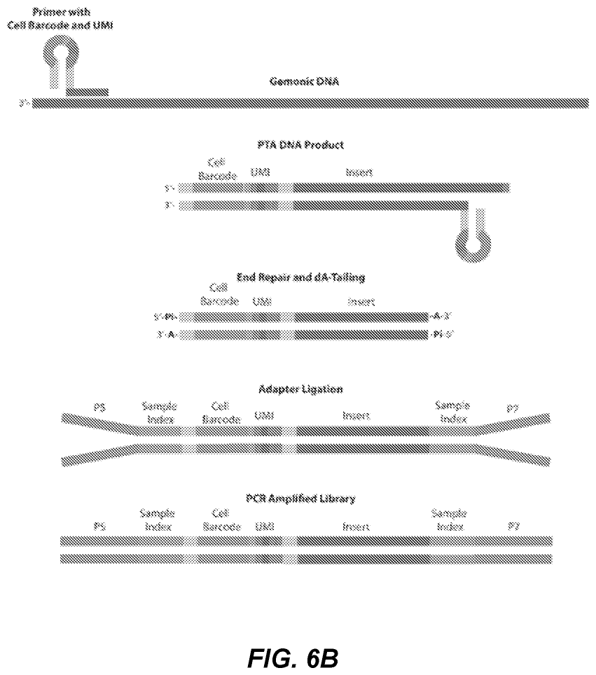

PTA primers may comprise a sequence-specific or random primer, an address tag, a cell barcode and/or a unique molecular identifier (UMI) (see, e.g., FIGS. 6A (linear primer) and 6B (hairpin primer)). In some instances, a primer comprises a sequence-specific primer. In some instances, a primer comprises a random primer. In some instances, a primer comprises a cell barcode. In some instances, a primer comprises a sample barcode. In some instances, a primer comprises a unique molecular identifier. In some instances, primers comprise two or more cell barcodes. Such barcodes in some instances identify a unique sample source, or unique workflow. Such barcodes or UMIs are in some instances 5, 6, 7, 8, 9, 10, 11, 12, 15, 20, 25, 30, or more than 30 bases in length. Primers in some instances comprise at least 1000, 10,000, 50,000, 100,000, 250,000, 500,000, 10⁶, 10⁷, 10⁸, 10⁹, or at least 10¹⁰ unique barcodes or UMIs. In some instances primers comprise at least 8, 16, 96, or 384 unique barcodes or UMIs. In some instances a standard adapter is then ligated onto the amplification products prior to sequencing; after sequencing, reads are first assigned to a specific cell based on the cell barcode. Suitable adapters that may be utilized with the PTA method include, e.g., xGen® Dual Index UMI adapters available from Integrated DNA Technologies (IDT). Reads from each cell is then grouped using the UMI, and reads with the same UMI may be collapsed into a consensus read. The use of a cell barcode allows all cells to be pooled prior to library preparation, as they can later be identified by the cell barcode. The use of the UMI to form a consensus read in some instances corrects for PCR bias, improving the copy number variation (CNV) detection. In addition, sequencing errors may be corrected by requiring that a fixed percentage of reads from the same molecule have the same base change detected at each position. This approach has been utilized to improve CNV detection and correct sequencing errors in bulk samples. In some instances, UMIs are used with the methods described herein, for example, U.S. Pat. No. 8,835,358 discloses the principle of digital counting after attaching a random amplifiable barcode. Schmitt. et al and Fan et al. disclose similar methods of correcting sequencing errors.

The methods described herein may further comprise additional steps, including steps performed on the sample or template. Such samples or templates in some instance are subjected to one or more steps prior to PTA. In some instances, samples comprising cells are subjected to a pre-treatment step. For example, cells undergo lysis and proteolysis to increase chromatin accessibility using a combination of freeze-thawing, Triton X-100, Tween 20, and Proteinase K. Other lysis strategies are also be suitable for practicing the methods described herein. Such strategies include, without limitation, lysis using other combinations of detergent and/or lysozyme and/or protease treatment and/or physical disruption of cells such as sonication and/or alkaline lysis and/or hypotonic lysis. In some instances, cells are lysed with mechanical (e.g., high pressure homogenizer, bead milling) or non-mechanical (physical, chemical, or biological). In some instances, physical lysis methods comprise heating, osmotic shock, and/or cavitation. In some instances, chemical lysis comprises alkali and/or detergents. In some instances, biological lysis comprises use of enzymes. Combinations of lysis methods are also compatible with the methods described herein. Non-limited examples of lysis enzymes include recombinant lysozyme, serine proteases, and bacterial lysins. In some instances, lysis with enzymes comprises use of lysozyme, lysostaphin, zymolase, cellulose, protease or glycanase. In some instances, the primary template or target molecule(s) is subjected to a pre-treatment step. In some instances, the primary template (or target) is denatured using sodium hydroxide, followed by neutralization of the solution. Other denaturing strategies may also be suitable for practicing the methods described herein. Such strategies may include, without limitation, combinations of alkaline lysis with other basic solutions, increasing the temperature of the sample and/or altering the salt concentration in the sample, addition of additives such as solvents or oils, other modification, or any combination thereof. In some instances, additional steps include sorting, filtering, or isolating samples, templates, or amplicons by size. For example, after amplification with the methods described herein, amplicon libraries are enriched for amplicons having a desired length. In some instances, amplicon libraries are enriched for amplicons having a length of 50-2000, 25-1000, 50-1000, 75-2000, 100-3000, 150-500, 75-250, 170-500, 100-500, or 75-2000 bases. In some instances, amplicon libraries are enriched for amplicons having a length no more than 75, 100, 150, 200, 500, 750, 1000, 2000, 5000, or no more than 10,000 bases. In some instances, amplicon libraries are enriched for amplicons having a length of at least 25, 50, 75, 100, 150, 200, 500, 750, 1000, or at least 2000 bases.

Methods and compositions described herein may comprise buffers or other formulations. Such buffers in some instances comprise surfactants/detergent or denaturing agents (Tween-20, DMSO, DMF, pegylated polymers comprising a hydrophobic group, or other surfactant), salts (potassium or sodium phosphate (monobasic or dibasic), sodium chloride, potassium chloride, TrisHCl, magnesium chloride or sulfate, Ammonium salts such as phosphate, nitrate, or sulfate, EDTA), reducing agents (DTT, THP, DTE, beta-mercaptoethanol, TCEP, or other reducing agent) or other components (glycerol, hydrophilic polymers such as PEG). In some instances, buffers are used in conjunction with components such as polymerases, strand displacement factors, terminators, or other reaction component described herein. Buffers may comprise one or more crowding agents. In some instances, crowding reagents include polymers. In some instances, crowding reagents comprise polymers such as polyols. In some instances, crowding reagents comprise polyethylene glycol polymers (PEG). In some instances, crowding reagents comprise polysaccharides. Without limitation, examples of crowding reagents include ficoll (e.g., ficoll PM 400, ficoll PM 70, or other molecular weight ficoll), PEG (e.g., PEG1000, PEG 2000, PEG4000, PEG6000, PEG8000, or other molecular weight PEG), dextran (dextran 6, dextran 10, dextran 40, dextran 70, dextran 6000, dextran 138k, or other molecular weight dextran).

The nucleic acid molecules amplified according to the methods described herein may be sequenced and analyzed using methods known to those of skill in the art. Non-limiting examples of the sequencing methods which in some instances are used include, e.g., sequencing by hybridization (SBH), sequencing by ligation (SBL) (Shendure et al. (2005) Science 309:1728), quantitative incremental fluorescent nucleotide addition sequencing (QIFNAS), stepwise ligation and cleavage, fluorescence resonance energy transfer (FRET), molecular beacons, TaqMan reporter probe digestion, pyrosequencing, fluorescent in situ sequencing (FISSEQ), FISSEQ beads (U.S. Pat. No. 7,425,431), wobble sequencing (Int. Pat. Appl. Pub. No. WO2006/073504), multiplex sequencing (U.S. Pat. Appl. Pub. No. US2008/0269068; Porreca et al., 2007, Nat. Methods 4:931), polymerized colony (POLONY) sequencing (U.S. Pat. Nos. 6,432,360, 6,485,944 and 6,511,803, and Int. Pat. Appl. Pub. No. WO2005/082098), nanogrid rolling circle sequencing (ROLONY) (U.S. Pat. No. 9,624,538), allele-specific oligo ligation assays (e.g., oligo ligation assay (OLA), single template molecule OLA using a ligated linear probe and a rolling circle amplification (RCA) readout, ligated padlock probes, and/or single template molecule OLA using a ligated circular padlock probe and a rolling circle amplification (RCA) readout), high-throughput sequencing methods such as, e.g., methods using Roche 454, Illumina Solexa, AB-SOLiD, Helicos, Polonator platforms and the like, and light-based sequencing technologies (Landegren et al. (1998) Genome Res. 8:769-76; Kwok (2000) Pharmacogenomics 1:95-100; and Shi (2001) Clin. Chem.47:164-172). In some instances, the amplified nucleic acid molecules are shotgun sequenced.

Described herein are methods generating amplicon libraries from samples comprising short nucleic acid using the PTA methods described herein. In some instances, PTA leads to improved fidelity and uniformity of amplification of shorter nucleic acids. In some instances, nucleic acids are no more than 2000 bases in length. In some instances, nucleic acids are no more than 1000 bases in length. In some instances, nucleic acids are no more than 500 bases in length. In some instances, nucleic acids are no more than 200, 400, 750, 1000, 2000 or 5000 bases in length. In some instances, samples comprising short nucleic acid fragments include but at not limited to ancient DNA (hundreds, thousands, millions, or even billions of years old), FFPE (Formalin-Fixed Paraffin-Embedded) samples, cell-free DNA, or other sample comprising short nucleic acids.

### Kits

Described herein are kits facilitating the practice of the PTA method. Various combinations of the components set forth above in regard to exemplary reaction mixtures and reaction methods can be provided in a kit form. A kit may include individual components that are separated from each other, for example, being carried in separate vessels or packages. A kit in some instances includes one or more sub-combinations of the components set forth herein, the one or more sub-combinations being separated from other components of the kit. The sub-combinations in some instances are combinable to create a reaction mixture set forth herein (or combined to perform a reaction set forth herein). In particular embodiments, a sub-combination of components that is present in an individual vessel or package is insufficient to perform a reaction set forth herein. However, the kit as a whole in some instances includes a collection of vessels or packages the contents of which can be combined to perform a reaction set forth herein.

A kit can include a suitable packaging material to house the contents of the kit. The packaging material in some instances is constructed by well-known methods, preferably to provide a sterile, contaminant-free environment. The packaging materials employed herein include, for example, those customarily utilized in commercial kits sold for use with nucleic acid sequencing systems. Exemplary packaging materials include, without limitation, glass, plastic, paper, foil, and the like, capable of holding within fixed limits a component set forth herein. The packaging material can include a label which indicates a particular use for the components. The use for the kit that is indicated by the label in some in instances is one or more of the methods set forth herein as appropriate for the particular combination of components present in the kit. For example, a label in some instances indicates that the kit is useful for a method of detecting mutations in a nucleic acid sample using the PTA method. Instructions for use of the packaged reagents or components can also be included in a kit. The instructions will typically include a tangible expression describing reaction parameters, such as the relative amounts of kit components and sample to be admixed, maintenance time periods for reagent/sample admixtures, temperature, buffer conditions, and the like. It will be understood that not all components necessary for a particular reaction need be present in a particular kit. Rather one or more additional components in some instances are provided from other sources. The instructions provided with a kit in some instances identify the additional component(s) that are to be provided and where they can be obtained. In one embodiment, a kit provides at least one amplification primer; at least one nucleic acid polymerase; a mixture of at least two nucleotides, wherein the mixture of nucleotides comprises at least one terminator nucleotide which terminates nucleic acid replication by the polymerase; and instructions for use of the kit. In some instances, the kit provides reagents to perform the methods described herein, such as PTA. In some instances, a kit further comprises reagents configured for gene editing (e.g., Crispr/cas9 or other method described herein). In some instances, a kit comprises a variant polymerase described herein.

In a related aspect, the invention provides a kit comprising a reverse transcriptase, a nucleic acid polymerase, one or more amplification primers, a mixture of nucleotides comprising one or more terminator nucleotides, and optionally instructions for use. In one embodiment of the kits of the invention, the nucleic acid polymerase is a strand displacing DNA polymerase. In one embodiment of the kits of the invention, the nucleic acid polymerase is selected from bacteriophage phi29 (Φ29) polymerase, genetically modified phi29 (Φ29) DNA polymerase, Klenow Fragment of DNA polymerase I, phage M2 DNA polymerase, phage phiPRD1 DNA polymerase, Bst DNA polymerase, Bst large fragment DNA polymerase, exo(−) Bst polymerase, exo(−)Bca DNA polymerase, Bsu DNA polymerase, VentR DNA polymerase, VentR (exo−) DNA polymerase, Deep Vent DNA polymerase, Deep Vent (exo−) DNA polymerase, IsoPol DNA polymerase, DNA polymerase I, Therminator DNA polymerase, T5 DNA polymerase, Sequenase, T7 DNA polymerase, T7-Sequenase, and T4 DNA polymerase. In one embodiment of the kits of the invention, the nucleic acid polymerase has 3′->5′ exonuclease activity and the terminator nucleotides inhibit such 3′->5′ exonuclease activity (e.g., nucleotides with modification to the alpha group [e.g., alpha-thio dideoxynucleotides], C3 spacer nucleotides, locked nucleic acids (LNA), inverted nucleic acids, 2′ fluoro nucleotides, 3′ phosphorylated nucleotides, 2′-O-Methyl modified nucleotides, trans nucleic acids). In one embodiment of the kits of the invention, the nucleic acid polymerase does not have 3′->5′ exonuclease activity (e.g., Bst DNA polymerase, exo(−) Bst polymerase, exo(−) Bca DNA polymerase, Bsu DNA polymerase, VentR (exo−) DNA polymerase, Deep Vent (exo−) DNA polymerase, Klenow Fragment (exo−) DNA polymerase, Therminator DNA polymerase). In one specific embodiment, the terminator nucleotides comprise modifications of the r group of the 3′ carbon of the deoxyribose. In one specific embodiment, the terminator nucleotides are selected from 3′ blocked reversible terminator comprising nucleotides, 3′ unblocked reversible terminator comprising nucleotides, terminators comprising 2′ modifications of deoxynucleotides, terminators comprising modifications to the nitrogenous base of deoxynucleotides, and combinations thereof. In one specific embodiment, the terminator nucleotides are selected from dideoxynucleotides, inverted dideoxynucleotides, 3′ biotinylated nucleotides, 3′ amino nucleotides, 3′-phosphorylated nucleotides, 3′-O-methyl nucleotides, 3′ carbon spacer nucleotides including 3′ C3 spacer nucleotides, 3′ C18 nucleotides, 3′ Hexanediol spacer nucleotides, acyclonucleotides, and combinations thereof.

### EXAMPLES

The following examples are set forth to illustrate more clearly the principle and practice of embodiments disclosed herein to those skilled in the art and are not to be construed as limiting the scope of any claimed embodiments. Unless otherwise stated, all parts and percentages are on a weight basis.

### Example 1

### Primary Template-Directed Amplification (PTA)

While PTA can be used for any nucleic acid amplification, it is particularly useful for whole genome amplification as it allows to capture a larger percentage of a cell genome in a more uniform and reproducible manner and with lower error rates than the currently used methods such as, e.g., Multiple Displacement Amplification (MDA), avoiding such drawbacks of the currently used methods as exponential amplification at locations where the polymerase first extends the random primers which results in random overrepresentation of loci and alleles and mutation propagation (see FIGS. 1A-1C).

Cell Culture

Human NA12878 (Coriell Institute) cells were maintained in RPMI media, supplemented with 15% FBS and 2 mM L-glutamine, and 100 units/mL of penicillin, 100 μg/mL of streptomycin, and 0.25 μg/mL of Amphotericin B (Gibco, Life Technologies). The cells were seeded at a density of 3.5×10⁵ cells/ml. The cultures were split every 3 days and were maintained in a humidified incubator at 37 C with 5% CO₂.

Single-Cell Isolation and WGA

After culturing NA12878 cells for a minimum of three days after seeding at a density of 3.5×10⁵ cells/ml, 3 mL of cell suspension were pelleted at 300×g for 10 minutes. The medium was then discarded and the cells were washed three times with 1 mL of cell wash buffer (1× PBS containing 2% FBS without Mg²⁺ or Ca²⁺) being spun at 300×g, 200×g and finally 100×g for 5 minutes. The cells were then resuspended in 500 μL of cell wash buffer. This was followed by staining with 100 nM of Calcein AM (Molecular Probes) and 100 ng/ml of propidium iodide (PI; Sigma-Aldrich) to distinguish the live cell population. The cells were loaded on a BD FACScan flow cytometer (FACSAria II) (BD Biosciences) that had been thoroughly cleaned with ELIMINase (Decon Labs) and calibrated using Accudrop fluorescent beads (BD Biosciences) for cell sorting. A single cell from the Calcein AM-positive, PI-negative fraction was sorted in each well of a 96 well plate containing 3 μL of PBS with 0.2% Tween 20 in the cells that would undergo PTA (Sigma-Aldrich). Multiple wells were intentionally left empty to be used as no template controls (NTC). Immediately after sorting, the plates were briefly centrifuged and placed on ice. Cells were then frozen at a minimum of overnight at −20° C. On a subsequent day, WGA Reactions were assembled on a pre-PCR workstation that provides a constant positive pressure of HEPA filtered air and which was decontaminated with UV light for 30 minutes before each experiment.

MDA was carried out using with modifications that have previously been shown to improve the amplification uniformity. Specifically, exonuclease-resistant random primers (ThermoFisher) were added to a lysis buffer/mix to a final concentration of 125 μM. 4 μL of the resulting lysis/denaturing mix was added to the tubes containing the single cells, vortexed, briefly spun and incubated on ice for 10 minutes. The cell lysates were neutralized by adding 3 μL of a quenching buffer, mixed by vortexing, centrifuged briefly, and placed at room temperature. This was followed by addition of 40 μl of amplification mix before incubation at 30° C. for 8 hours after which the amplification was terminated by heating to 65° C. for 3 minutes.

PTA was carried out by first further lysing the cells after freeze thawing by adding 2 μl a prechilled solution of a 1:1 mixture of 5% Triton X-100 (Sigma-Aldrich) and 20 mg/ml Proteinase K (Promega). The cells were then vortexed and briefly centrifuged before placing at 40 degrees for 10 minutes. 4 μl of lysis buffer/mix and 1 μl of 500 μM exonuclease-resistant random primer were then added to the lysed cells to denature the DNA prior to vortexing, spinning, and placing at 65 degrees for 15 minutes. 4 μl of room temperature quenching buffer was then added and the samples were vortexed and spun down. 56 μl of amplification mix (primers, dNTPs, polymerase, buffer) that contained alpha-thio-ddNTPs at equal ratios at a concentration of 1200 μM in the final amplification reaction. The samples were then placed at 30° C. for 8 hours after which the amplification was terminated by heating to 65° C. for 3 minutes.

After the amplification step, the DNA from both MDA and PTA reactions were purified using AMPure XP magnetic beads (Beckman Coulter) at a 2:1 ratio of beads to sample and the yield was measured using the Qubit dsDNA HS Assay Kit with a Qubit 3.0 fluorometer according to the manufacturer's instructions (Life Technologies).

Library Preparation

The MDA reactions resulted in the production of 40 μg of amplified DNA. 1 μg of product was enzymatically fragmented for 30 minutes following standard procedures. The samples then underwent standard library preparation with 15 μM of dual index adapters (end repair by a T4 polymerase, T4 polynucleotide kinase, and Taq polymerase for A-tailing) and 4 cycles of PCR. Each PTA reaction generated between 40-60 ng of material which was used for standard DNA sequencing library preparation. 2.5 μM adapters with UMIs and dual indices were used in the ligation with T4 ligase, and 15 cycles of PCR (hot start polymerase) were used in the final amplification. The libraries were then cleaned up using a double sided SPRI using ratios of 0.65× and 0.55× for the right and left sided selection, respectively. The final libraries were quantified using the Qubit dsDNA BR Assay Kit and 2100 Bioanalyzer (Agilent Technologies) before sequencing on the Illumina NextSeq platform. All Illumina sequencing platforms, including the NovaSeq, are also compatible with the protocol.

Data Analysis

Sequencing reads were demultiplexed based on cell barcode using Bcl2fastq. The reads were then trimmed using trimmomatic, which was followed by alignment to hg19 using BWA. Reads underwent duplicate marking by Picard, followed by local realignment and base recalibration using GATK 4.0. All files used to calculate quality metrics were downsampled to twenty million reads using Picard DownSampleSam. Quality metrics were acquired from the final bam file using qualimap, as well as Picard AlignmentSummaryMetrics and CollectWgsMetrics. Total genome coverage was also estimated using Preseq.

Variant Calling

Single nucleotide variants and Indels were called using the GATK UnifiedGenotyper from GATK 4.0. Standard filtering criteria using the GATK best practices were used for all steps in the process (https://software.broadinstitute.org/gatk/best-practices/). Copy number variants were called using Control-FREEC (Boeva et al., Bioinformatics, 2012, 28(3):423-5). Structural variants were also detected using CREST (Wang et al., Nat Methods, 2011, 8(8):652-4).

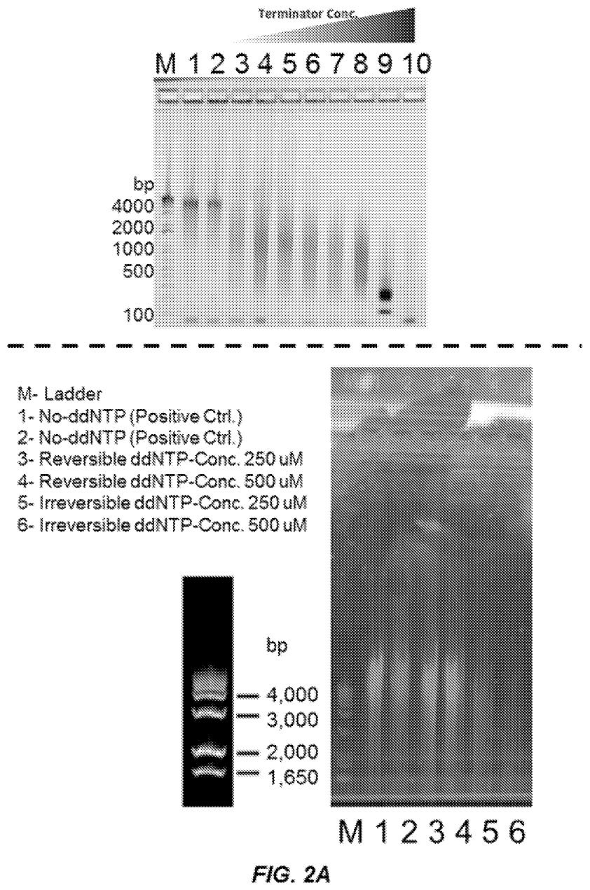

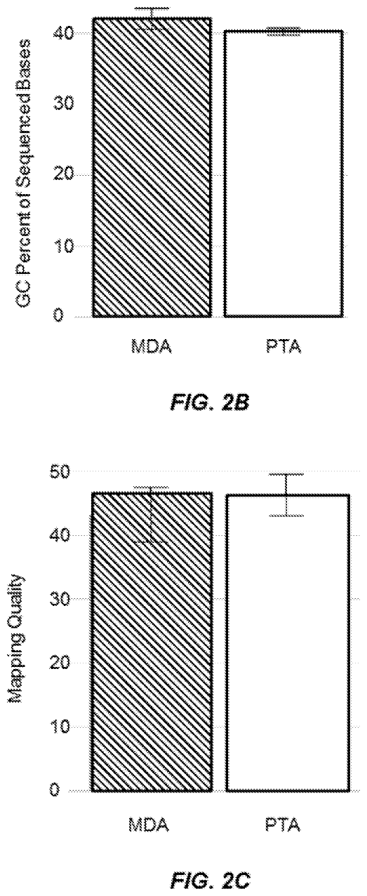

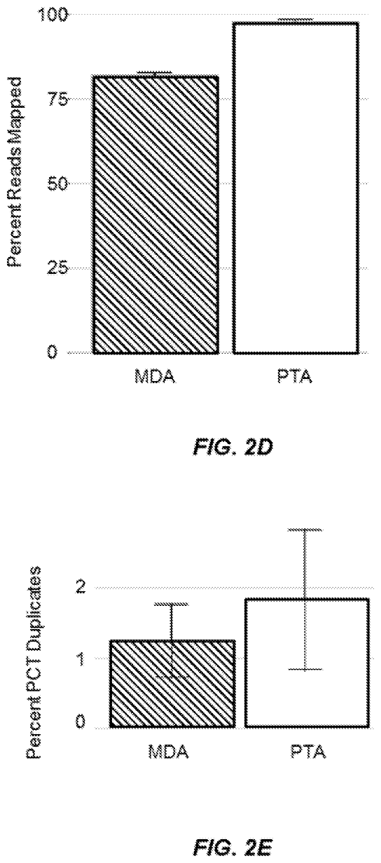

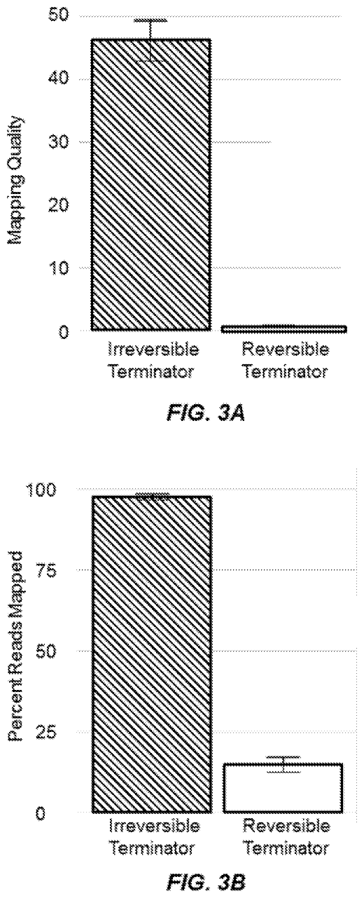

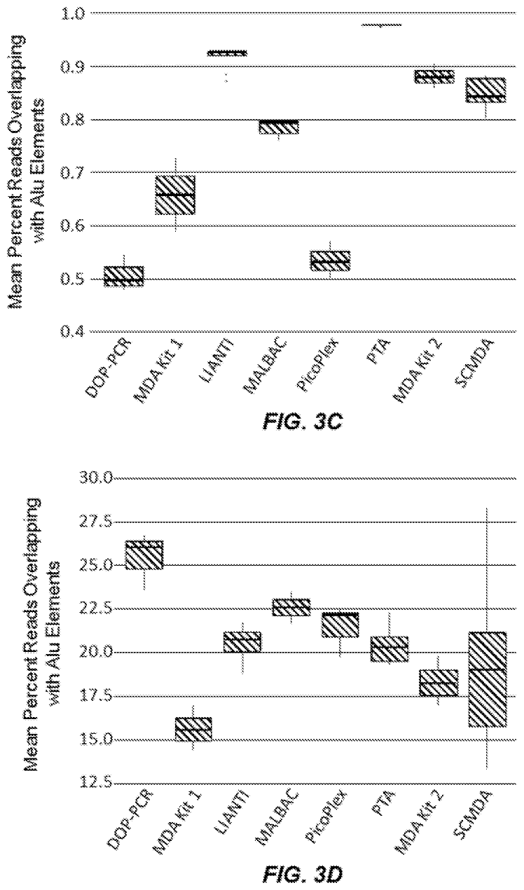

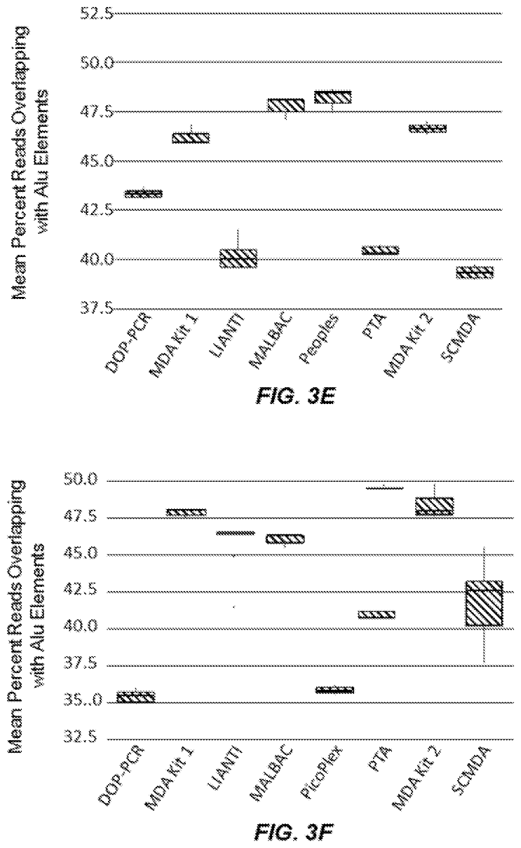

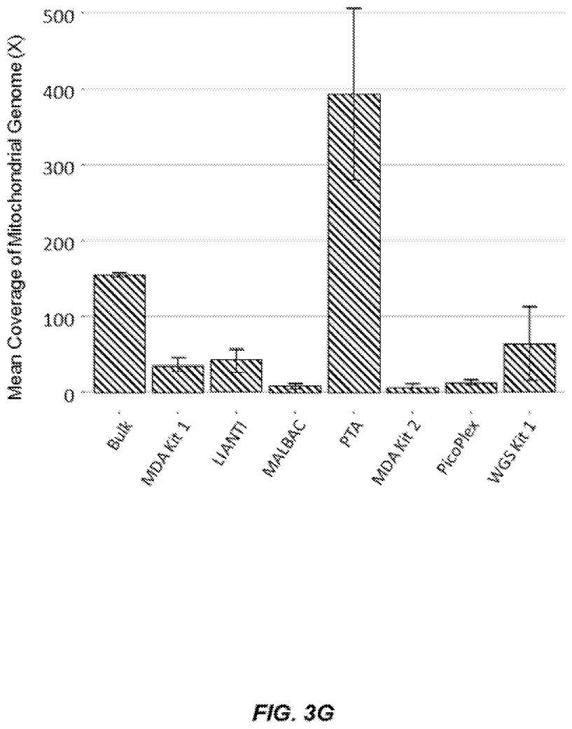

Results
As shown in FIG. 3A and FIG. 3B, the mapping rates and mapping quality scores of the amplification with dideoxynucleotides (“reversible”) alone are 15.0+/−2.2 and 0.8+/−0.08, respectively, while the incorporation of exonuclease-resistant alpha-thio dideoxynucleotide terminators (“irreversible”) results in mapping rates and quality scores of 97.9+/−0.62 and 46.3+/−3.18, respectively. Experiments were also run using a reversible ddNTP, and different concentrations of terminators. (FIG. 2A, bottom)

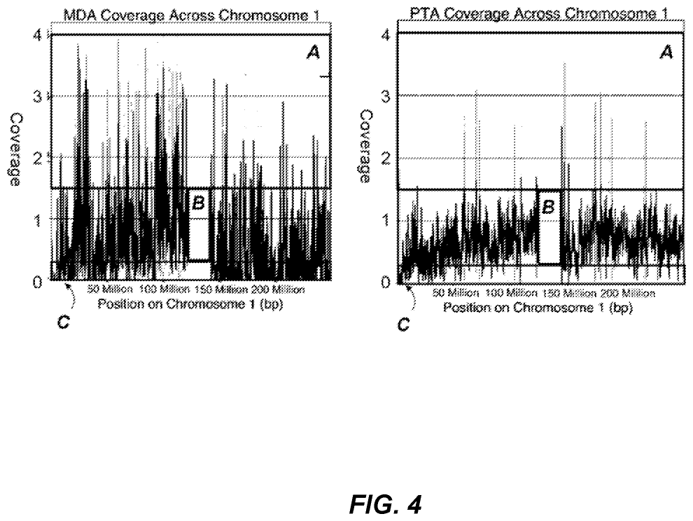

FIGS. 2B-2E show the comparative data produced from NA12878 human single cells that underwent MDA (following the method of Dong, X. et al., Nat Methods. 2017, 14(5):491-493) or PTA. While both protocols produced comparable low PCR duplication rates (MDA 1.26%+/−0.52 vs PTA 1.84%+/−0.99). and GC% (MDA 42.0+/−1.47 vs PTA 40.33+/−0.45), PTA produced smaller amplicon sizes. The percent of reads that mapped and mapping quality scores were also significantly higher for PTA as compared to MDA (PTA 97.9+/−0.62 vs MDA 82.13+/−0.62 and PTA 46.3+/−3.18 vs MDA 43.2+/−4.21, respectively). Overall, PTA produces more usable, mapped data when compared to MDA. FIG. 4 shows that, as compared to MDA, PTA has significantly improved uniformity of amplification with greater coverage breadth and fewer regions where coverage falls to near 0. The use of PTA allows identifying low frequency sequence variants in a population of nucleic acids, including variants which constitute ≥0.01% of the total sequences. PTA can be successfully used for single cell genome amplification.

### Example 2

### Massively Parallel Single-Cell DNA Sequencing

Using PTA, a protocol for massively parallel DNA sequencing is established. First, a cell barcode is added to the random primer. Two strategies to minimize any bias in the amplification introduced by the cell barcode is employed: 1) lengthening the size of the random primer and/or 2) creating a primer that loops back on itself to prevent the cell barcode from binding the template (FIG. 6B). Once the optimal primer strategy is established, up to 384 sorted cells are scaled by using, e.g., Mosquito HTS liquid handler, which can pipette even viscous liquids down to a volume of 25 nL with high accuracy. This liquid handler also reduces reagent costs approximately 50-fold by using a 1 μL PTA reaction instead of the standard 50 μL reaction volume.

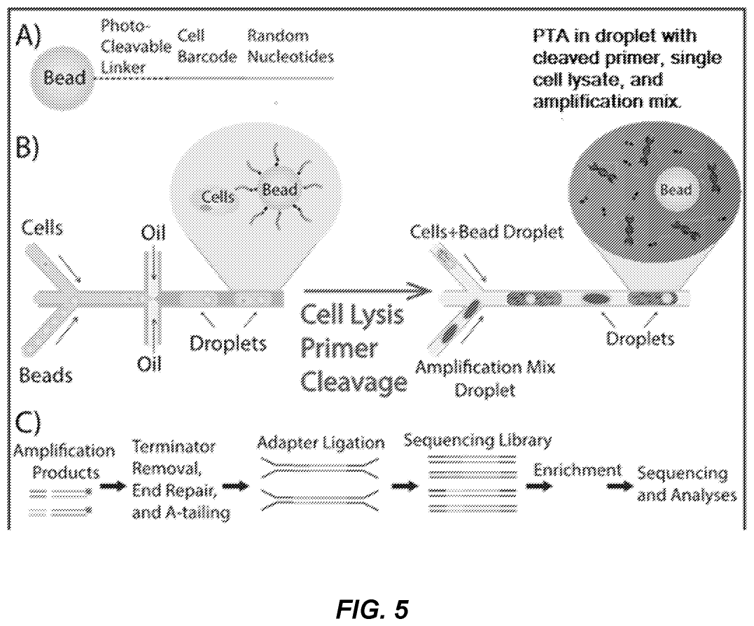

The amplification protocol is transitioned into droplets by delivering a primer with a cell barcode to a droplet. Solid supports, such as beads that have been created using the split-and-pool strategy, are optionally used. Suitable beads are available e.g., from ChemGenes. The oligonucleotide in some instances contains a random primer, cell barcode, unique molecular identifier, and cleavable sequence or spacer to release the oligonucleotide after the bead and cell are encapsulated in the same droplet. During this process, the template, primer, dNTP, alpha-thio-ddNTP, and polymerase concentrations for the low nanoliter volume in the droplets are optimized. Optimization in some instances includes use of larger droplets to increase the reaction volume. As seen in FIG. 5, this process requires two sequential reactions to lyse the cells, followed by WGA. The first droplet, which contains the lysed cell and bead, is combined with a second droplet with the amplification mix. Alternatively or in combination, the cell is encapsulated in a hydrogel bead before lysis and then both beads may be added to an oil droplet. See Lan, F. et al., Nature Biotechnol., 2017, 35:640-646).

Additional methods include use of microwells, which in some instances capture 140,000 single cells in 20-picoliter reaction chambers on a device that is the size of a 3″×2″ microscope slide. Similarly to the droplet-based methods, these wells combine a cell with a bead that contains a cell barcode, allowing massively parallel processing. See Gole et al., Nature Biotechnol., 2013, 31:1126-1132).

### Example 3

### Phi29 Variant Polymerases

Following the general methods of Example 1, the PTA method is conducted with a variant polymerase having any one of SEQ ID NOs: 11-15. Variant polymerases are expressed from plasmids or genomic integration in a suitable host, purified, and used with the PTA method. Sequencing metrics such as uniformity and base calling are evaluated and compared to a control experiment using Phi29 polymerase of SEQ ID NO: 1.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of nucleic acid amplification comprising:
a. providing a sample comprising at least one target nucleic acid molecule;
b. contacting the sample with at least one amplification primer, at least one polymerase, and a mixture of nucleotides, wherein the mixture of nucleotides comprises at least one terminator nucleotide which terminates nucleic acid replication by the polymerase, wherein the polymerase comprises at least three mutations relative to SEQ ID NO:1, wherein at least two mutations are at positions 370-395 relative to SEQ ID NO: 1, and wherein the polymerase has increased processivity, increased strand displacement activity, increased template or primer binding, decreased error rate, increased 3′->5′ exonuclease activity, increased nucleotide selectivity, or increased temperature stability relative to a polymerase comprising SEQ ID NO: 1; and
c. amplifying the at least one target nucleic acid molecule to generate a plurality of terminated amplification products.

2. The method of claim 1, wherein increased nucleotide selectivity comprises increased affinity for non-canonical nucleotides.

3. The method of claim 2, wherein the non-canonical nucleotides comprise dideoxynucleotides.

4. The method of claim 1, further comprising ligating the molecules obtained in step (c) to adaptors, thereby generating a library of amplification products.

5. The method of claim 4, wherein the method further comprises sequencing the library of amplification products.

6. The method of claim 5, wherein the method further comprises comparing the sequences of amplification products to at least one reference sequence to identify at least one mutation.

7. The method of claim 1, wherein the sample comprises genomic DNA.

8. The method of claim 1, wherein the sample is a single cell.

9. The method of claim 8, wherein the single cell is a mammalian cell.

10. The method of claim 8, wherein the single cell is a human cell.

11. The method of any one of claims 1-10, wherein at least some of the amplification products comprise a barcode.

12. The method of any one of claims 1-10, wherein at least some of the amplification products comprise at least two barcodes.

13. The method of claim 11 or 12, wherein the barcode comprises a cell barcode.

14. The method of claim 11 or 12, wherein the barcode comprises a sample barcode.

15. The method of any one of claims 1-14, wherein at least some of the amplification primers comprise a unique molecular identifier (UMI).

16. The method of any one of claims 1-14, wherein at least some of the amplification primers comprise at least two unique molecular identifiers (UMIs).

17. The method of any one of claims 1-16, wherein the method further comprises an additional amplification step using PCR.

18. The method of any one of claims 1-17, wherein the method further comprises removing at least one terminator nucleotide from the terminated amplification products prior to ligation to adapters.

19. The method of claim 8, wherein single cells are isolated from the population using a method comprising a microfluidic device.

20. The method of claim 6, wherein the at least one mutation occurs in no more than 1% of the amplification product sequences.

21. The method of claim 6, wherein the at least one mutation occurs in no more than 0.1% of the amplification product sequences.

22. The method of claim 6, wherein the at least one mutation occurs in no more than 0.01% of the amplification product sequences.

23. The method of claim 6, wherein the at least one mutation occurs in no more than 0.001% of the amplification product sequences.

24. The method of claim 6, wherein the at least one mutation occurs in no more than 0.0001% of the amplification product sequences.

25. The method of claim 6, wherein the at least one mutation is present in a region of a sequence correlated with a genetic disease or condition.

26. A variant polymerase comprising SEQ ID NO: 1, wherein the polymerase comprises at least two mutations at positions 370-395 relative to SEQ ID NO: 1, and wherein the polymerase has increased processivity, increased strand displacement activity, increased template or primer binding, decreased error rate, increased 3′->5′ exonuclease activity, increased nucleotide selectivity, or increased temperature stability relative to a polymerase comprising SEQ ID NO: 1.

27. The polymerase of claim 26, wherein the polymerase comprises at least three mutations at positions 370-395 relative to SEQ ID NO: 1.

28. The polymerase of claim 26, wherein the polymerase comprises at least four mutations at positions 370-395 relative to SEQ ID NO: 1.

29. The polymerase of claim 26, wherein at least one mutation is at positions 1-369 or 396-575 relative to SEQ ID NO: 1.

30. The polymerase of claim 26, wherein the at least one mutation comprises a substitution, deletion, or addition.

31. The polymerase of claim 26, wherein the at least one mutation is at positions A382, L386, M385, or E375.

32. The polymerase of claim 30 or 31, wherein the at least one mutation comprises at least one substitution.

33. The polymerase of claim 32, wherein the at least one substitution is at an alanine, glycine, leucine, methionine, glutamic acid, or cysteine position of SEQ ID NO: 1.

34. The polymerase of claim 33, wherein the at least one substitution is from alanine, glycine, leucine, methionine, glutamic acid, or cysteine to phenylalanine, tyrosine, or tryptophan.

35. The polymerase of claim 26, wherein the polymerase comprises a mutation at P300.

36. The polymerase of claim 35, wherein the polymerase comprises a substitution at P300.

37. The polymerase of claim 36, wherein the polymerase comprises a substitution at P300 to leucine, isoleucine, alanine, glycine, methionine, or cysteine.

38. The polymerase of claim 26, wherein the polymerase comprises a mutation at K512.

39. The polymerase of claim 38, wherein the polymerase comprises a substitution at K512.

40. The polymerase of claim 39, wherein the polymerase comprises a substitution at K512 to alanine, aspartic acid, glutamic acid, tryptophan, tyrosine, phenylalanine, leucine, or histidine.

41. The polymerase of claim 26, wherein the polymerase comprises at least one mutation at M8, V51, M97, L123, G197, K209, E221, E239, Q497, K512, E515, or F526.

42. The polymerase of claim 41, wherein the at least one mutation at M8, V51, M97, L123, G197, K209, E221, E239, Q497, K512, E515, or F526 is at least one substitution.

43. The polymerase of claim 42, wherein the at least one substitution is M8R, V51A, M97T, L123S, G197D, K209E, E221K, E239G, Q497P, K512E, E515A, or F526L.

44. The polymerase of claim 26, wherein the polymerase comprises at least one mutation at M8, D12, N62, M97, M102, H116, K135, H149, K157, M188, 1242, S252, Y254, G320, L328, 1370, K371, T372, K373, S374, E375, T368, Y369, T372, T373, 1378, K379, N387, Y390, Y405, E408, G413, D423, 1442, Y449, D456, K478, L480, V509, D510, K512, V514, E515, M554.

45. The polymerase of claim 44, wherein the at least one mutation is at least one substitution.

46. The polymerase of claim 44, wherein the at least one substitution is D12A/E375W/T372D; D12A/E375W/T372E; D12A/E375W/T372R/K478D; D12A/E375W/T372R/K478E; D12A/E375W/T372K/K478D; D12A/E375W/T372K/D478E; D12A/E375W/K135D; D12A/E375W/K135E; D12A/E375W/K512D; D12A/E375W/K512E; D12A/E375W/E408K; D12A/E375W/E408R; D12A/E375W/T368D/L480K; D12A/E375W/T368E/L480K; D12A/D456N; N62D/D456N; D12A/D456A; N62D/D456A; D12A/D456S; N62D/D456S; N62D/E375M; N62D/E375L; N62D/E3751; N62D/E375F; N62D/E375D; D12A/K512W; N62D/K512W; D12A/K512Y; N62D/K512Y; D12A/K512F; N62D/K512F; D12A/E375W/K512L; N62D/E375W/K512L; D12A/E375W/K512Y; N52D/E375W/K512Y; D12A/E375W/K512F; N62D/E375W/K512F; D12A/E375Y/K512L; N62D/E375Y/K512L; D12A/E375Y/K512Y; N62D/E375Y/K512Y; D12A/E375Y/K512F; N62D/E375Y/K512F; D12A/E375W/K512H; N62D/E375W/K512H; D12A/E375Y/K512H; N62D/E375Y/K512H; D12A/D510F; N62D/D510F; D12A/D510Y; N62D/D510Y; D12A/D510W; N62D/D510W; D12A/E375W/D510F; N62D/E375W/D510F; D12A/E375W/D510Y; N62D/E375W/D510Y; D12A/E375W/D510W; N62D/E375W/D510W; D12A/E375W/D510W/K512L; N62D/E375W/D510W/K512L; D12A/E375W/D510W/K512F; N62D/E375W/D510W/K512F; D12A/E375W/D510H; N62D/E375W/D510H; D12A/E375W/D510H/K512H; N62D/E375W/D510H/K512H; D12A/E375W/D510H/K512F; N62D/E375W/D510H/K512F; D12A/V509Y; N62D/V509Y; D12A/V509W; N62D/V509W; D12A/V509F; N62D/V509F; D12A/V514Y; N62D/V514Y; D12A/V514W; N62D/V514W; D12A/V514F; N62D/V514F; D12S; D12N; D12Q; D12K; D12A/N62D/Y254F; N62D/Y254V; N62D/Y254A; N62D/Y390F; N62D/Y390A; N62D/S252A; N62D/N387A; N62D/K157E; N62D/I242H; N62D/Y259S; N62D/G320C; N62D/L328V; N62D/T368M; N62D/T368G; N62D/Y369R; N62D/Y369H; N62D/Y369E; N62D/I370V; N62D/I370K; N62D/K371Q; N62D/T372N; N62D/T372D; N62D/T372R; N62D/T372L; N62D/T373A; N62D/T373H; N62D/S374E; N62D/I378K; N62D/K379E; N62D/K379T; N62D/N387D; N62D/Y405V; N62D/L408D; N62D/G413D; N62D/D423V; N62D/I442V; N62D/Y449F; N62D/D456V; N62D/L480M; N62D/V509K; N62D/V509I; N62D/D510A; N62D/V514I; N62D/V514K; N62D/E515K; N62D/D523T; N62D/H149Y/E375W/M554S; M8S/N62D/M102S/H116Y/M188S/E375W; N62D/M97S/E375W; M8S/N62D/M97S/M102S/M188S/E375W/M554S; or M8AN62D/M97A/M102A/M188A/E375W/M554A.

47. A variant polymerase, wherein the polymerase comprises a sequence having at least 70% identity to any one of SEQ ID NOS: 4-15.

48. The polymerase of claim 47, wherein the polymerase comprises a sequence having at least 80% identity to any one of SEQ ID NOS: 4-15.

49. The polymerase of claim 47, wherein the polymerase comprises a sequence having at least 90% identity to any one of SEQ ID NOS: 4-15.

50. The polymerase of claim 47, wherein the polymerase comprises a sequence having at least 95% identity to any one of SEQ ID NOS: 4-15.

51. The polymerase of claim 47, wherein the polymerase comprises a sequence having at least 97% identity to any one of SEQ ID NOS: 4-15.

52. A variant polymerase, wherein the polymerase comprises a sequence of any one of SEQ ID NOS: 4-10.

53. A variant polymerase, wherein the polymerase comprises a sequence of any one of SEQ ID NOS: 11-15.

54. A variant polymerase comprising a polypeptide having the structure of Formula I:
X¹X²X³X⁴X⁵X⁶X⁷X⁸X⁹X¹⁰X¹¹X¹²X¹³X¹⁴X¹⁵X¹⁶X¹⁷X¹⁸X¹⁹X²⁰X²¹X²²X²³X²⁴X²⁵X²⁶ Formula (I);
wherein
X¹, X⁷, X⁸, X⁹, X¹², X¹³, X¹⁵, X¹⁶, X¹⁷, X²⁰, X²¹, X²², X²⁴, and X²⁵ are each independently an aromatic or non-polar amino acid;
X³, X⁴, X⁵, X¹¹, X¹⁸, X¹⁹, and X²⁶ are each independently polar amino acids;
X², X¹⁰, X¹⁴, and X²³ are each independently positively charged amino acids; and
X⁶ is an aromatic or negatively charged amino acid, and wherein the polymerase comprises increased processivity, increased strand displacement activity, increased template or primer binding, decreased error rate, increased 3′->5′ exonuclease activity, increased nucleotide selectivity, or increased temperature stability relative to a polymerase comprising SEQ ID NO: 1.

55. The polymerase of claim 54, wherein X²¹ and X²⁴ are each independently a non-polar aromatic amino acid.

56. The polymerase of claim 54, wherein at least one of X¹, X⁷, X⁸, X⁹, X¹², X¹³, X¹⁵, X¹⁶, X¹⁷, X²⁰, X²¹, X²⁵ are each independently an aromatic amino acid.

57. The polymerase claim 54, wherein at least one of X¹, X⁷, X⁸, X⁹, X¹², X¹³, X¹⁵, X¹⁶, X¹⁷, X²⁰, X²¹, X²⁵ are each independently tyrosine, phenylalanine, or tryptophan.

58. The polymerase of claim 54, wherein at least one of X¹, X⁷, X⁸, X⁹, X¹², and X¹³ are each independently tyrosine, phenylalanine, or tryptophan.

59. The polymerase of claim 54, wherein at least one of X¹⁵, X^{16,} X¹⁷, X²⁰, X²¹, X²⁵ are each independently tyrosine, phenylalanine, or tryptophan.

60. The polymerase of claim 54, wherein at least two of X¹, X⁷, X⁸, X⁹, X¹², X¹³, X¹⁵, X¹⁶, X¹⁷, X²⁰, X²¹, X²⁵ are each independently tyrosine, phenylalanine, or tryptophan.

61. The polymerase of claim 54, wherein at least one of X¹, X⁶, X⁷, X⁸, X⁹, X¹², X¹³, X¹⁵, X¹⁶, X¹⁷, X²⁰, X²¹, X²⁵ are each independently tyrosine, phenylalanine, or tryptophan.

62. The polymerase of claim 54, wherein at least one of X¹, X⁷, X⁸, X⁹, X¹², X¹³, X¹⁵, X¹⁶, X¹⁷, X²⁰, X²¹, X²⁵ are each independently valine or isoleucine.

63. The polymerase of claim 54 or 55, wherein X¹⁶ is an aromatic amino acid.

64. The polymerase of claim 63, wherein X¹⁶ is tyrosine, phenylalanine, or tryptophan.

65. The polymerase of any one of claim 54, 55, or 63, wherein X¹⁷ is glycine or alanine.

66. The polymerase of any one of claim 54, 55, 63, or 65, wherein X⁶ is an aromatic amino acid.

67. The polymerase of any one of claims 66, wherein X⁶ is tyrosine, phenylalanine, or tryptophan.

68. A kit for nucleic acid sequencing comprising:
a. at least one amplification primer;
b. at least one nucleic acid polymerase of any one of claims 26-67;
c. a mixture of at least two nucleotides, wherein the mixture of nucleotides comprises at least one terminator nucleotide which terminates nucleic acid replication by the polymerase; and
d. instructions for use of the kit to perform nucleic acid sequencing.

69. The kit of claim 68, wherein the at least one amplification primer is a random primer.

70. The kit of claim 68, wherein the nucleic acid polymerase is a DNA polymerase.

71. The kit of claim 70, wherein the DNA polymerase is a strand displacing DNA polymerase.

72. The kit of any one of claims 68-71, wherein the least one terminator nucleotide comprises modifications of the r group of the 3′ carbon of the deoxyribose.

73. The kit of any one of claims 68-72, wherein the at least one terminator nucleotide is selected from the group consisting of 3′ blocked reversible terminator containing nucleotides, 3′ unblocked reversible terminator containing nucleotides, terminators containing 2′ modifications of deoxynucleotides, terminators containing modifications to the nitrogenous base of deoxynucleotides, and combinations thereof.

74. The kit of any one of claims 68-73, wherein the at least one terminator nucleotide is selected from the group consisting of dideoxynucleotides, inverted dideoxynucleotides, 3′ biotinylated nucleotides, 3′ amino nucleotides, 3′-phosphorylated nucleotides, 3′ methyl nucleotides, 3′ carbon spacer nucleotides including 3′ C3 spacer nucleotides, 3′ C18 nucleotides, 3′ Hexanediol spacer nucleotides, acyclonucleotides, and combinations thereof.

75. The kit of any one of claims 68-74, wherein the at least one terminator nucleotide are selected from the group consisting of nucleotides with modification to the alpha group, C3 spacer nucleotides, locked nucleic acids (LNA), inverted nucleic acids, 2′ fluoro nucleotides, 3′ phosphorylated nucleotides, 2′-O-Methyl modified nucleotides, and trans nucleic acids.

76. The kit of any one of claims 68-75, wherein the nucleotides with modification to the alpha group are alpha-thio dideoxynucleotides.

77. The kit of any one of claims 68-76, wherein the amplification primers are 4 to 70 nucleotides in length.

78. The kit of any one of claims 68-77, wherein the at least one amplification primer is 4 to 20 nucleotides in length.

79. The kit of any one of claims 68-78, wherein the at least one amplification primer comprises a randomized region.

80. The kit of claim 79, wherein the randomized region is 4 to 20 nucleotides in length.

81. The kit of claim 79 or 80, wherein the randomized region is 8 to 15 nucleotides in length.

82. The kit of any one of claims 68-81, wherein the kit further comprises a library preparation kit.

83. The kit of claim 82, wherein the library preparation kit comprises one or more of:
a. at least one polynucleotide adapter;
b. at least one high-fidelity polymerase;
c. at least one ligase;
d. a reagent for nucleic acid shearing; and
e. at least one primer, wherein the primer is configured to bind to the adapter.

84. The kit of any one of claims 68-83, wherein the kit further comprises reagents configured for gene editing.


SEQUENCE LISTING (15 sequences)

SEQ ID NO: 1 - protein, 575 aa, Bacillus virus phi29 [sequence omitted]

SEQ ID NO: 2 - protein, 32 aa, Artificial Sequence
  Description of Artificial Sequence Synthetic polypeptide
TQDPIEKILLDYRQKAIKLLANSFYGYYGYAK

SEQ ID NO: 3 - protein, 26 aa, Artificial Sequence
  Description of Artificial Sequence Synthetic peptide
IKTTSEGAIKQLAKLMLNSLYGKFAS

SEQ ID NO: 4 - protein, 26 aa, Artificial Sequence
  Description of Artificial Sequence Synthetic peptide
IKTTSEGAIKQLYKLMLNSLYGKFAS

SEQ ID NO: 5 - protein, 26 aa, Artificial Sequence
  Description of Artificial Sequence Synthetic peptide
IKTTSEGAIKQLWKLMLNSLYGKFAS

SEQ ID NO: 6 - protein, 26 aa, Artificial Sequence
  Description of Artificial Sequence Synthetic peptide
IKTTSEGAIKQLAKLMYNSLYGKFAS

SEQ ID NO: 7 - protein, 26 aa, Artificial Sequence
  Description of Artificial Sequence Synthetic peptide
IKTTSEGAIKQLAKLMWNSLYGKFAS

SEQ ID NO: 8 - protein, 26 aa, Artificial Sequence
  Description of Artificial Sequence Synthetic peptide
IKTTSEGAIKQLAKLWANSLYGKFAS

SEQ ID NO: 9 - protein, 26 aa, Artificial Sequence
  Description of Artificial Sequence Synthetic peptide
IKTTSEGAIKQLAKLMLYSLYGKFAS

SEQ ID NO: 10 - protein, 26 aa, Artificial Sequence
  Description of Artificial Sequence Synthetic peptide
IKTTSEGAIKQLAKLMLWSLYGKFAS

SEQ ID NO: 11 - protein, 575 aa, Artificial Sequence
  Description of Artificial Sequence Synthetic polypeptide
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLWLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 12 - protein, 575 aa, Artificial Sequence
  Description of Artificial Sequence Synthetic polypeptide
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMANSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 13 - protein, 575 aa, Artificial Sequence; identical to SEQ ID NO: 11
  Description of Artificial Sequence Synthetic polypeptide

SEQ ID NO: 14 - protein, 575 aa, Artificial Sequence
  Description of Artificial Sequence Synthetic polypeptide
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSWGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK

SEQ ID NO: 15 - protein, 575 aa, Artificial Sequence
  Description of Artificial Sequence Synthetic polypeptide
MKHMPRKMYSCDFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYILTIQIKRSRFYKGNEYLKSSGGEIADLWLSNVDLELMKEHYDLYNVEYISGLKFKATTGLFKDFIDKWTYIKTTSWGAIKQLAKLMANSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIK